(19) United States
(12) Patent Application Publication
(10) Pub. No.: US 2025/0025581 A1
(43) Pub. Date: Jan. 23, 2025

(54) Medical Imaging of Glycogen Synthase Kinase-3 with a PET Probe

(71) Applicant: Mayo Foundation for Medical Education and Research, Rochester, MN (US)

(72) Inventors: Mukesh K. Pandey, Rochester, MN (US); Val J. Lowe, Rochester, MN (US); Aditya Bansal, Rochester, MN (US); Heather M. Stein, Rochester, MN (US); Surendra Reddy Gundam, Rochester, MN (US)

(21) Appl. No.: 18/711,840

(22) PCT Filed: Nov. 18, 2022
(86) PCT No.: PCT/US2022/050454

§ 371 Date: May 20, 2024

Related U.S. Application Data
(60) Provisional application No. 63/281,173, filed on Nov. 19, 2021.

(51) Int. Cl. A61K 51/04; C07B 59/00

(52) U.S. Cl. CPC A61K 51/0455; C07B 59/002; A61K 2121/00; A61K 2123/00; C07B 2200/05

(57) ABSTRACT

Disclosed are compounds of formula (1), formula (2), formula (75), formula (80) and formula (90): wherein R¹, R², R³, R⁴, and R⁵ can be selected from the group consisting of hydrogen, unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R¹ is replaced with a positron emitter. Also disclosed are methods for in vivo imaging of a subject using the compound of formula (1) or formula (2) or formula (75) or formula (80) or formula (90).

### CROSS-REFERENCES TO RELATED APPLICATIONS

This application is based on, claims benefit of, and claims priority to U.S. Application No. 63/281,173 filed on Nov. 19, 2021, which is hereby incorporated by reference herein in its entirety for all purposes.

### STATEMENT REGARDING FEDERALLY SPONSORED RESEARCH

Not Applicable.

### BACKGROUND OF THE INVENTION

### 1. Field of the Invention

This invention relates to selective and blood brain barrier permeable positron emission tomography (PET) imaging probes and their use in medical imaging.

### 2. Description of the Related Art

Glycogen synthase kinase-3 (GSK-3) is an enzyme with two isoforms, GSK-3α and GSK-3β. These isoforms transfer a phosphate group from adenosine triphosphate (ATP) to its target substrates. The process of transfer of phosphate group to its substrates is called phosphorylation. The mechanism of phosphorylation regulates various complex biological processes, including metabolism (e.g. glucose regulation), cell signaling, cellular transport, apoptosis, proliferation, and intracellular communication.

The role of GSK-3 in glucose metabolism has been extensively studied as it regulates the conversion of glucose to glycogen and found that elevated expression and over-activity of GSK-3 are associated with insulin resistance in type 2 diabetes. As a result, GSK-3 inhibitors are being developed for the treatment of type 2 diabetes. Furthermore, elevated GSK-3 levels in bipolar disorder have been documented in both preclinical and clinical studies. Specifically, higher levels of GSK-3α and GSK-3β were found in peripheral blood mononuclear cells of bipolar disorder subjects than in those of healthy controls. GSK-3β is also associated with several neurodegenerative diseases, including Alzheimer's disease (AD), Parkinson's disease, and Huntington's disease.

GSK-3 has also been implicated in dementia, atherosclerosis, congenital myotonic dystrophy, myotonic dystrophy, myelofibrosis, obesity, autism spectrum disorder, cancers including but not limited to osteosarcoma, neuroendocrine tumors, brain cancer, small cell lung carcinoma, prostate cancer, leukemia, pancreatic cancer, salivary gland carcinoma, sarcoma, lymphoma (Hodgkin's lymphoma or Non-Hodgkin's lymphoma), ovarian cancer, renal cancer, bone cancer, breast cancer, chronic lymphocytic leukemia, colorectal cancer, lung cancer, bladder cancer, glioblastoma, neuroblastoma, thyroid cancer. The cancers could be primary or malignant/metastatic, responding to treatment, refractory, or recurrent/relapsed.

GSK-3 has been found to be directly involved in phosphorylation of neuronal tau protein. The subsequent misfolding of tau to form fibrillary tangles is a central feature of the progression of AD symptoms. Noninvasive mapping of GSK-3 and its activity in normal and AD pathology would be instrumental to better understand the role of GSK-3 in the pathophysiology of AD. However, a major barrier to noninvasive study of GSK-3 in AD is the absence of a selective and blood brain barrier (BBB) permeable positron emission tomography (PET) imaging probe. Although various attempts have been made to synthesize BBB permeable and selective PET probes for GSK-3, these efforts have resulted in compounds that are not or minimally permeable to BBB or have significant off-target binding.

Therefore, there exists an unmet need to develop a noninvasive imaging probe to understand the role of GSK-3 in neurodegenerative diseases, including Alzheimer's disease, Parkinson's disease, and Huntington's disease.

### SUMMARY OF THE INVENTION

The present disclosure provides compounds of formula (1)

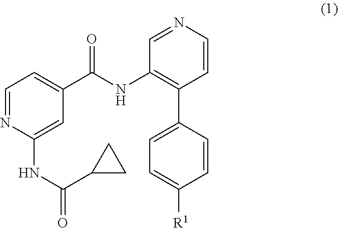

wherein R¹ is selected from the group consisting of unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, substituted alkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R¹ is replaced with a positron emitter. In one embodiment, the positron emitter is ¹⁸F, and the compound is a blood brain barrier permeable and selective positron emission tomography (PET) probe for GSK-3.

The present disclosure provides compounds of formula (2):

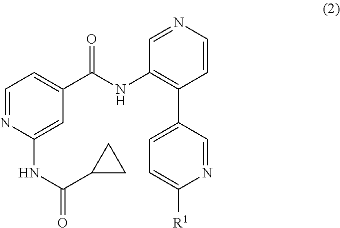

wherein R² is selected from the group consisting of unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R² is replaced with a positron emitter. In one embodiment, the positron emitter is ¹⁸F, and the compound is a blood brain barrier permeable and selective positron emission tomography (PET) probe for GSK-3.

The present disclosure provides compounds of formula (75):

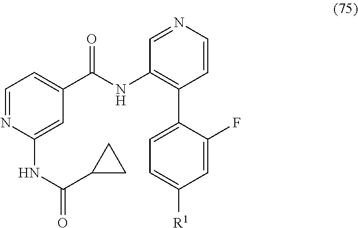

wherein R³ is selected from the group consisting of hydrogen, unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R³ is replaced with a positron emitter. In one embodiment, the positron emitter is ¹⁸F, and the compound is a blood brain barrier permeable and selective positron emission tomography (PET) probe for GSK-3.

The present disclosure provides compounds of formula (80):

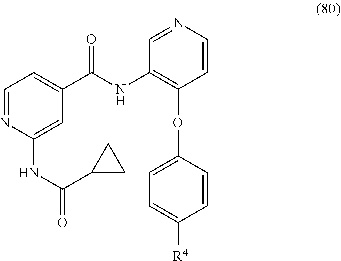

wherein R⁴ is selected from the group consisting of hydrogen, unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R⁴ is replaced with a positron emitter. In one embodiment, the positron emitter is ¹⁸F, and the compound is a blood brain barrier permeable and selective positron emission tomography (PET) probe for GSK-3.

The present disclosure provides compounds of formula (90):

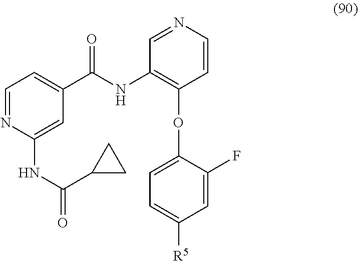

wherein R⁵ is selected from the group consisting of hydrogen, unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R⁵ is replaced with a positron emitter. In one embodiment, the positron emitter is ¹⁸F, and the compound is a blood brain barrier permeable and selective positron emission tomography (PET) probe for GSK-3.

In another aspect, the present disclosure provides a method for in vivo imaging of a subject, wherein the method comprises: (a) administering to the subject the compound of formula (1) or formula (2) or formula (75), or formula (80) or formula (90); (b) waiting a time sufficient to allow the compound to accumulate at a tissue or cell site to be imaged; and (c) imaging the cells or tissues with a non-invasive imaging technique. The non-invasive imaging technique can be selected from positron emission tomography imaging, positron emission tomography with computed tomography imaging, or positron emission tomography with magnetic resonance imaging.

In yet another aspect, the present disclosure provides a method for detecting GSK-3 in a subject, wherein the method comprises: (a) administering to the subject the compound of formula (1) or formula (2) or formula (75), or formula (80) or formula (90); (b) waiting a time sufficient to allow the compound to accumulate at a tissue or cell site to be imaged; and (c) imaging the cells or tissues with a non-invasive imaging technique. The non-invasive imaging technique can be selected from positron emission tomography imaging, positron emission tomography with computed tomography imaging, or positron emission tomography with magnetic resonance imaging.

In still another aspect, the present disclosure provides a method for the treatment of a condition involving GSK-3 activity, wherein the method comprises: administering to a subject having the condition a therapeutically effective amount of the compound of formula (1) or formula (2) or formula (75), or formula (80) or formula (90). The condition can be a cancer, a liver disease, a neurodegenerative disease, a psychiatric disease, or Alzheimer's disease.

In yet another aspect, the present disclosure provides a method for detecting or ruling out a condition involving GSK-3 activity in a subject, wherein the method comprises: (a) administering to a subject a detectable amount of the compound of formula (1) or formula (2) or formula (75), or formula (80) or formula (90) wherein the compound is targeted to GSK-3 at a tissue or cell site in the subject; and (b) acquiring an image of the cells or tissues to detect the presence or absence of GSK-3 in the subject. The image can be acquired using a non-invasive imaging technique selected from positron emission tomography imaging, positron emission tomography with computed tomography imaging, or positron emission tomography with magnetic resonance imaging. The condition can be a cancer, a liver disease, a neurodegenerative disease, a psychiatric disease, or Alzheimer's disease.

These and other features, aspects, and advantages of the present invention will become better understood upon consideration of the following detailed description, drawings and appended claims.

### BRIEF DESCRIPTION OF THE DRAWINGS

FIG. 1 is a schematic of a positron emission tomography (PET) system.

FIG. 2 shows the designed novel GSK-3 PET probes with their cLogP values.

FIG. 3 shows the Design and synthesis of compounds 4 (¹⁸F-CNBI) and 7 (¹⁸F-CNBIFE) according to the present disclosure.

FIG. 4 shows the Design and synthesis of compound 10 (MC-002) according to the present disclosure for GSK-3 imaging.

FIG. 5 shows the Design and synthesis of compound 13 (MC-003) according to the present disclosure for GSK-3 imaging.

FIG. 6 shows the Design and synthesis of compound 16 (MC-004) according to the present disclosure for GSK-3 imaging.

FIG. 7 shows the Design and synthesis of compound 19 (MC-005) according to the present disclosure for GSK-3 imaging.

FIG. 8 shows the Design and synthesis of compound 22 (MC-006) according to the present disclosure for GSK-3 imaging.

FIG. 9 shows the Design and synthesis of compounds 25 (¹⁸F—CNPI) and 28 (¹⁸F-CNPIFE) according to the present disclosure.

FIG. 10 shows the design and synthesis of a GSK-3 PET probe [¹⁸F]CNPIFE (28) according to the present disclosure.

FIG. 11 shows the Design and synthesis of Boronic acid analogs for making Precursors for compounds 10, 13 and 16 according to the present disclosure for GSK-3 imaging.

FIG. 12 shows the Design and synthesis of Iodo intermediate (compound 1) and Coupling of boronic acid analogs with iodo intermediate for synthesis of Precursors 8, 11 and 14 according to the present disclosure for GSK-3 imaging.

FIG. 13 shows the Coupling of hydroxy boronic acid analogs with iodo intermediate and Alkylation of compound 36 with various alkyl groups which is alternative synthetic method for synthesis of Precursors 8, 11 and 14 according to the present disclosure for GSK-3 imaging.

FIG. 14 shows the Design and synthesis of Boronic acid analogs for making Precursors for compounds 19 and 22 according to the present disclosure for GSK-3 imaging.

FIG. 15 shows the Design and synthesis of Iodo intermediate (compound 1) and Coupling of boronic acid analogs with iodo intermediate for synthesis of Precursors 17 and 20 according to the present disclosure for GSK-3 imaging.

FIG. 16 shows the Coupling of amino boronic acid analogs with iodo intermediate and Alkylation of compound 41 with various alkyl groups which is an alternative synthetic method for synthesis of Precursors 17 and 20 according to the present disclosure for GSK-3 imaging.

FIG. 17 shows the automated radiosynthesis and semi-preparative HPLC purification of [¹⁸F]CNPIFE (28) according to the present disclosure.

FIG. 18 shows the analytical characterization and quality control of a GSK-3 PET probe according to the present disclosure.

FIG. 19 shows a summary of the IC₅₀ value of GSK-3 probes measured using ADP GIo™ Kinase Assay System.

FIG. 20 shows the binding affinity of [¹⁸F]CNPIFE (28) against various tau isoforms and other proteins involved in neurodegeneration.

FIG. 21 shows the uptake (SUV) of [¹⁸F]CNPIFE (28) in a mouse brain over time.

FIG. 22 shows representative Micro-PET Images showing uptake of [¹⁸F]CNPIFE (28) in the mouse brain over time.

FIG. 23 shows a plot of the uptake (SUV) overtime of [¹⁸F]CNPIFE (28) in the mouse brain.

FIG. 24 shows the uptake (SUV) of [¹⁸F]-CNPIFE (28) in the mouse liver over time.

FIG. 25 shows representative micro-PET Images showing uptake (SUV)/biodistribution of [¹⁸F]-CNPIFE (28) in a normal mouse model over time.

FIG. 26 shows blood-brain barrier permeability of compounds [¹⁹F]F-CNBI (2), [¹⁹F]F-9 (9), ([¹⁹F]F—CNPI (23), [¹⁹F]F-CNPIFE (27) and [¹⁹F]F—CNPIOSOF (55) at pH 7.4.

FIG. 27 shows the stability of [¹⁸F]F-CNBI (4) and [¹⁸F]F-CNPIFE (28) in mouse and human serums.

FIG. 28 shows a GSK-3 Imaging Probe [¹⁸F]F-CNPIFEA (51) according to the present disclosure, and the non-radiative reference standard (49) and the precursor (50).

FIG. 29 shows a GSK-3 Imaging Probe [¹⁸F]F-CNPINMFEA (54) according to the present disclosure, and the non-radiative reference standard (52) and the precursor (53).

FIG. 30 shows a GSK-3 Imaging Probe [¹⁸F]F—CNPIOSOF (56) according to the present disclosure, and the non-radiative reference standard (55).

FIG. 31 shows a GSK-3 Imaging Probe [¹⁸F]F-CNPIDF (59) according to the present disclosure, and the non-radiative reference standard (57) and the precursor (58).

FIG. 32 shows a GSK-3 Imaging Probe [¹⁸F]F—CNPI (25) according to the present disclosure, and the non-radiative reference standard (23) and the precursor (60).

FIG. 33 shows a GSK-3 Imaging Probe (63) according to the present disclosure, and the non-radiative reference standard (61) and the precursor (62).

FIG. 34 shows a GSK-3 Imaging Probe (66) according to the present disclosure, and the non-radiative reference standard (64) and the precursor (65).

FIG. 35 shows a GSK-3 Imaging Probe (69) according to the present disclosure, and the non-radiative reference standard (67) and the precursor (68).

FIG. 36 shows a GSK-3 Imaging Probe (72) according to the present disclosure, and the non-radiative reference standard (70) and the precursor (71).

FIG. 37 shows a summary of the IC₅₀ value of compound [¹⁸F]F—CNPIOSOF (56) measured using an enzyme ADP GIo™ assay.

FIG. 38 shows a summary of the IC₅₀ value of compound MC-002 (9) measured using an enzyme ADP GIo™ assay.

FIG. 39 shows a summary of the IC₅₀ value of compound [¹⁸F]F-CNPIFEA (51) measured using an enzyme ADP GIo™ assay.

FIG. 40 shows a summary of the IC₅₀ value of compound [¹⁸F]F-CNPINMFEA (54) measured using an enzyme ADP GIo™ assay.

FIG. 41 shows a summary of the IC₅₀ value of compound [¹⁸F]F-CNPIDF (59) measured using an enzyme ADP GIo™ assay.

FIG. 42 shows IR spectrum confirmation data of compound 9 (MC-002).

FIG. 43 shows ¹H NMR spectrum confirmation data of compound 9 (MC-002).

FIG. 44 shows ¹³C NMR spectrum confirmation data of compound 9 (MC-002).

FIG. 45 shows IR spectrum confirmation data of compound 8 (MC-002 precursor).

FIG. 46 shows ¹H NMR spectrum confirmation data of compound 8 (MC-002 precursor).

FIG. 47 shows ¹³C NMR spectrum confirmation data of compound 8 (MC-002 precursor).

FIG. 48 shows IR spectrum confirmation data of compound 23 (CNPI Standard).

FIG. 49 shows ¹H NMR spectrum confirmation data of compound 23 (CNPI Standard).

FIG. 50 shows ¹³C NMR spectrum confirmation data of compound 23 (CNPI Standard).

FIG. 51 shows a representative HPLC trace of compound 23 (CNPI Standard).

FIG. 52 shows IR spectrum confirmation data of compound 60 (CNPI Precursor).

FIG. 53 shows ¹H NMR spectrum confirmation data of compound 60 (CNPI Precursor).

FIG. 54 shows ¹³C NMR spectrum confirmation data of compound 60 (CNPI Precursor).

FIG. 55 shows IR spectrum confirmation data of compound 49 (CNPIFEA Standard).

FIG. 56 shows ¹H NMR spectrum confirmation data of compound 49 (CNPIFEA Standard).

FIG. 57 shows ¹³C NMR spectrum confirmation data of compound 49 (CNPIFEA Standard).

FIG. 58 shows a representative HPLC trace of compound 49 (CNPIFEA Standard).

FIG. 59 shows IR spectrum confirmation data of compound 50 (CNPIFEA Precursor).

FIG. 60 shows ¹H NMR spectrum confirmation data of compound 50 (CNPIFEA Precursor).

FIG. 61 shows ¹³C NMR spectrum confirmation data of compound 50 (CNPIFEA Precursor).

FIG. 62 shows IR spectrum confirmation data of compound 52 (CNPINMFEA Standard).

FIG. 63 shows ¹H NMR spectrum confirmation data of compound 52 (CNPINMFEA Standard).

FIG. 64 shows ¹³C NMR spectrum confirmation data of compound 52 (CNPINMFEA Standard).

FIG. 65 shows a representative HPLC trace of compound 52 (CNPINMFEA Standard).

FIG. 66 shows IR spectrum confirmation data of compound 53 (CNPINMFEA Precursor).

FIG. 67 shows ¹H NMR spectrum confirmation data of compound 53 (CNPINMFEA Precursor).

FIG. 68 shows ¹³C NMR spectrum confirmation data of compound 53 (CNPINMFEA Precursor).

FIG. 69 shows IR spectrum confirmation data of compound 55 (CNPIOSF Standard).

FIG. 70 shows ¹H NMR spectrum confirmation data of compound 55 (CNPIOSF Standard).

FIG. 71 shows ¹³C NMR spectrum confirmation data of compound 55 (CNPIOSF Standard).

FIG. 72 shows a representative HPLC trace of compound 55 (CNPIOSOF Standard) and [¹⁸F]F—CNPIOSOF.

FIG. 73 shows IR spectrum confirmation data of compound 57 (CNPIDF Standard).

FIG. 74 shows ¹H NMR spectrum confirmation data of compound 57 (CNPIDF Standard).

FIG. 75 shows ¹³C NMR spectrum confirmation data of compound 57 (CNPIDF Standard).

FIG. 76 shows ¹H NMR spectrum confirmation data of compound 3 (CNBI Precursor).

FIG. 77 shows ¹³C NMR spectrum confirmation data of compound 3 (CNBI Precursor).

FIG. 78 shows ¹H-¹H-COSY spectrum confirmation data of compound 3 (CNBI Precursor).

FIG. 79 shows ¹H-¹³C-COSY spectrum confirmation data of compound 3 (CNBI Precursor).

FIG. 80 shows ¹H NMR spectrum confirmation data of compound 2 (CNBI Standard).

FIG. 81 shows ¹³C NMR spectrum confirmation data of compound 2 (CNBI Standard).

FIG. 82 shows ¹⁹F NMR spectrum confirmation data of compound 2 (CNBI Standard).

FIG. 83 shows ¹H-¹H-COSY spectrum confirmation data of compound 2 (CNBI Standard).

FIG. 84 shows ¹H-¹³C-COSY spectrum confirmation data of compound 2 (CNBI Standard).

FIG. 85 shows ¹H NMR spectrum confirmation data of compound 26 (CNPIFE Precursor).

FIG. 86 shows ¹³C NMR spectrum confirmation data of compound 26 (CNPIFE Precursor).

FIG. 87 shows ¹H-¹H-COSY spectrum confirmation data of compound 26 (CNPIFE Precursor).

FIG. 88 shows ¹H-¹³C-COSY spectrum confirmation data of compound 26 (CNPIFE Precursor).

FIG. 89 shows ¹H NMR spectrum confirmation data of compound 27 (CNPIFE Standard).

FIG. 90 shows ¹³C NMR spectrum confirmation data of compound 27 (CNPIFE Standard).

FIG. 91 shows ¹⁹F NMR spectrum confirmation data of compound 27 (CNPIFE Standard).

FIG. 92 shows ¹H-¹H-COSY spectrum confirmation data of compound 27 (CNPIFE Standard).

FIG. 93 shows ¹H-¹³C-COSY spectrum confirmation data of compound 27 (CNPIFE Standard).

FIG. 94 shows r-TLC data of compound 28 ([¹⁸F]F-CNPIFE) and compound 4 ([¹⁸F]F-CNBI).

FIG. 95 shows a representative HPLC trace of compound 28 ([¹⁸F]F-CNPIFE) and [¹⁹F]F-CNPIFE.

FIG. 96 shows a representative HPLC trace of compound 4 ([¹⁸F]F-CNBI) and [¹⁹F]F-CNBI.

### DETAILED DESCRIPTION OF THE INVENTION

The present disclosure shows the feasibility of novel BBB permeable and selective probes for GSK-3 using PET in a preclinical GSK-3 tau mouse model of Alzheimer's disease (AD). We have successfully synthesized ¹⁸F-labeled PET probes, evaluated their binding affinity towards GSK-3α and GSK-3β, and also with other competing proteins commonly associated with AD pathology. We have also performed an in vivo biodistribution (pharmacokinetics) using micro-PET imaging in a normal mouse model. The developed PET probes are extremely promising as they showed high nanomolar affinity towards both GSK-3α and GSK-3β, and no binding towards other competing proteins. The successful translation of the PET probes of the present disclosure to clinic will help to better understand the role of GSK-3 on AD onset and progression. The developed PET probes will help to diagnose AD much earlier than currently available technique(s) and it will also help to advance the treatment of AD with next generation of GSK-3 inhibitors.

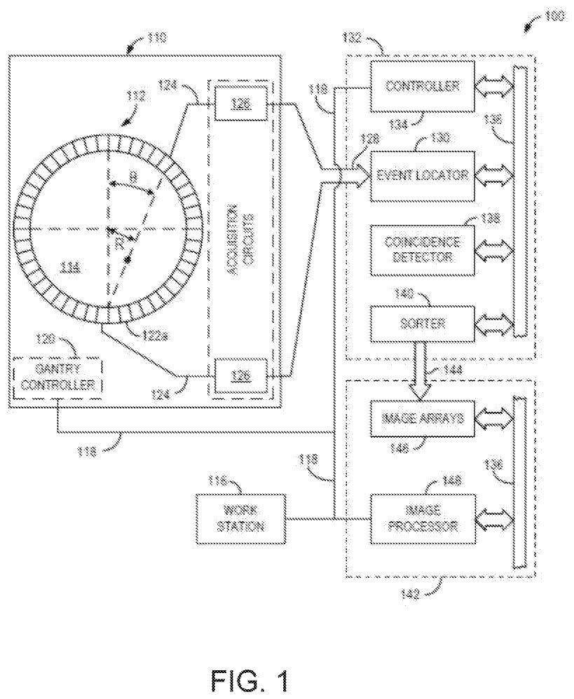

Referring now to FIG. 1, a PET system 100 that can be used with a PET probe of the present invention comprises an imaging hardware system 110 that includes a detector ring assembly 112 about a central axis or bore 114. An operator workstation 116 including a commercially available processor running a commercially available operating system communicates through a communications link 118 with a gantry controller 120 to control operation of the imaging hardware system 110.

The detector ring assembly 112 is formed of a multitude of radiation detector units 122 that produce a signal responsive to detection of a photon on communications line 124 when an event occurs. A set of acquisition circuits 126 receive the signals and produce signals indicating the event coordinates (x, y) and the total energy associated with the photons that caused the event. These signals are sent through a cable 128 to an event locator circuit 130. Each acquisition circuit 126 also produces an event detection pulse that indicates the exact moment the interaction took place. Other systems utilize sophisticated digital electronics that can also obtain this information regarding the precise instant in which the event occurred from the same signals used to obtain energy and event coordinates.

The event locator circuits 130 in some implementations, form part of a data acquisition processing system 132 that periodically samples the signals produced by the acquisition circuits 126. The data acquisition processing system 132 includes a general controller 134 that controls communications on a backplane bus 136 and on the general communications network 118. The event locator circuits 130 assemble the information regarding each valid event into a set of numbers that indicate precisely when the event took place and the position in which the event was detected. This event data packet is conveyed to a coincidence detector 138 that is also part of the data acquisition processing system 132.

The coincidence detector 138 accepts the event data packets from the event locator circuit 130 and determines if any two of them are in coincidence. Coincidence is determined by a number of factors. First, the time markers in each event data packet must be within a predetermined time window, for example, 0.5 nanoseconds or even down to picoseconds. Second, the locations indicated by the two event data packets must lie on a straight line that passes through the field of view in the scanner bore 114. Events that cannot be paired are discarded from consideration by the coincidence detector 138, but coincident event pairs are located and recorded as a coincidence data packet. These coincidence data packets are provided to a sorter 140. The function of the sorter in many traditional PET imaging systems is to receive the coincidence data packets and generate memory addresses from the coincidence data packets for the efficient storage of the coincidence data. In that context, the set of all projection rays that point in the same direction (θ) and pass through the scanner's field of view (FOV) is a complete projection, or “view”. The distance (R) between a particular projection ray and the center of the FOV locates that projection ray within the FOV. The sorter 140 counts all of the events that occur on a given projection ray (R, θ) during the scan by sorting out the coincidence data packets that indicate an event at the two detectors lying on this projection ray. The coincidence counts are organized, for example, as a set of two-dimensional arrays, one for each axial image plane, and each having as one of its dimensions the projection angle θ and the other dimension the distance R. This θ by R map of the measured events is call a histogram or, more commonly, a sinogram array. It is these sinograms that are processed to reconstruct images that indicate the number of events that took place at each image pixel location during the scan. The sorter 140 counts all events occurring along each projection ray (R, θ) and organizes them into an image data array.

The sorter 140 provides image datasets to an image processing/reconstruction system 142, for example, by way of a communications link 144 to be stored in an image array 146. The image arrays 146 hold the respective datasets for access by an image processor 148 that reconstructs images. The image processing/reconstruction system 142 may communicate with and/or be integrated with the work station 116 or other remote work stations.

The PET system 100 provides an example emission tomography system for acquiring a series of medical images of a subject during an imaging process after administering a pharmaceutically acceptable composition including a PET probe as described herein. The system includes a plurality of detectors configured to be arranged about the subject to acquire gamma rays emitted from the subject over a time period relative to an administration of the composition to the subject and communicate signals corresponding to acquired gamma rays. The system also includes a reconstruction system configured to receive the signals and reconstruct therefrom a series of medical images of the subject. In one version of the system, a second series of medical images is concurrently acquired using an x-ray computed tomography imaging device. In one version of the system, a second series of medical images is concurrently acquired using a magnetic resonance imaging device.

Administration to the subject of a pharmaceutical composition including a PET probe of the invention can be accomplished intravenously, intraarterially, intrathecally, intramuscularly, intradermally, subcutaneously, intraperitoneally or intracavitary. A “subject” is a mammal, preferably a human. In the method of the invention, sufficient time is allowed after administration of a detectable amount of the PET probe of the invention such that the PET probe can accumulate in a target region of the subject. A “detectable amount” means that the amount of the PET probe that is administered is sufficient to enable detection of accumulation of the PET probe in a subject by a medical imaging technique.

One non-limiting example method of imaging according to the invention involves the use of an intravenous injectable composition including a PET probe of the invention. A positron emitting atom of the PET probe gives off a positron, which subsequently annihilates and gives off coincident gamma radiation. This high energy gamma radiation is detectable outside the body using positron emission tomography imaging, or positron emission tomography concurrent with computed tomography imaging (PET/CT), or positron emission tomography with magnetic resonance imaging (PET/MRI). With PET/CT, the location of the injected and subsequently accumulated PET probe within the body can be identified.

In one embodiment, the present invention provides a compound of formula (1):

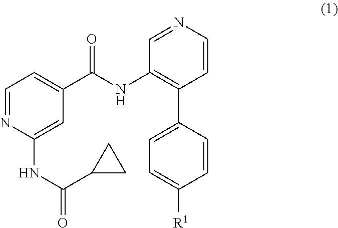

wherein R¹ is selected from the group consisting of unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, substituted alkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R¹ is replaced with a positron emitter. In one version of the compound, the positron emitter is selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

One embodiment of the compound has the formula (28A):

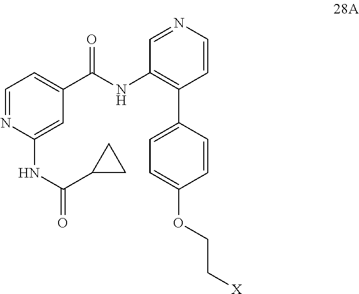

wherein X is a positron emitter selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

Another embodiment of the compound has the formula (28):

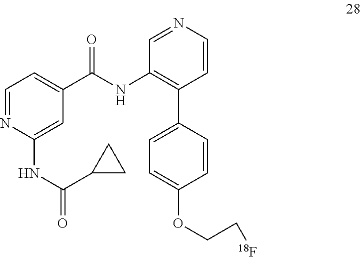 [SMILES: O=C(Nc1cnccc1-c1ccc(OCC[18F])cc1)c1ccnc(NC(=O)C2CC2)c1]

Another embodiment of the compound has the formula (51):

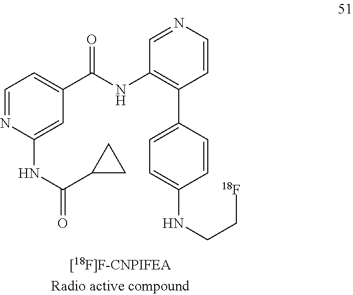 [SMILES: O=C(Nc1cnccc1-c1ccc(NCC[18F])cc1)c1ccnc(NC(=O)C2CC2)c1]

Another embodiment of the compound has the formula (54):

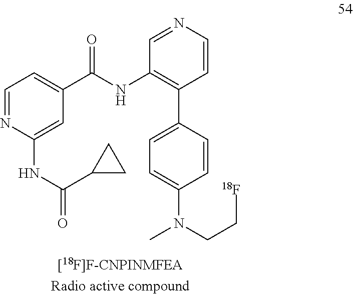 [SMILES: CN(CC[18F])c1ccc(-c2ccncc2NC(=O)c2ccnc(NC(=O)C3CC3)c2)cc1]

Another embodiment of the compound has the formula (56):

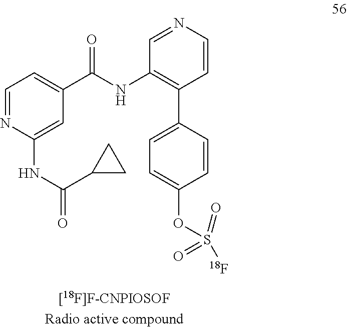 [SMILES: O=C(Nc1cnccc1-c1ccc(OS(=O)(=O)[18F])cc1)c1ccnc(NC(=O)C2CC2)c1]

The compounds of formula (1), formula (28A), formula (28), formula (51), formula (54) and formula (56) can be capable of binding to GSK-3. The compounds of formula (1), formula (28A), formula (28), formula (51), formula (54) and formula (56) can be capable of specific binding to GSK-3. The compounds of formula (1), formula (28A), formula (28), formula (51), formula (54) and formula (56) can be capable of specific binding to GSK-3α and GSK-3β. The compounds of formula (1), formula (28A), formula (28), formula (51), formula (54) and formula (56) can be capable of not binding with proteins that compete with GSK-3α and GSK-3β. The compounds of formula (1), formula (28A), formula (28), formula (51), formula (54) and formula (56) can exhibit blood brain barrier (BBB) penetration. The compounds of formula (1), formula (28A), formula (28), formula (51), formula (54) and formula (56) inhibit GSK-3.

In one embodiment, the present invention provides a compound of formula (2):

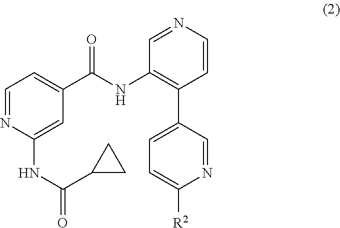

wherein R² is selected from the group consisting of unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R¹ is replaced with a positron emitter. In one version of the compound, R² is unsubstituted alkoxy, and the positron emitter is selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

One embodiment of the compound has the formula (7a):

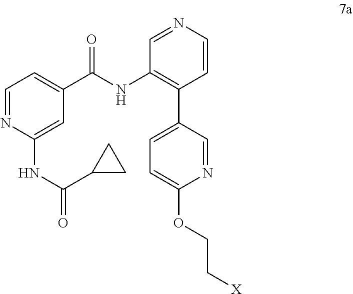

wherein X is a positron emitter selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y, ⁸⁹Zr, ⁹⁰Nb, ^{94m}Tc, ^{110m}In, ¹¹⁸Sb, ¹²⁰I, ¹²¹I, ¹²²I, and ¹²⁴I.

One embodiment of the compound has the formula (7):

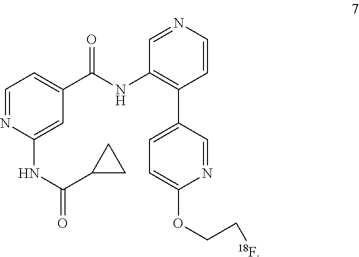

One embodiment of the compound has the formula MC-002 (10):

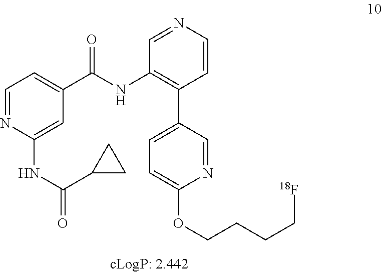 [SMILES: O=C(Nc1cnccc1-c1ccc(OCCCC[18F])nc1)c1ccnc(NC(=O)C2CC2)c1]

One embodiment of the compound has the formula MC-003 (13):

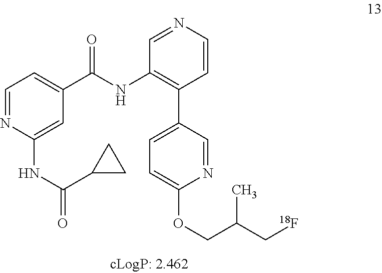 [SMILES: CC(C[18F])COc1ccc(-c2ccncc2NC(=O)c2ccnc(NC(=O)C3CC3)c2)cn1]

One embodiment of the compound has the formula MC-004 (16):

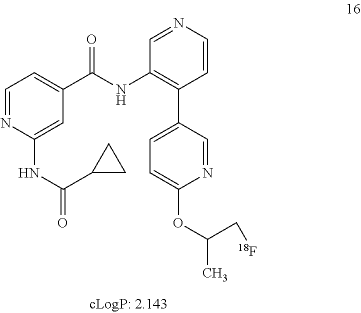 [SMILES: CC(C[18F])Oc1ccc(-c2ccncc2NC(=O)c2ccnc(NC(=O)C3CC3)c2)cn1]

One embodiment of the compound has the formula MC-005 (19):

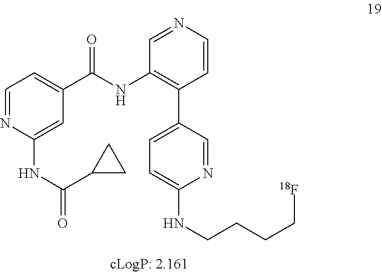 [SMILES: O=C(Nc1cnccc1-c1ccc(NCCCC[18F])nc1)c1ccnc(NC(=O)C2CC2)c1]

One embodiment of the compound has the formula MC-006 (22):

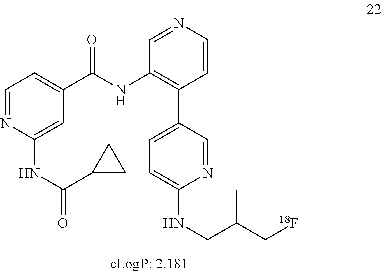 [SMILES: CC(C[18F])CNc1ccc(-c2ccncc2NC(=O)c2ccnc(NC(=O)C3CC3)c2)cn1]

The compounds of formula (2), formula (7a), formula (7), formula (10), formula (13), formula (16), formula (19), and formula (22) can be capable of binding to GSK-3. The compounds of formula (2), formula (7a), formula (7), formula (10), formula (13), formula (16), formula (19), and formula (22) can be capable of specific binding to GSK-3. The compounds of formula (2), formula (7a), formula (7), formula (10), formula (13), formula (16), formula (19), and formula (22) can be capable of specific binding to GSK-3α and GSK-3β. The compounds of formula (2), formula (7a), formula (7), formula (10), formula (13), formula (16), formula (19), and formula (22) can be capable of not binding with proteins that compete with GSK-3α and GSK-3β. The compounds of formula (2), formula (7a), formula (7), formula (10), formula (13), formula (16), formula (19), and formula (22) can exhibit blood brain barrier (BBB) penetration. The compounds of formula (2), formula (7a), formula (7), formula (10), formula (13), formula (16), formula (19), and formula (22) can inhibit GSK-3.

In one embodiment, the present invention provides a compound of formula (75):

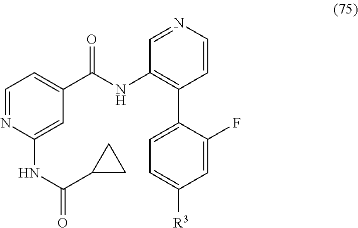

wherein R³ is selected from the group consisting of hydrogen, unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R³ is replaced with a positron emitter. In one version of the compound, the positron emitter is selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y ⁸⁹Zr, ⁹⁰Nb, ^{94m}Tc, ^{110m}In, ¹¹⁸Sb, ¹²⁰I, ¹²¹I, ¹²²I, and ¹²⁴I.

One embodiment of the compound has the formula (59):

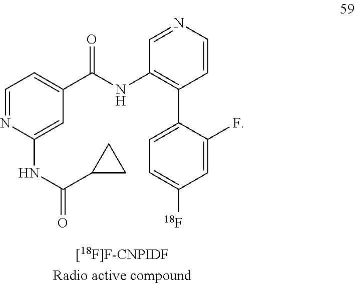 [SMILES: O=C(Nc1cnccc1-c1ccc([18F])cc1F)c1ccnc(NC(=O)C2CC2)c1]

The compounds of formula (75) and formula (59) can be capable of binding to GSK-3. The compounds of formula (75) and formula (59) can be capable of specific binding to GSK-3. The compounds of formula (75) and formula (59) can be capable of specific binding to GSK-3α and GSK-3β. The compounds of formula (75) and formula (59) can be capable of not binding with proteins that compete with GSK-3a and GSK-3β. The compounds of formula (75) and formula (59) can exhibit blood brain barrier (BBB) penetration. The compounds of formula (75) and formula (59) can inhibit GSK-3.

In one embodiment, the present invention provides a compound of formula (80):

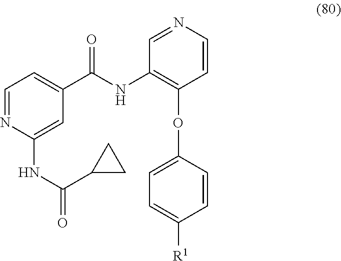

wherein R⁴ is selected from the group consisting of hydrogen, unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R⁴ is replaced with a positron emitter. In one version of the compound, the positron emitter is selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y ⁸⁹Zr, ⁹⁰Nb, ^{94m}Tc, ^{110m}In, ¹¹⁸Sb, ¹²⁰I, ¹²¹I, ¹²²I, and ¹²⁴I.

One embodiment of the compound has the formula (63):

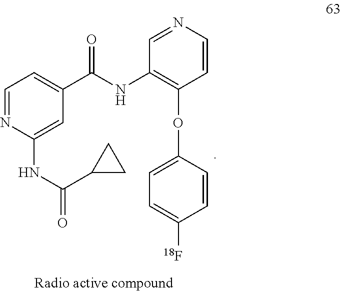 [SMILES: O=C(Nc1cnccc1Oc1ccc([18F])cc1)c1ccnc(NC(=O)C2CC2)c1]

One embodiment of the compound has the formula (66):

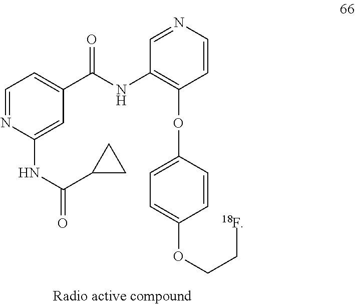

The compounds of formula (80) and formula (63) and formula (66) can be capable of binding to GSK-3. The compounds of formula (80) and formula (63) and formula (66) can be capable of specific binding to GSK-3. The compounds of formula (80) and formula (63) and formula (66) can be capable of specific binding to GSK-3α and GSK-3β. The compounds of formula (80) and formula (63) and formula (66) can be capable of not binding with proteins that compete with GSK-3α and GSK-3β. The compounds of formula (80) and formula (63) and formula (66) can exhibit blood brain barrier (BBB) penetration. The compounds of formula (80) and formula (63) and formula (66) can inhibit GSK-3.

In one embodiment, the present invention provides a compound of formula (90):

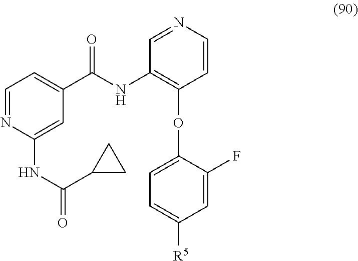

wherein R⁵ is selected from the group consisting of hydrogen, unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and wherein at least one atom in R⁵ is replaced with a positron emitter. In one version of the compound, the positron emitter is selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y ⁸⁹Zr, ⁹⁰Nb, ^{94m}Tc, ^{110m}In, ¹¹⁸Sb, ¹²⁰I, ¹²¹I, ¹²²I, and ¹²⁴I.

One embodiment of the compound has the formula (69):

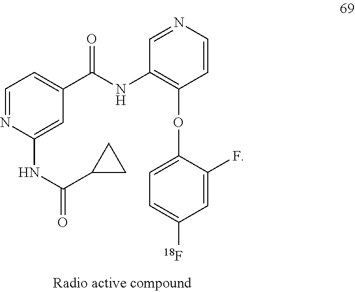 [SMILES: O=C(Nc1cnccc1Oc1ccc([18F])cc1F)c1ccnc(NC(=O)C2CC2)c1]

One embodiment of the compound has the formula (72):

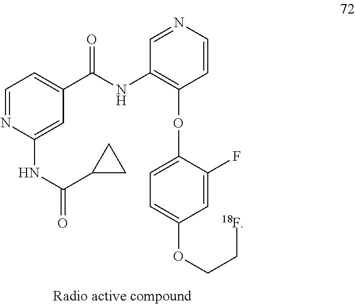

The compounds of formula (90) and formula (69) and formula (72) can be capable of binding to GSK-3. The compounds of formula (90) and formula (69) and formula (72) can be capable of specific binding to GSK-3. The compounds of formula (90) and formula (69) and formula (72) can be capable of specific binding to GSK-3α and GSK-3β. The compounds of formula (90) and formula (69) and formula (72) can be capable of not binding with proteins that compete with GSK-3α and GSK-3β. The compounds of formula (90) and formula (69) and formula (72) can exhibit blood brain barrier (BBB) penetration. The compounds of formula (90) and formula (69) and formula (72) can inhibit GSK-3.

The present disclosure also provides a method for in vivo imaging of a subject, wherein the method comprises: (a) administering to the subject the compound of formula (1), formula (2), formula (75), formula (80), formula (90), formula (7a), formula (7), formula (10), formula (13), formula (16), formula (19), formula (22), formula (5), formula (7), formula (51), formula (54), formula (56), formula (59), formula (63), formula (66), formula (69), or formula (72); (b) waiting a time sufficient to allow the compound to accumulate at a tissue or cell site to be imaged; and (c) imaging the cells or tissues with a non-invasive imaging technique. The non-invasive imaging technique can be selected from positron emission tomography imaging, positron emission tomography with computed tomography imaging, or positron emission tomography with magnetic resonance imaging.

The present disclosure also provides a method for detecting GSK-3 in a subject, wherein the method comprises: (a) administering to the subject the compound of formula (1), formula (2), formula (75), formula (80), formula (90), formula (7a), formula (7), formula (10), formula (13), formula (16), formula (19), formula (22), formula (5), formula (7), formula (51), formula (54), formula (56), formula (59), formula (63), formula (66), formula (69), or formula (72), (b) waiting a time sufficient to allow the compound to accumulate at a tissue or cell site to be imaged; and (c) imaging the cells or tissues with a non-invasive imaging technique.

The non-invasive imaging technique can be selected from positron emission tomography imaging, positron emission tomography with computed tomography imaging, or positron emission tomography with magnetic resonance imaging.

The present disclosure also provides a method for the treatment of a condition involving GSK-3 activity, wherein the method comprises: administering to a subject having the condition a therapeutically effective amount of the compound of formula (1), formula (2), formula (75), formula (80), formula (90), formula (7a), formula (7), formula (10), formula (13), formula (16), formula (19), formula (22), formula (5), formula (7), formula (51), formula (54), formula (56), formula (59), formula (63), formula (66), formula (69), or formula (72), The condition can be a cancer, a liver disease, a neurodegenerative disease, a psychiatric disease, or Alzheimer's disease.

The present disclosure also provides a method for detecting or ruling out a condition involving GSK-3 activity in a subject, wherein the method comprises: (a) administering to a subject a detectable amount of the compound of formula (1), formula (2), formula (75), formula (80), formula (90), formula (7a), formula (7), formula (10), formula (13), formula (16), formula (19), formula (22), formula (5), formula (7), formula (51), formula (54), formula (56), formula (59), formula (63), formula (66), formula (69), or formula (72), wherein the compound is targeted to GSK-3 at a tissue or cell site in the subject; and (b) acquiring an image of the cells or tissues to detect the presence or absence of GSK-3 in the subject. The image can be acquired using a non-invasive imaging technique selected from positron emission tomography imaging, positron emission tomography with computed tomography imaging, or positron emission tomography with magnetic resonance imaging. The condition can be a cancer, a liver disease, a neurodegenerative disease, a psychiatric disease, or Alzheimer's disease.

### EXAMPLES

The following Examples are provided in order to demonstrate and further illustrate certain embodiments and aspects of the present invention and are not to be construed as limiting the scope of the invention.

### Example 1

2-(cyclopropanecarboxamido)-N-(4-(4-¹⁸F-fluorophenyl)pyridin-3-yl)isonicotinamide [¹⁸F]—CNPI (25) (cLogP: 2.286) and 2-(cyclopropanecarboxamido)-N-(6-(fluoro-¹⁸F)-[3,4′-bipyridin]-3′-yl)isonicotinamide [¹⁸F]—CNBI (4) (cLogP: 0.956), were synthesized using their respective nitro precursors, 2-(cyclopropanecarboxamido)-N-(4-(4-nitrophenyl)pyridin-3-yl)isonicotinamide and 2-(cyclopropanecarboxamido)-N-(6-(nitro)-[3,4′-bipyridin]-3′-yl)isonicotinamide. A standard, nucleophilic reaction using cryptand (Kryptofix/K222, 8.1 mg), potassium carbonate (K₂CO₃, 4.0 mg), and ¹⁸F-fluoride was employed for the ¹⁸F-labeling. The labeling was performed at 165° C., 30 minutes in anhydrous DMSO using 5 mg of nitro precursor and 4 mg of 18-crown-6 as a catalyst. Final products were purified using an Oasis HLB Sep-Pak and concentrated through a standard C-18 plus solid phase extraction cartridge via trap and release. The identities of the synthesized PET probes [¹⁸F]—CNPI (7) and [¹⁸F]—CNBI (4) were confirmed using their respective non-radiative reference standards ¹⁹F—CNPI and ¹⁹F-CNBI on an analytical HPLC. All the radiosyntheses were carried out manually. [¹⁸F]—CNBI (4) and [¹⁸F]—CNPI (25) are shown below.

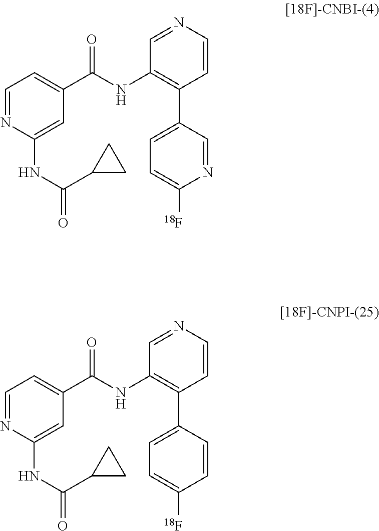 [SMILES: O=C(Nc1cnccc1-c1ccc([18F])nc1)c1ccnc(NC(=O)C2CC2)c1 ; O=C(Nc1cnccc1-c1ccc([18F])cc1)c1ccnc(NC(=O)C2CC2)c1]

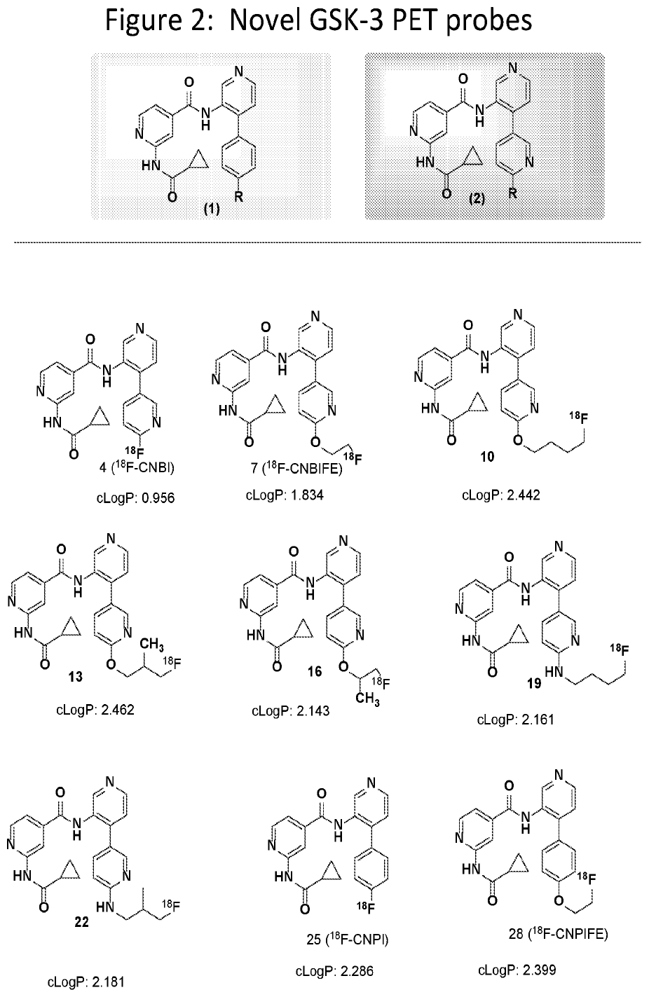

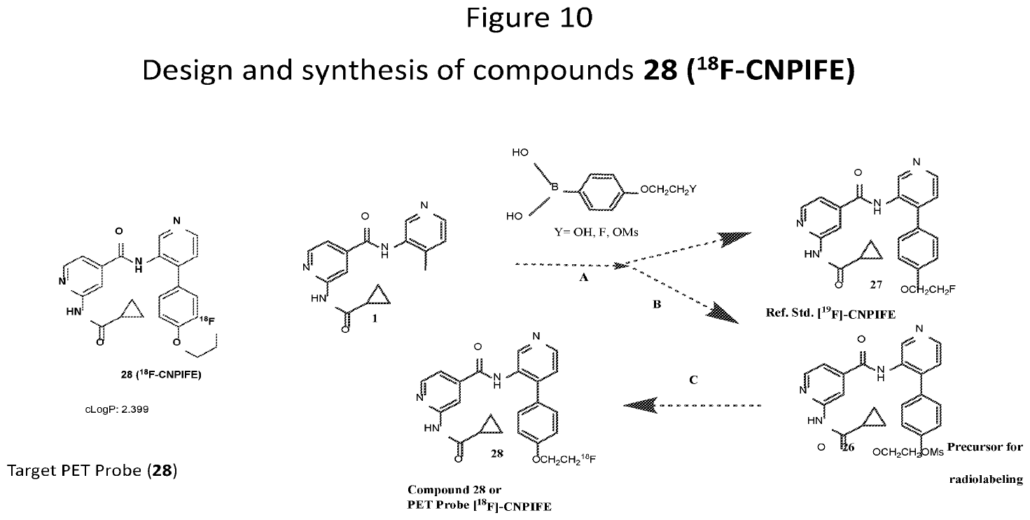

A GSK-3 PET probe [¹⁸F]CNPIFE (28) was synthesized using the scheme shown in FIG. 10. A GSK-3 PET probe [¹⁸F]CNBIFE (7) was synthesized using the scheme shown in FIG. 2 except that

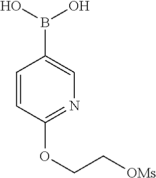 [SMILES: CS(=O)(=O)OCCOc1ccc(B(O)O)cn1]

was used in place of

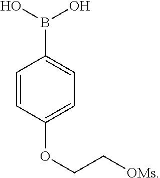 [SMILES: CS(=O)(=O)OCCOc1ccc(B(O)O)cc1]

[¹⁸F]CNPIFE (28) (cLogP: 2.399) and [¹⁸F]CNBIFE (7) (cLogP: 1.834) are shown below.

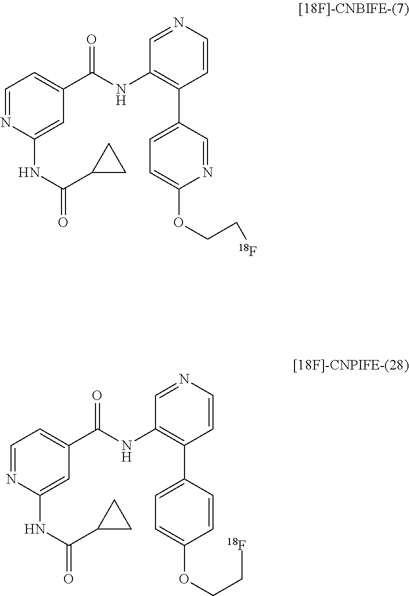 [SMILES: O=C(Nc1cnccc1-c1ccc(OCC[18F])nc1)c1ccnc(NC(=O)C2CC2)c1 ; O=C(Nc1cnccc1-c1ccc(OCC[18F])cc1)c1ccnc(NC(=O)C2CC2)c1]

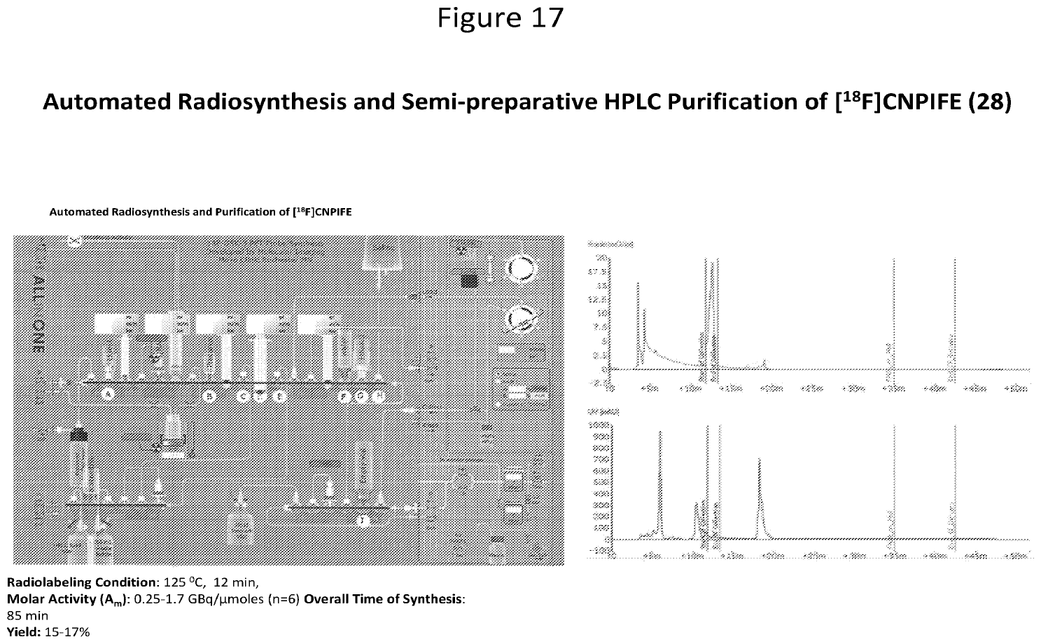

Automation and Radiolabeling of [¹⁸F]-CNBIFE (7) and [¹⁸F]-CNPIFE (28): To minimize the radiation exposure during routine production of [¹⁸F]-CNBIFE (7) and [¹⁸F]-CNPIFE (28), an automated radiosynthesis method was developed using an all-in-one radiosynthetic module equipped with a semi preparative HPLC system. Automation involves sequence development for synthesis, purification, formulation. The automated method was tested and a method validation was performed. FIG. 17 shows the automated radiosynthesis and semi-preparative HPLC purification of [¹⁸F]CNPIFE (28) for this Example 1. The radiolabeling conditions were 125° C. and 12 minutes. The molar activity (Aₘ) was 0.25-1.7 GBq/μmoles (n=6). The overall time of synthesis was 85 minutes. The yield was 15-17%.

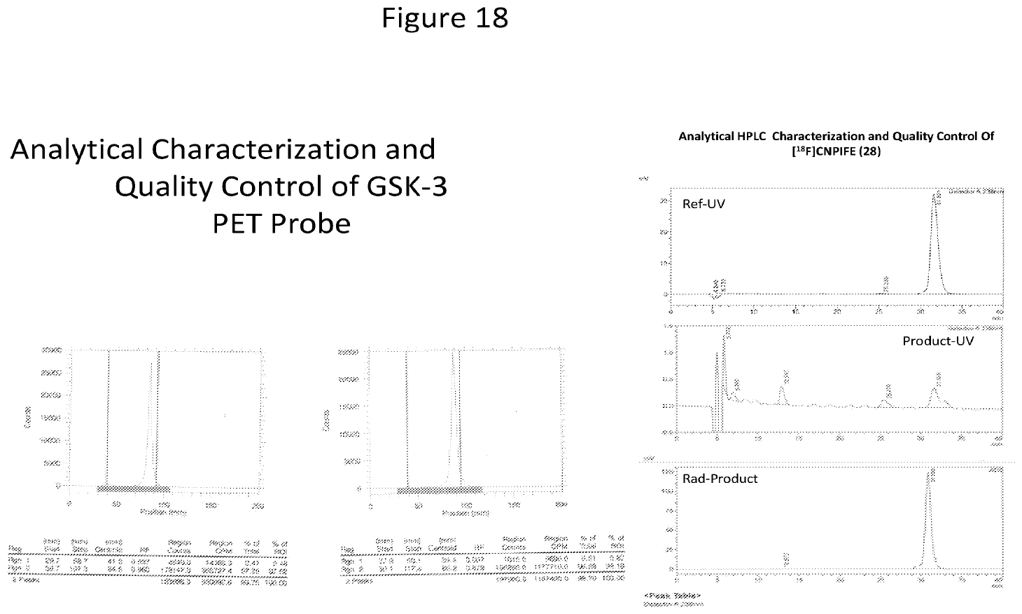

FIG. 18 shows the analytical characterization and quality control of [¹⁸F]CNPIFE (28) for this Example 1.

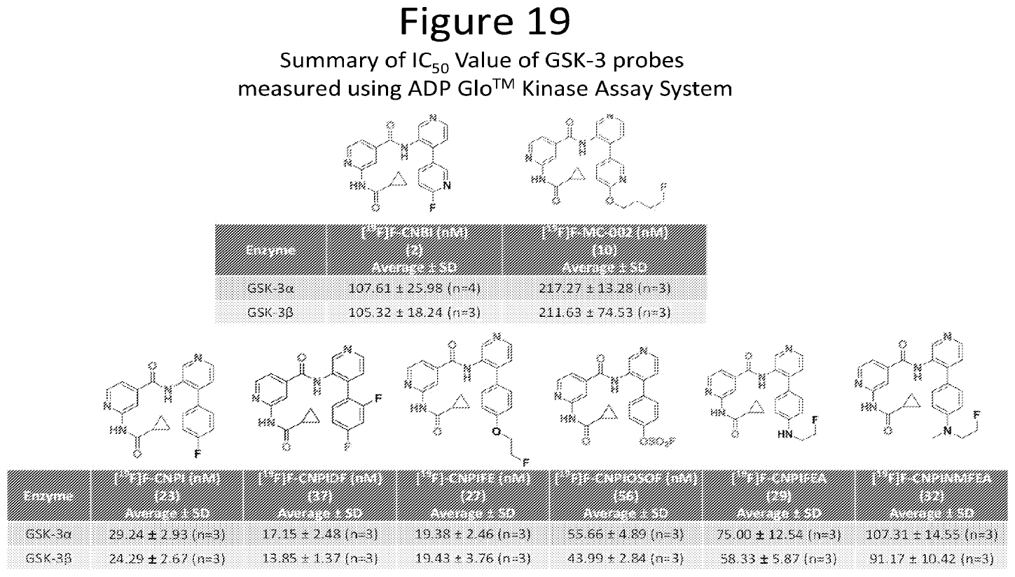

The inhibitory effect of [¹⁸F]—CNBI (4) and [¹⁸F]—CNPI (25) (as synthesized as described above) and [¹⁸F]CNPIFE (28) (as synthesized as shown in FIG. 10) on kinase activity was measured. FIG. 19 shows a summary of the IC₅₀ value of [¹⁸F]—CNBI (4) and [¹⁸F]—CNPI (25) and [¹⁸F]CNPIFE (28) for GSK-3α and GSK-33 measured using an enzyme ADP GIo™ assay. Determination of IC₅₀ was determined as follows. The phosphorylation of glycogen synthase substrate by human glycogen synthase kinase α (GSK-3α) or human glycogen synthase kinase β (GSK-3β) was performed using human GSK-3α/β Kinase Enzyme Assay System (Promega Corporation, Madison, WI) in the presence of GSK-3α/β probes (0-400 nM). The phosphorylation of glycogen synthase substrate was quantified using ADP-Glo™ Kinase assay system (Promega Corporation, Madison, WI). IC₅₀ of GSK-3α/β probes was determined from resulting dose response curve and IC₅₀ curve fitting tool of GraphPad Prism 10 (GraphPad Software, San Diego, CA). (See Stein et al., “Comparison of [¹⁸F]F-CNBI and [¹⁸F]F-CNPIFE as Positron Emission Tomography Probes for Noninvasive Imaging of Glycogen Synthase Kinase-3 in Normal Mice”, Eur. J. Org. Chem. 2022).

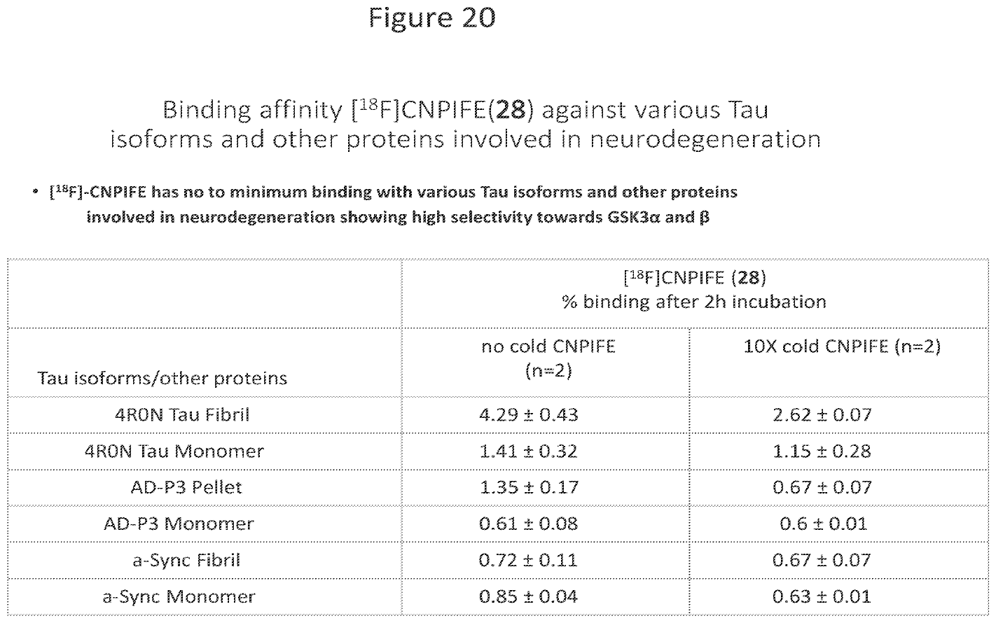

The affinity of [¹⁸F]CNPIFE (28) towards various tau isoforms and other proteins involved in neurodegeneration was measured. FIG. 20 shows the binding affinity of [¹⁸F]CNPIFE (28) against various tau isoforms and other proteins involved in neurodegeneration. [¹⁸F]CNPIFE (28) has no to minimum binding with various tau isoforms and other proteins involved in neurodegeneration showing high selectivity towards GSK-3α and GSK-3β.

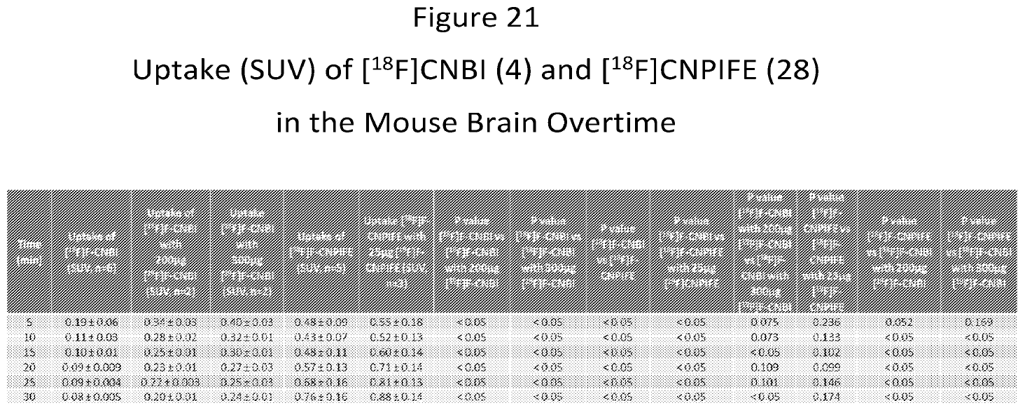

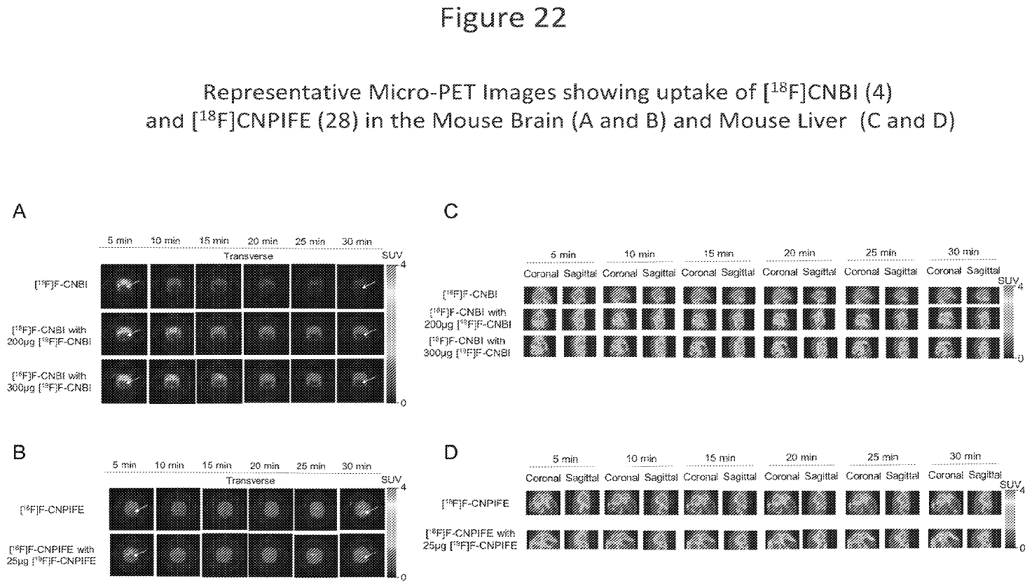

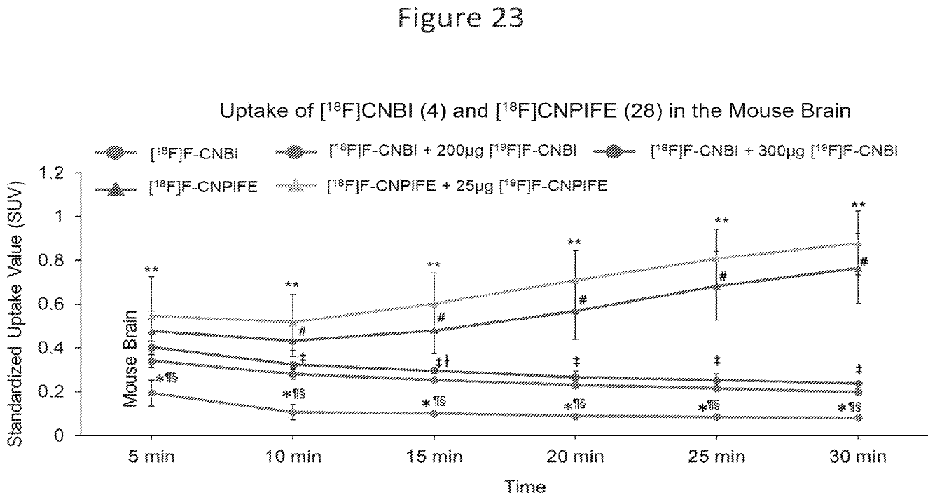

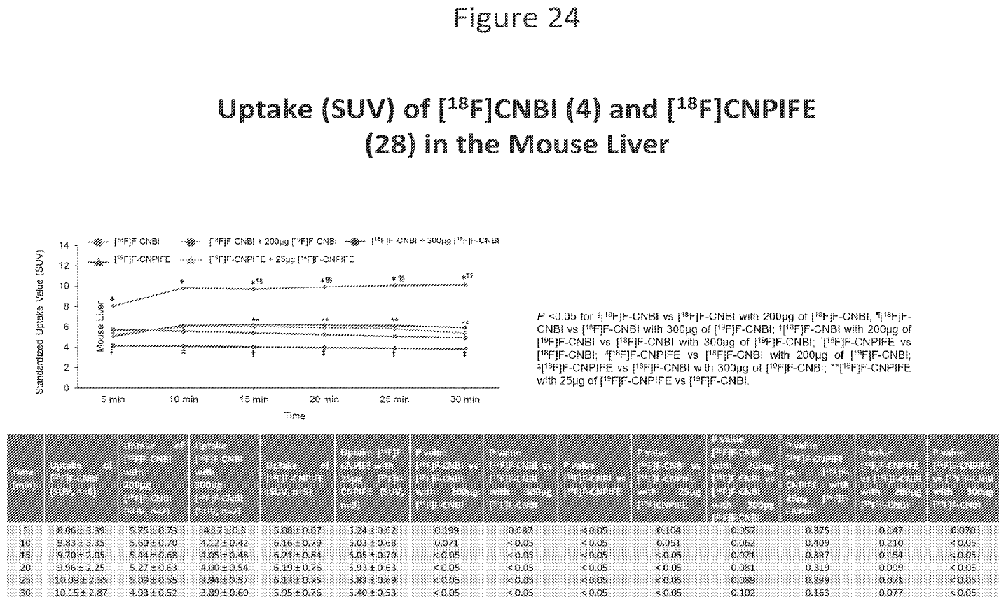

Initial in vivo study in mouse model: Initial in vivo PET imaging studies were conducted in mice at different time points. The uptake of the [¹⁸F]CNPIFE (28) probe is expressed as a standard uptake value (SUV). FIG. 21 shows the SUV of [¹⁸F]CNPIFE (28) in a mouse brain over time. FIG. 22 shows representative micro-PET images showing uptake of [¹⁸F]CNPIFE (28) in the mouse brain. FIG. 23 shows a plot of the SUV of [¹⁸F]CNPIFE (28) in the mouse brain. FIG. 24 shows the SUV of [¹⁸F]-CNPIFE (28) in the mouse liver over time.

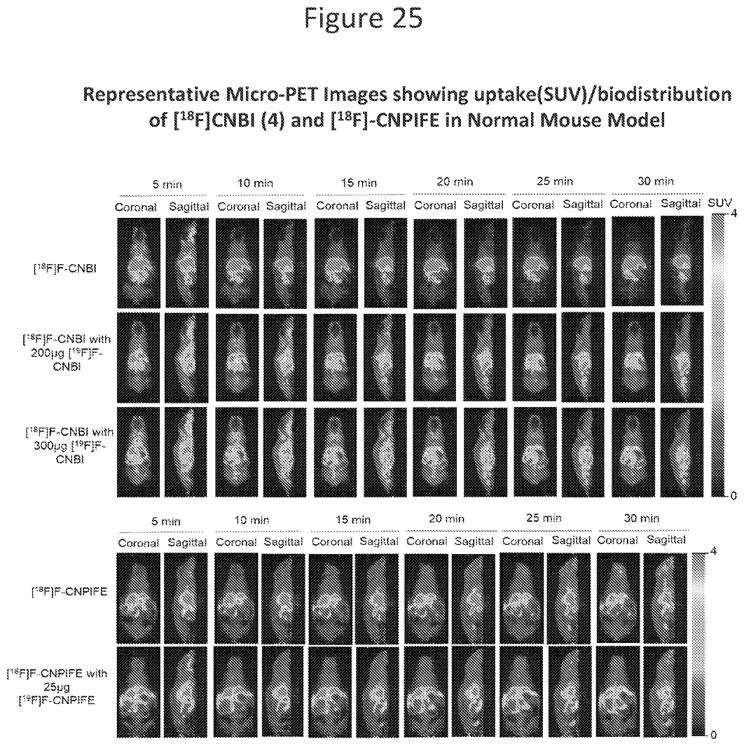

Biodistribution Study: A biodistribution study was undertaken. FIG. 25 shows representative micro-PET Images showing uptake (SUV)/biodistribution of [¹⁸F]-CNPIFE (28) in a normal mouse model.

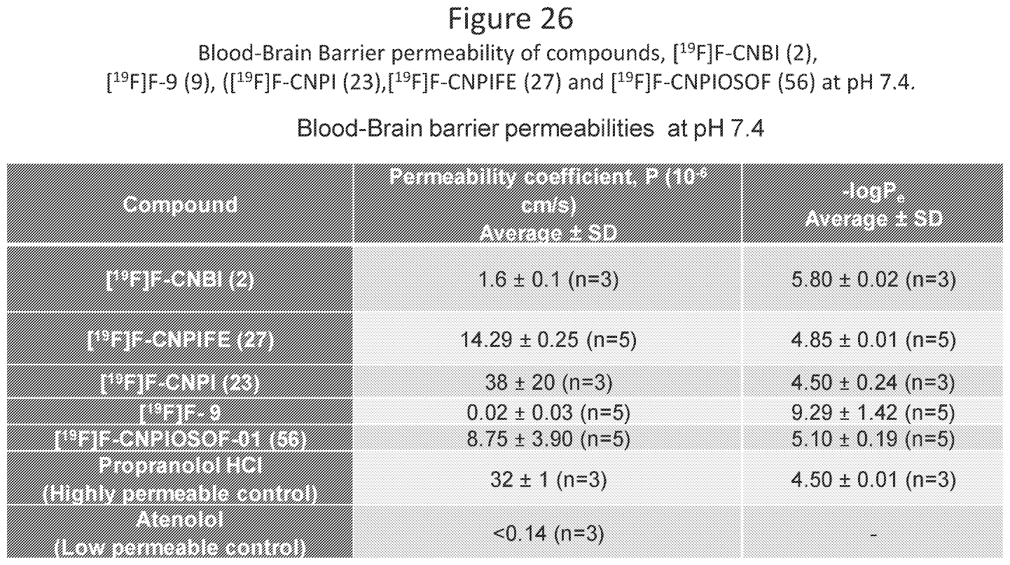

In vivo study in mouse model: In vivo PET imaging studies were conducted in mice at different time points to compare the SUV for [¹⁸F]—CNBI (4) and [¹⁸F]CNPIFE (28). FIG. 26 shows a comparison of the uptake (SUV) of [¹⁸F]—CNBI (4) and [¹⁸F]CNPIFE (28) in a mouse brain. [¹⁸F]CNPIFE (28) showed significantly higher uptake (0.9 SUV) in the brain than [¹⁸F]CNBI (4) (0.4 SUV) confirming a high blood brain barrier permeability.

It can be seen that [¹⁸F]CNPIFE (28) has better purity (about 99% purity), better binding affinity (6×), and better blood brain barrier permeability (6×) compared to previous compounds such as [¹⁸F]CNBI (4). The standard uptake is very close to 1, and to the gold standard value for [N-Methyl-¹¹C]2-(4′-methylaminophenyl)-6-hydroxybenzothiazole also known as [¹¹C] Pittsburgh compound B or [¹¹C]PiB.

### Example 2

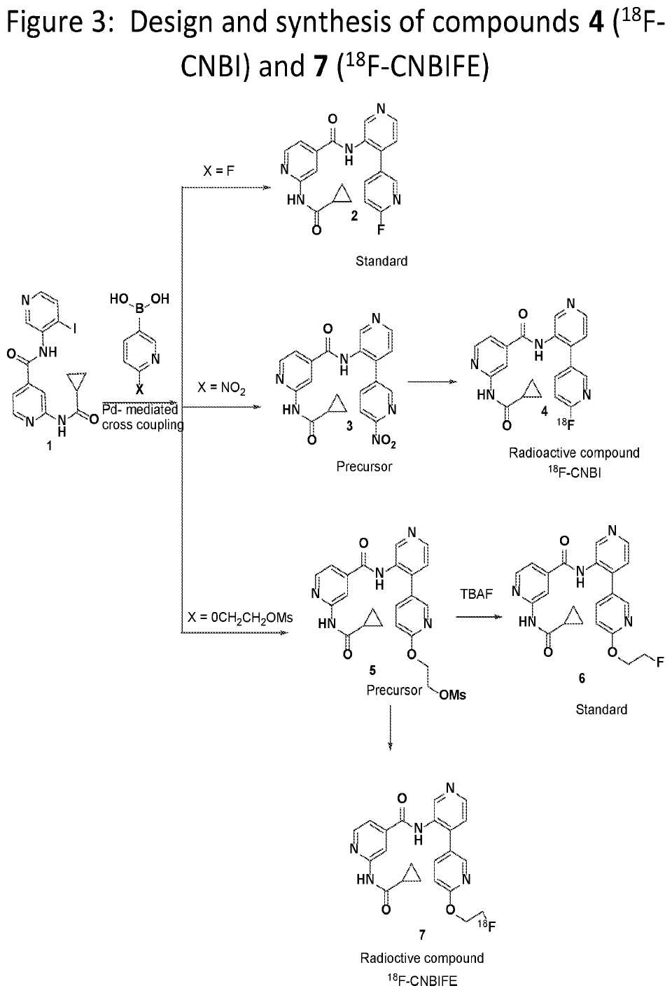

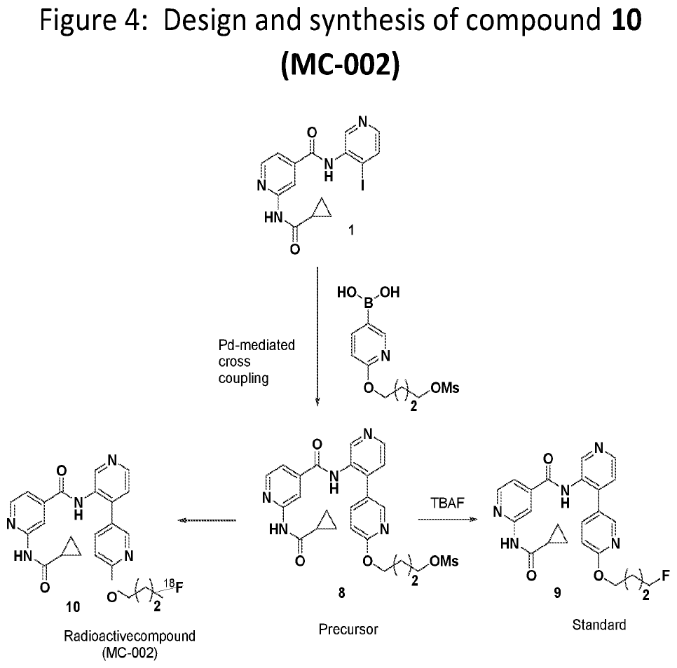

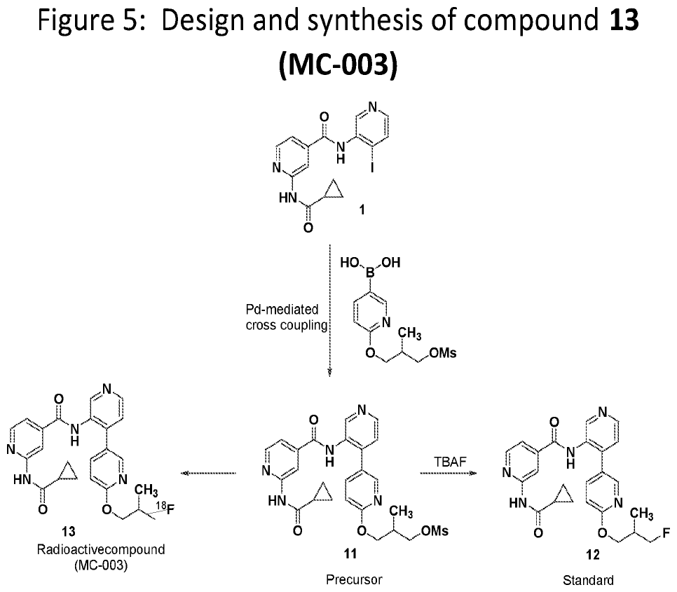

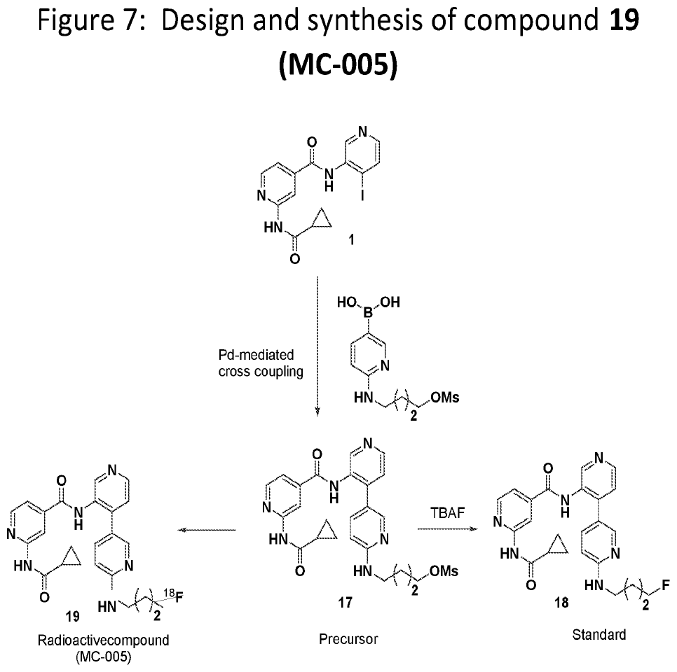

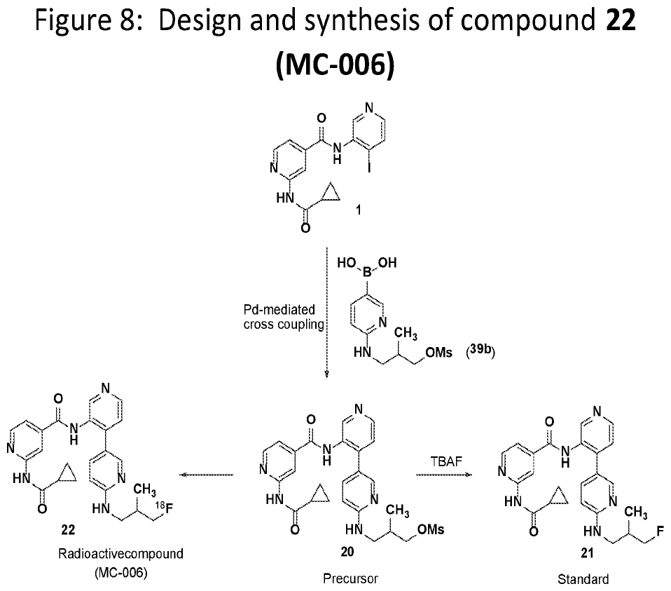

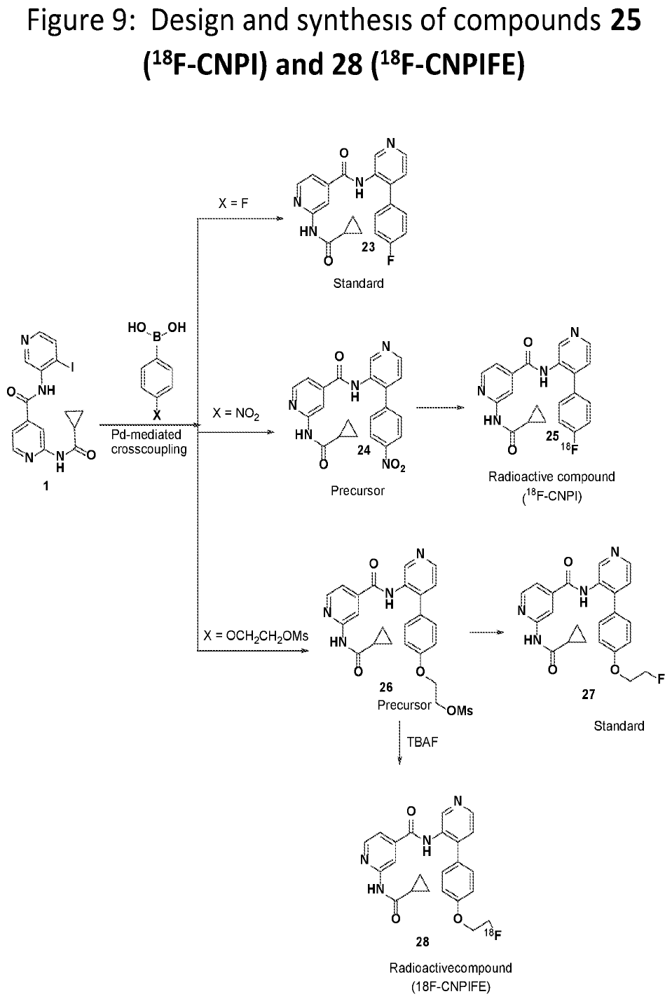

FIGS. 3, 4, 5, 6, 7, and 8 show the Synthetic Routes that can be used for preparing a compound of any of formulas (7), (10), (13), (16), (19), and (22) shown above.

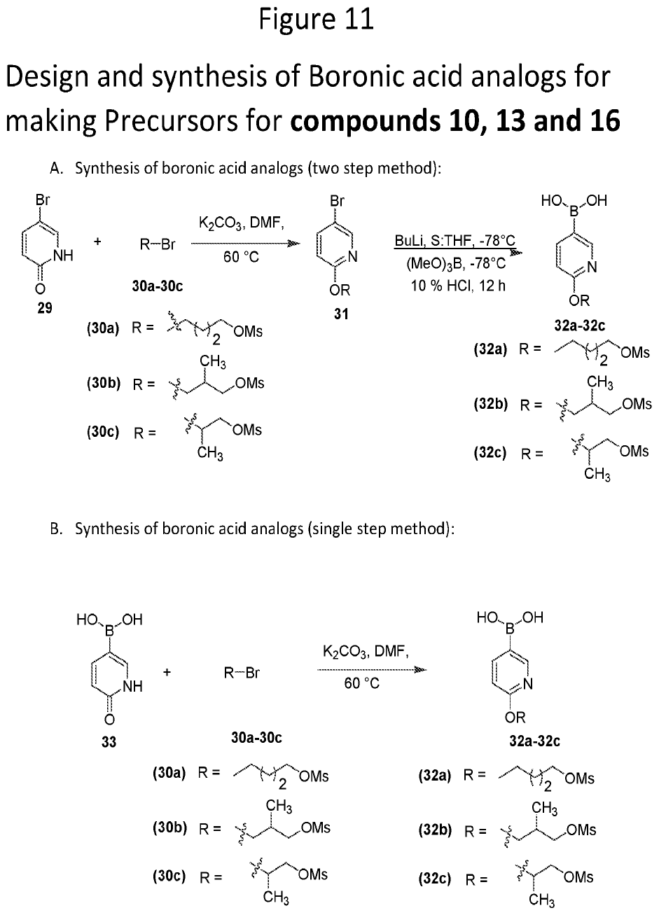

FIG. 11 shows the synthetic routes for boronic acid analogs can be synthesized using two step and single step methods for making precursors for compounds 10, 13 and 16 shown above.

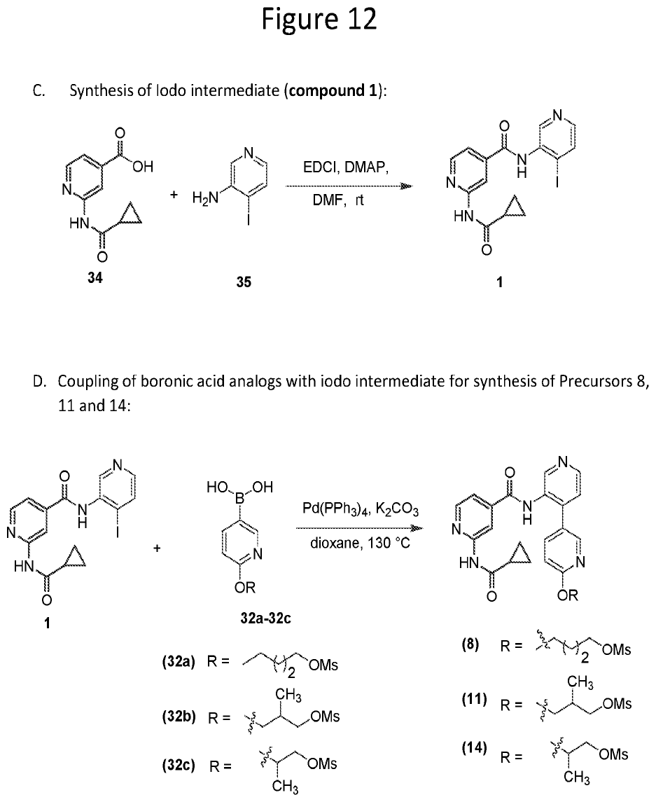

FIG. 12 shows the synthetic route for Iodo intermediate (compound 1) and Coupling of boronic acid analogs with iodo intermediate for synthesis of Precursors 8, 11 and 14 shown above.

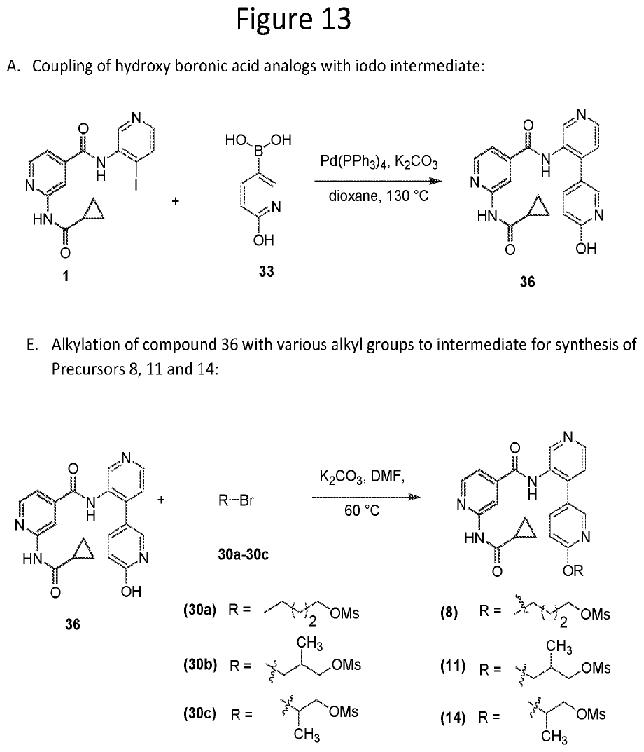

FIG. 13 shows the Coupling of hydroxy boronic acid analogs with iodo intermediate and Alkylation of compound 36 with various alkyl groups which is alternative synthetic method for synthesis of Precursors 8, 11 and 14 shown above.

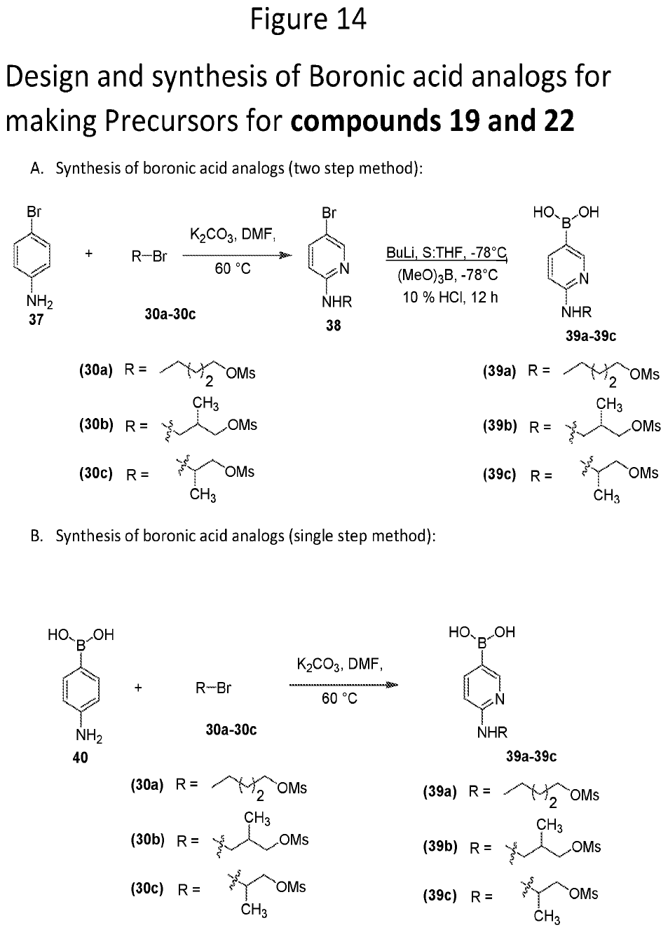

FIG. 14 shows the synthetic routes for boronic acid analogs can be synthesized using two step and single step methods for making precursors for compounds 19 and 22 shown above.

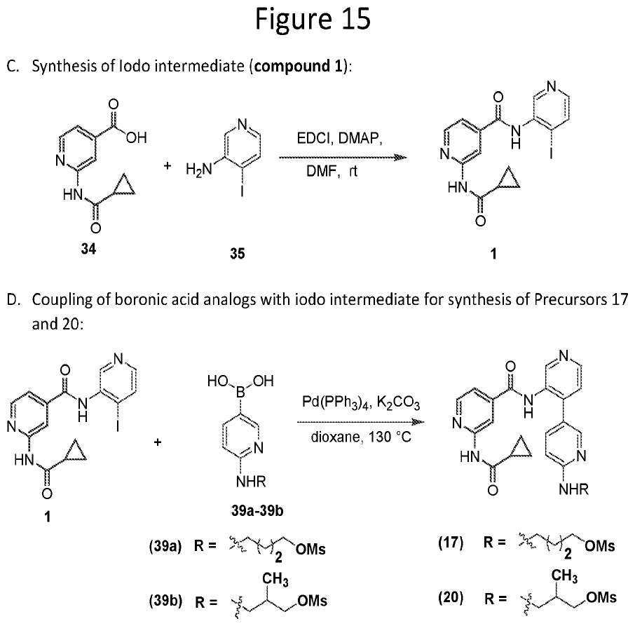

FIG. 15 shows the synthetic route for Iodo intermediate (compound 1) and Coupling of boronic acid analogs with iodo intermediate for synthesis of Precursors 17 and 20 shown above.

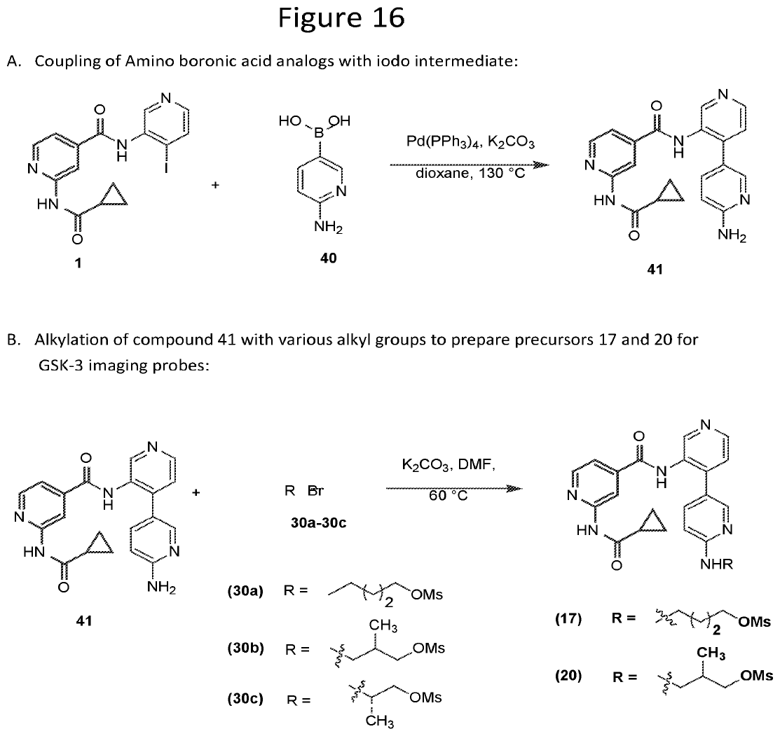

FIG. 16 shows the Coupling of amino boronic acid analogs with iodo intermediate and Alkylation of compound 41 with various alkyl groups which is alternative synthetic method for synthesis of Precursors 17 and 20 shown above.

Radiolabeling with [¹⁸F]F— can be performed on precursors using a standard radio fluorination method using K222(Cryptand), K₂CO₃ and [¹⁸F]F-produced from a cyclotron. In labeled compounds, the fluorine atom can be replaced with a radioactive ¹⁸F.

### Example 3

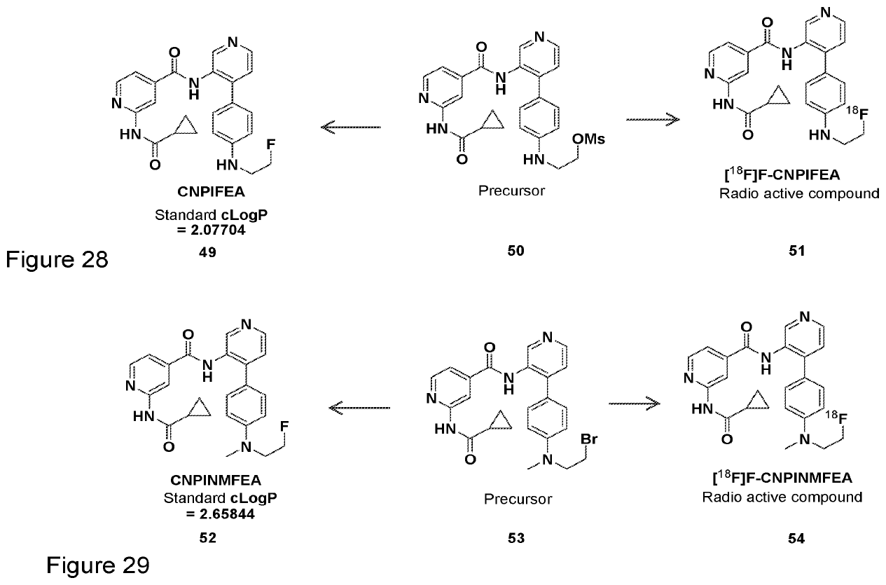

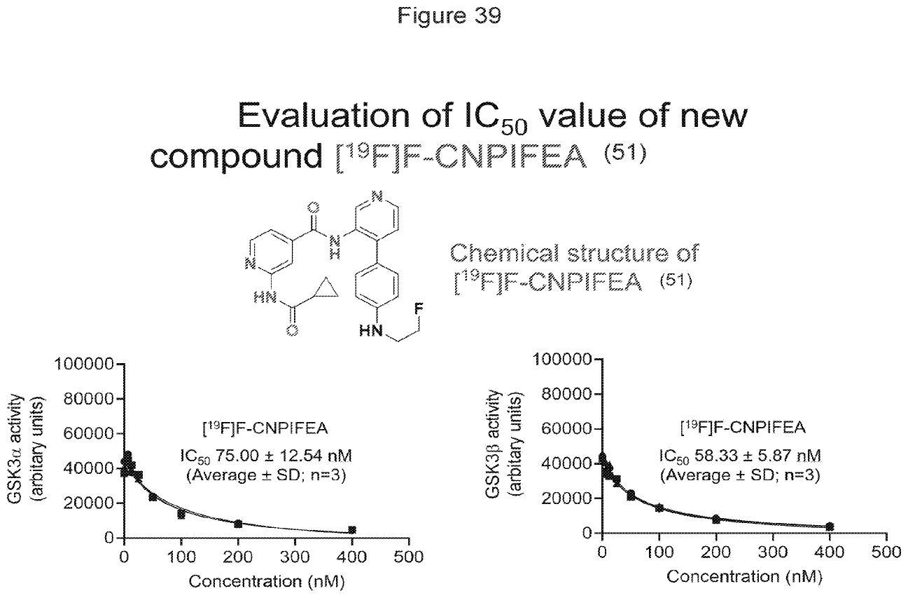

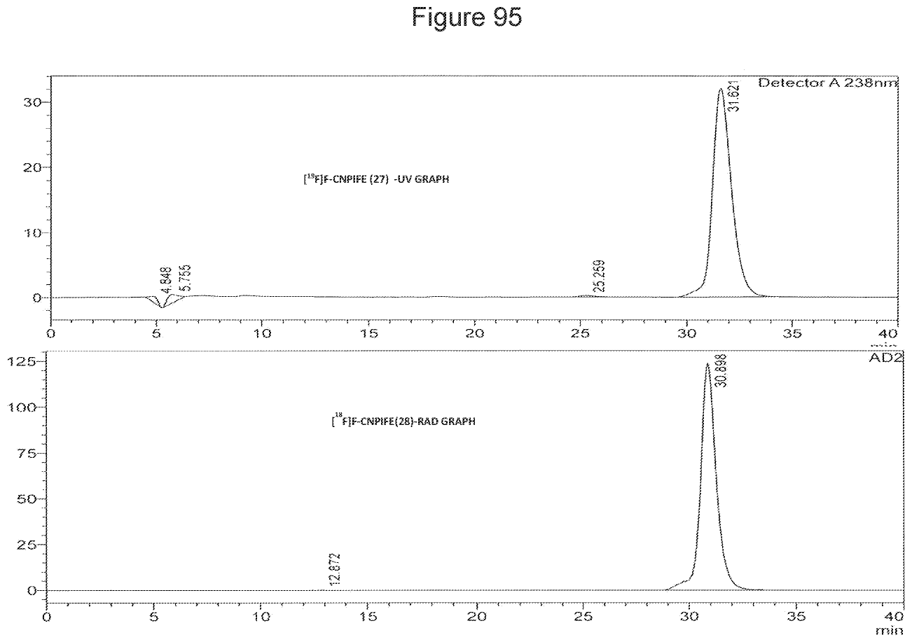

A GSK-3 PET probe [¹⁸F]F-CNPIFEA (51) was synthesized (see FIG. 28). The identity of the synthesized PET probe [¹⁸F]F-CNPIFEA (51) was confirmed (see FIG. 95). FIG. 39 shows a summary of the IC₅₀ value of compound [¹⁸F]F-CNPIFEA (51) measured using an enzyme ADP GIo™ assay as described above.

### Example 4

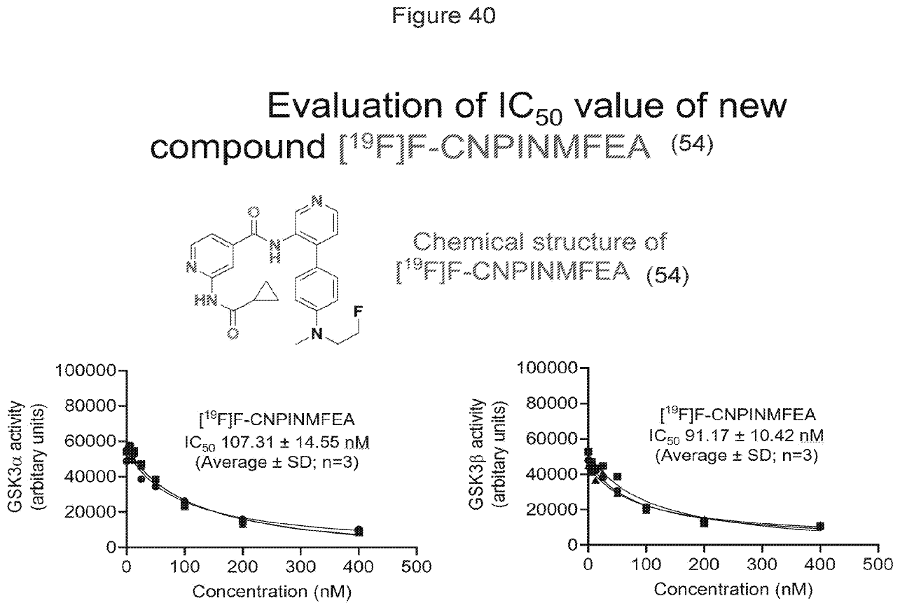

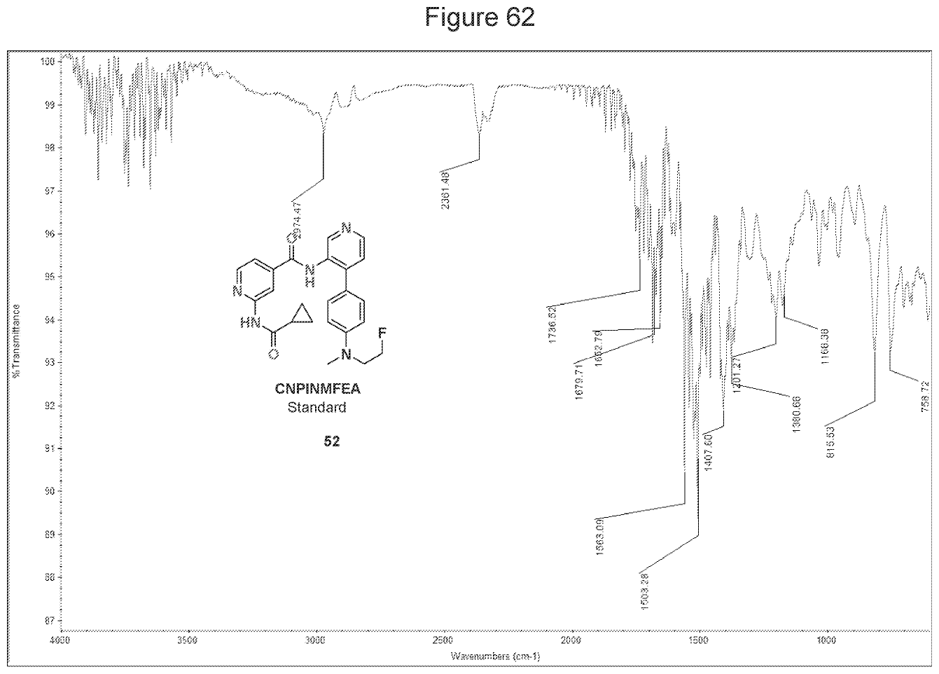

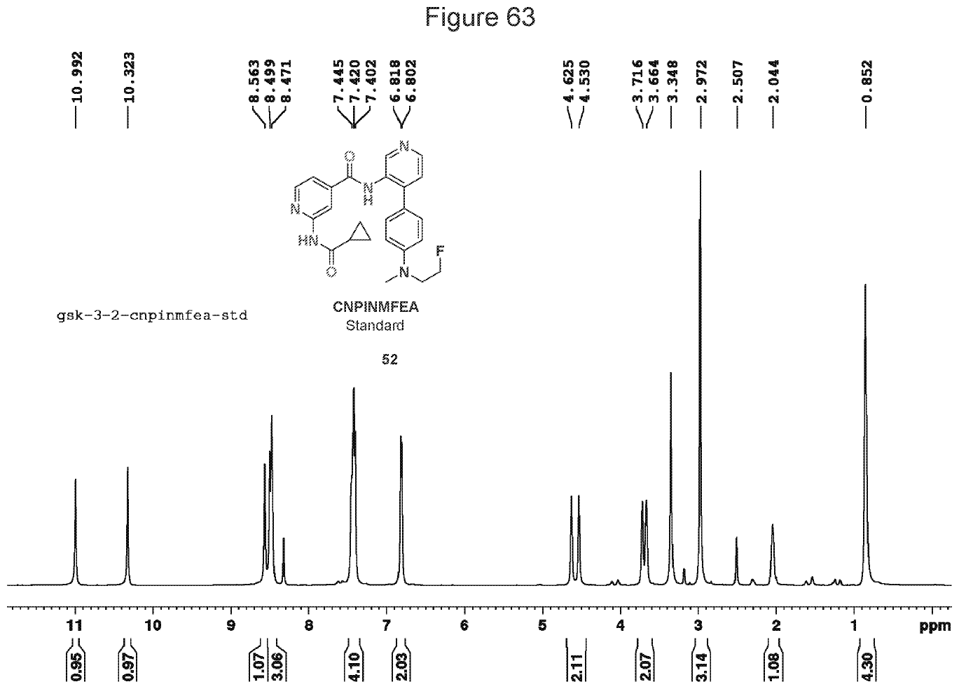

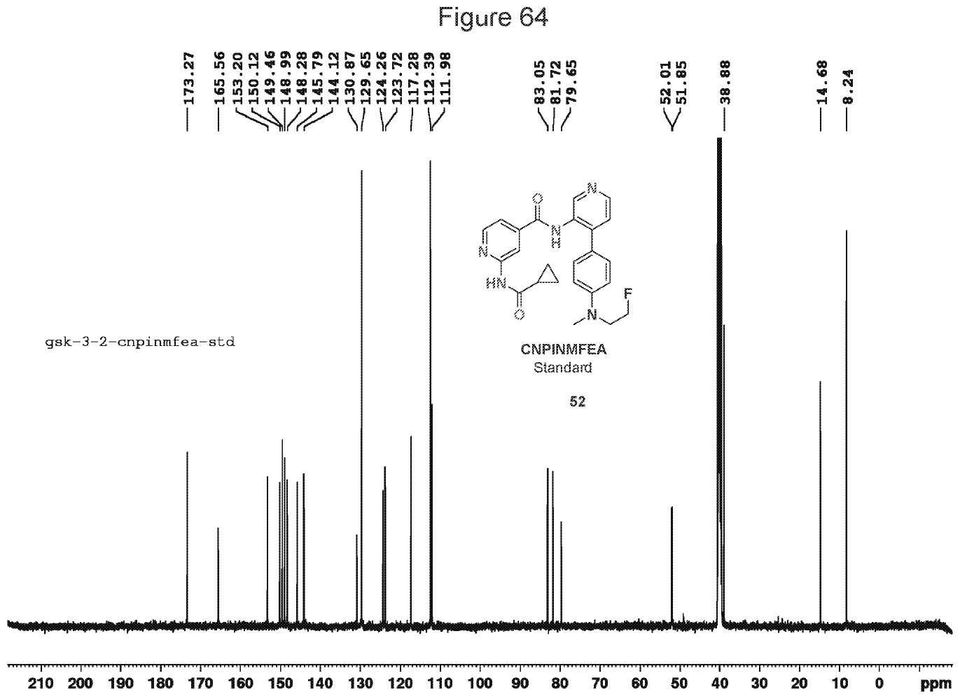

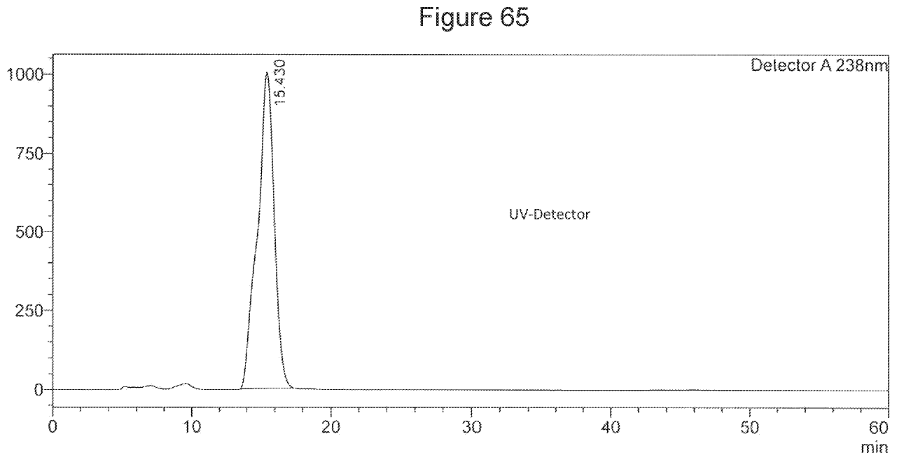

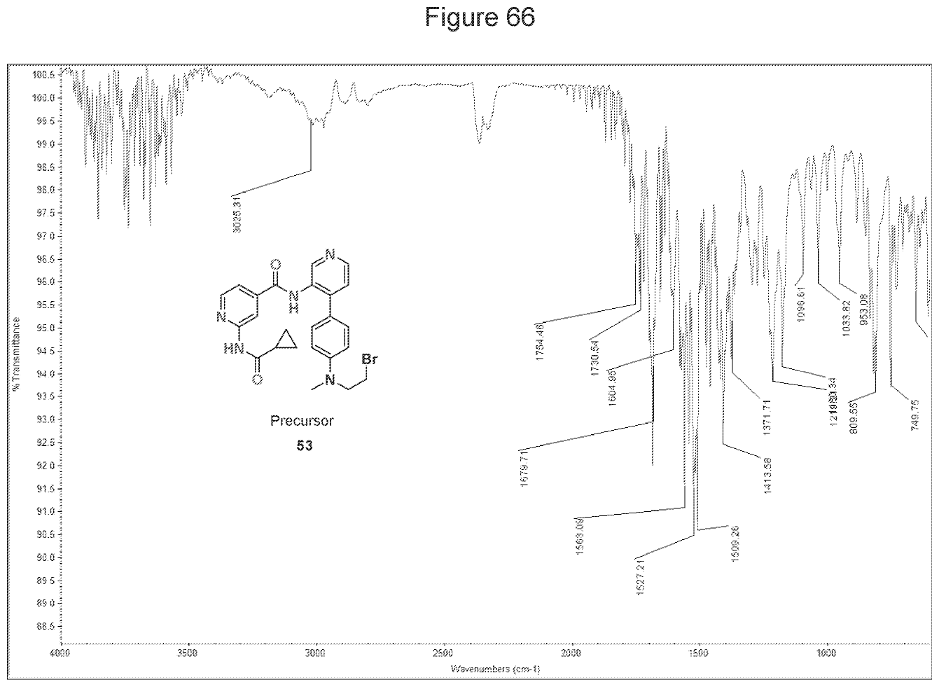

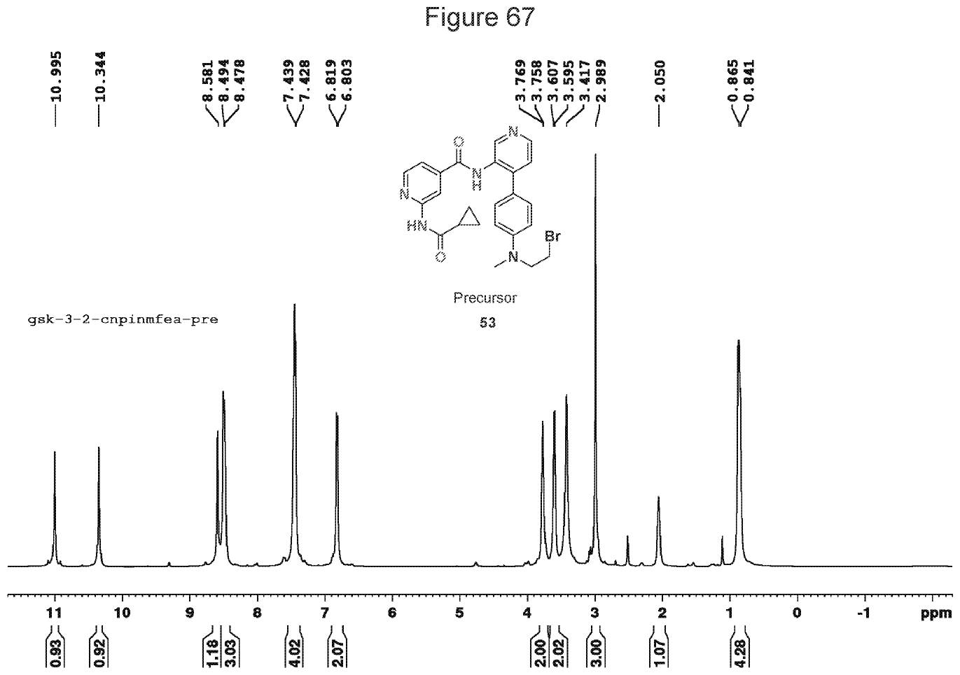

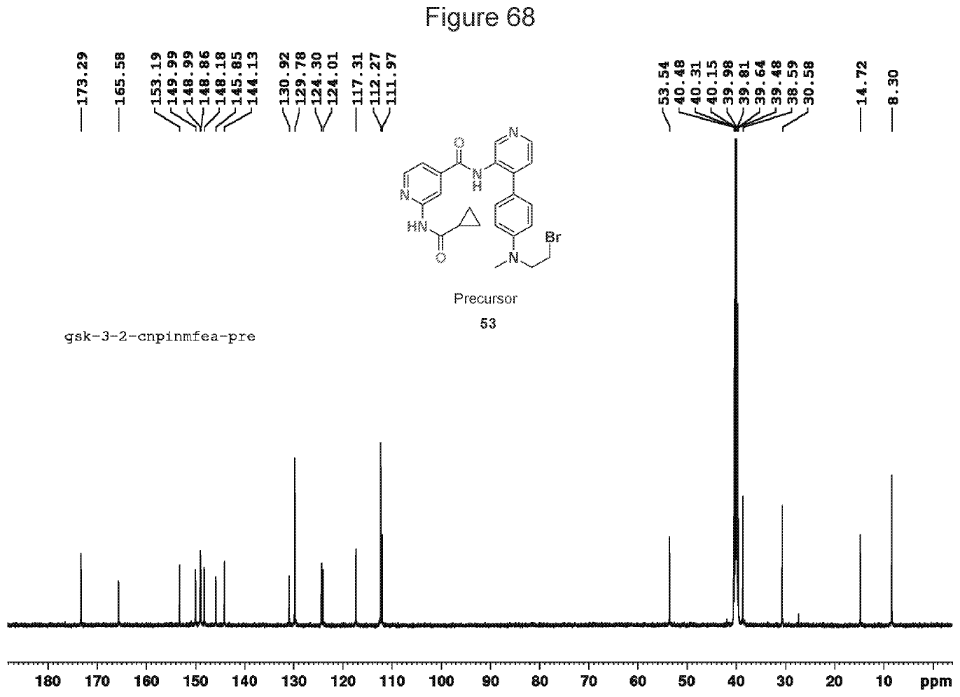

A GSK-3 PET probe [¹⁸F]F-CNPINMFEA (54) was synthesized from a CNPINMFEA standard (52) (see FIG. 29). The identity of the CNPINMFEA standard (52) was confirmed (see FIGS. 62-65). FIG. 40 shows a summary of the IC₅₀ value of compound [¹⁸F]F-CNPINMFEA (54) measured using an enzyme ADP Glo™ assay as described above.

### Example 5

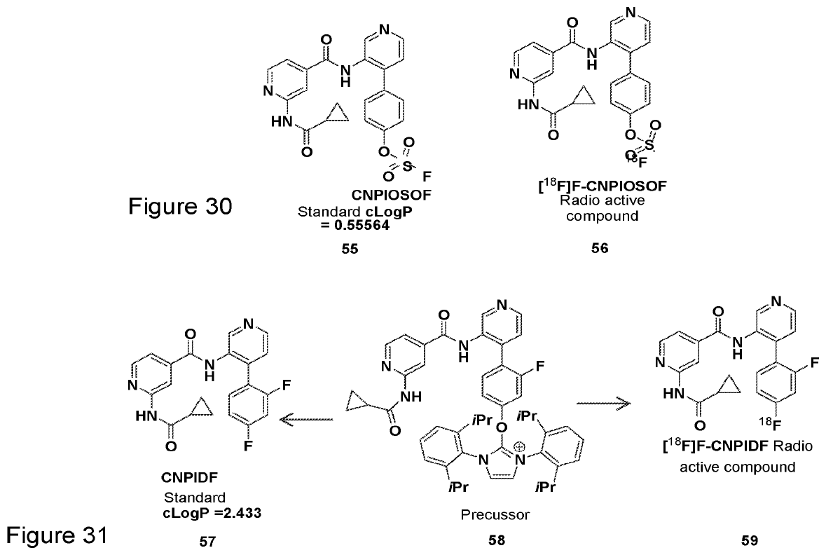

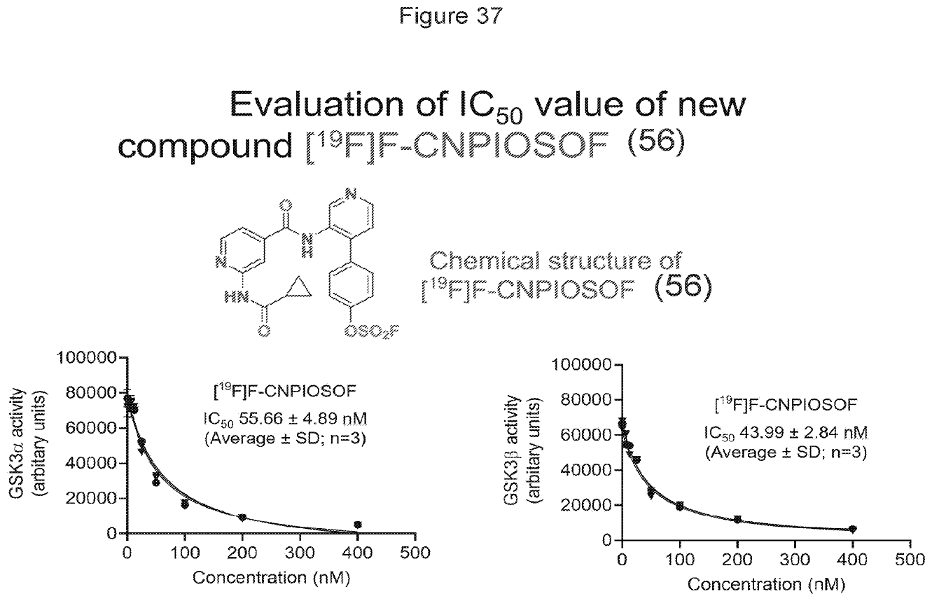

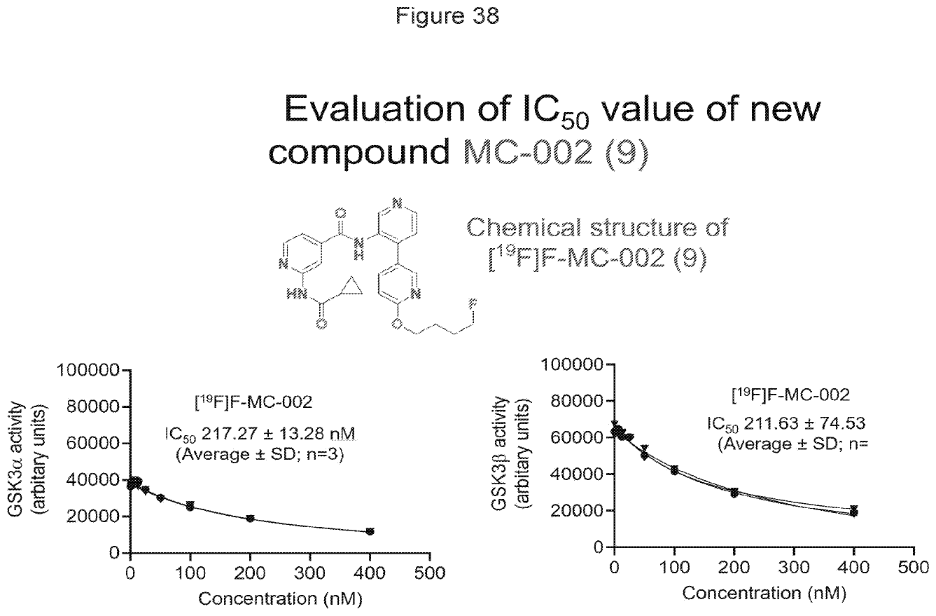

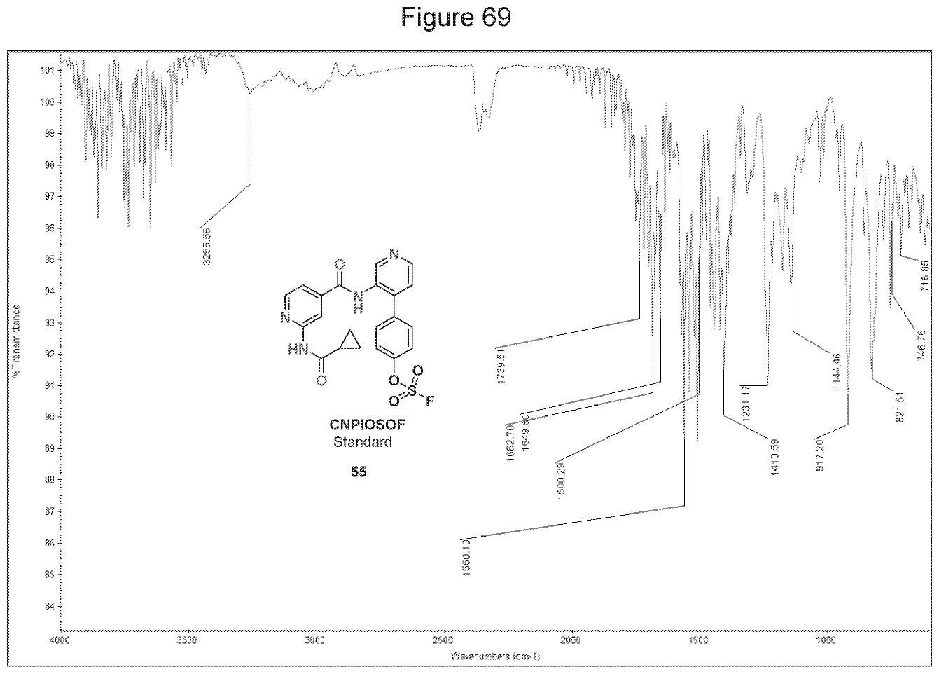

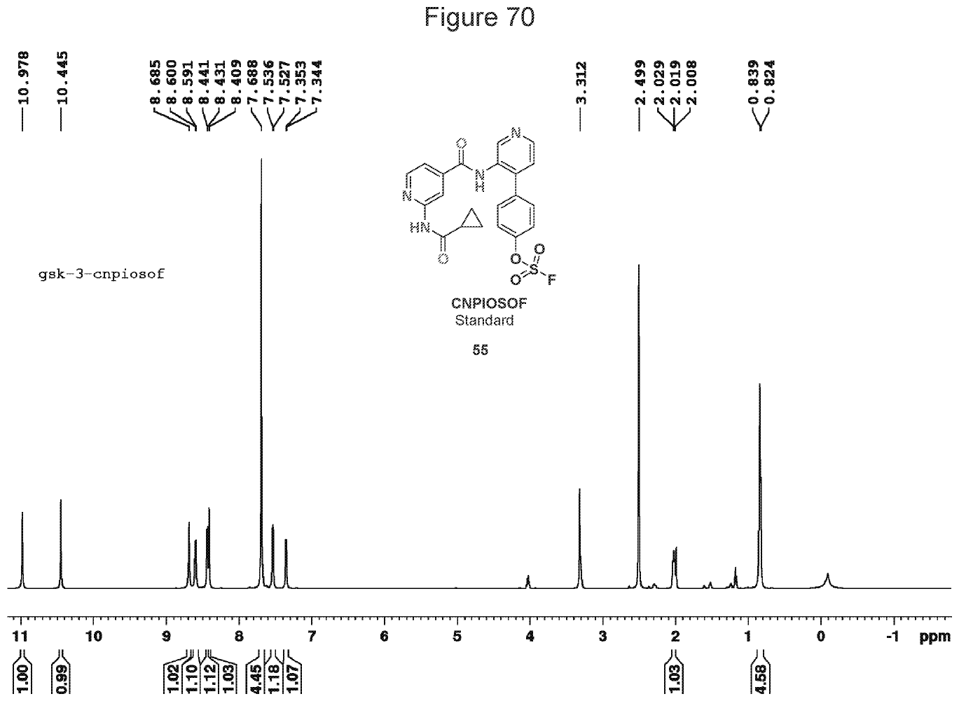

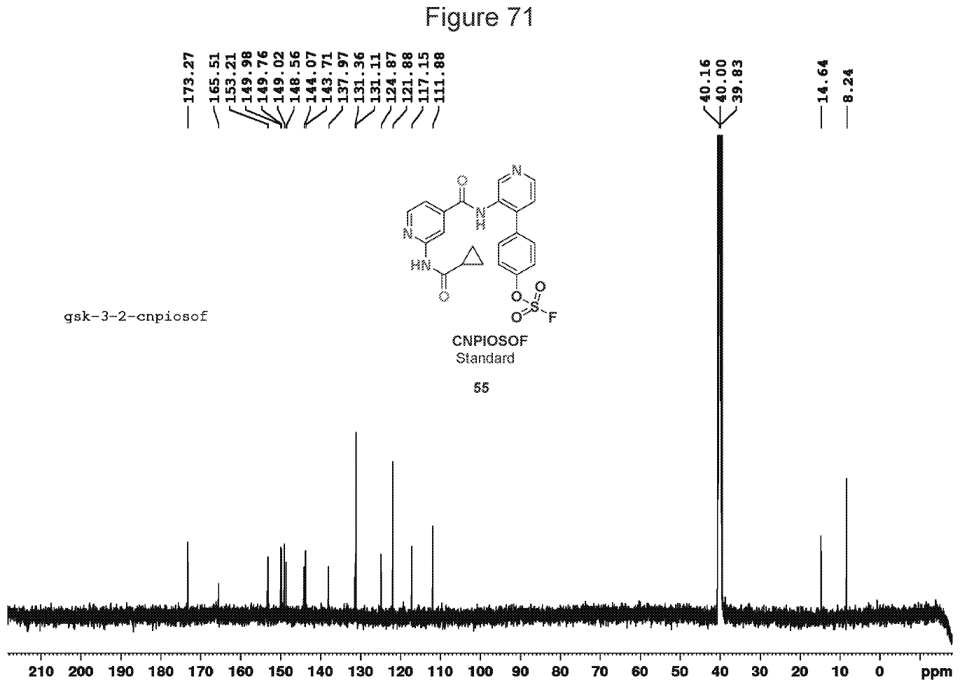

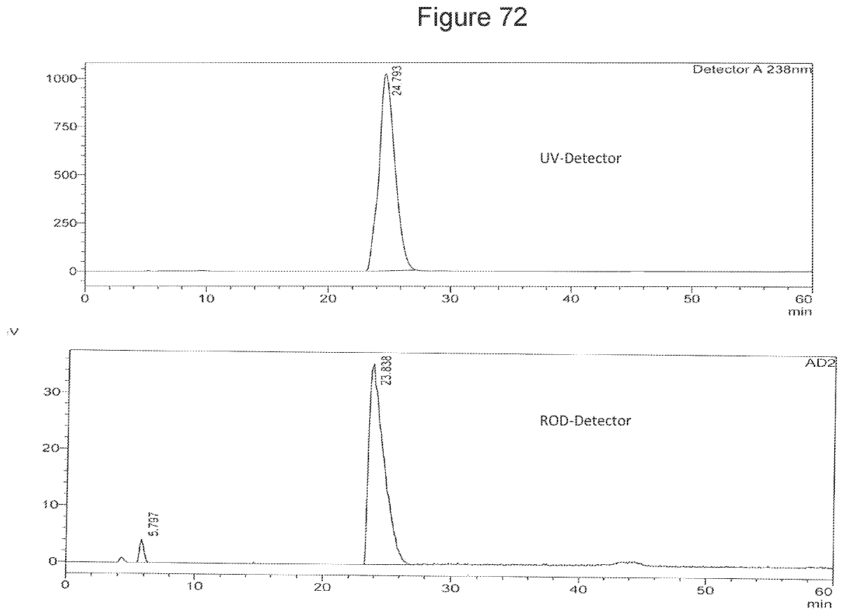

A GSK-3 PET probe [¹⁸F]F—CNPIOSOF (56) was synthesized from a CNPIOSOF standard (55) (see FIG. 30). The identity of the CNPIOSOF standard (55) was confirmed (see FIGS. 69-72). FIG. 37 shows a summary of the IC₅₀ value of compound [¹⁸F]F—CNPIOSOF (56) measured using an enzyme ADP GIo™ assay as described above.

### Example 6

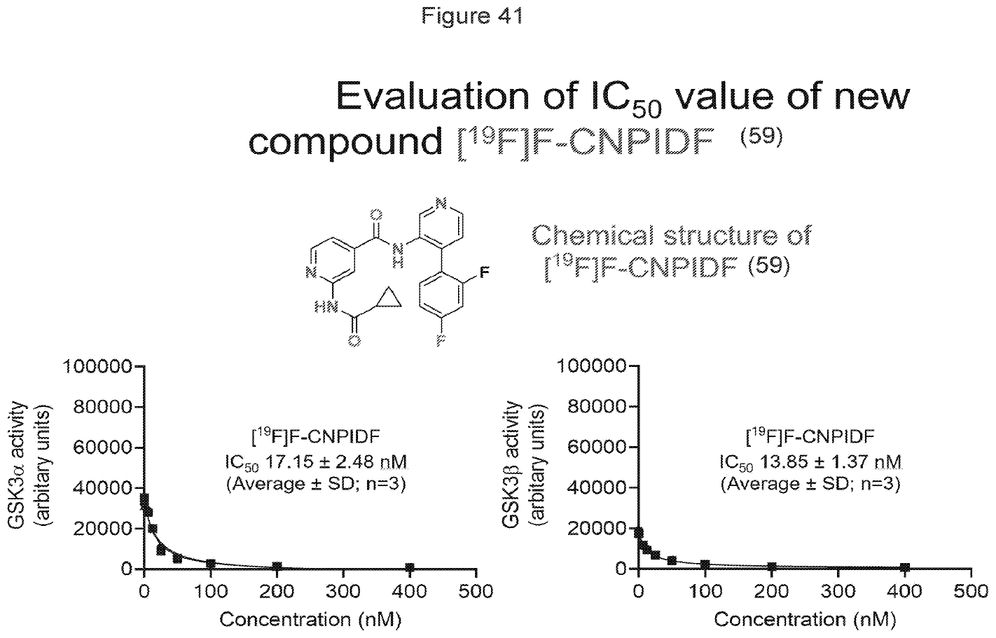

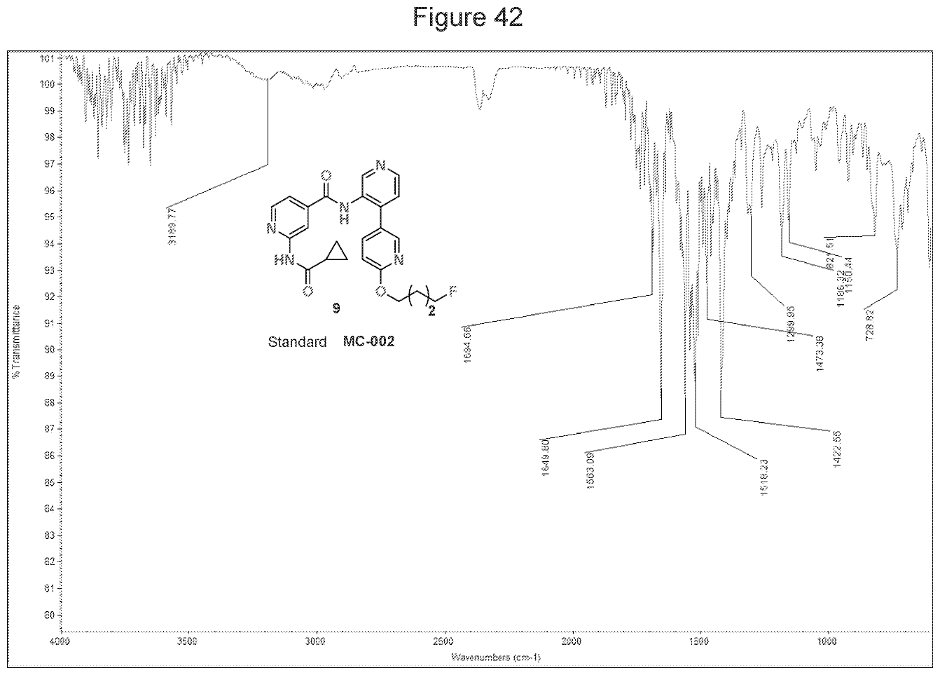

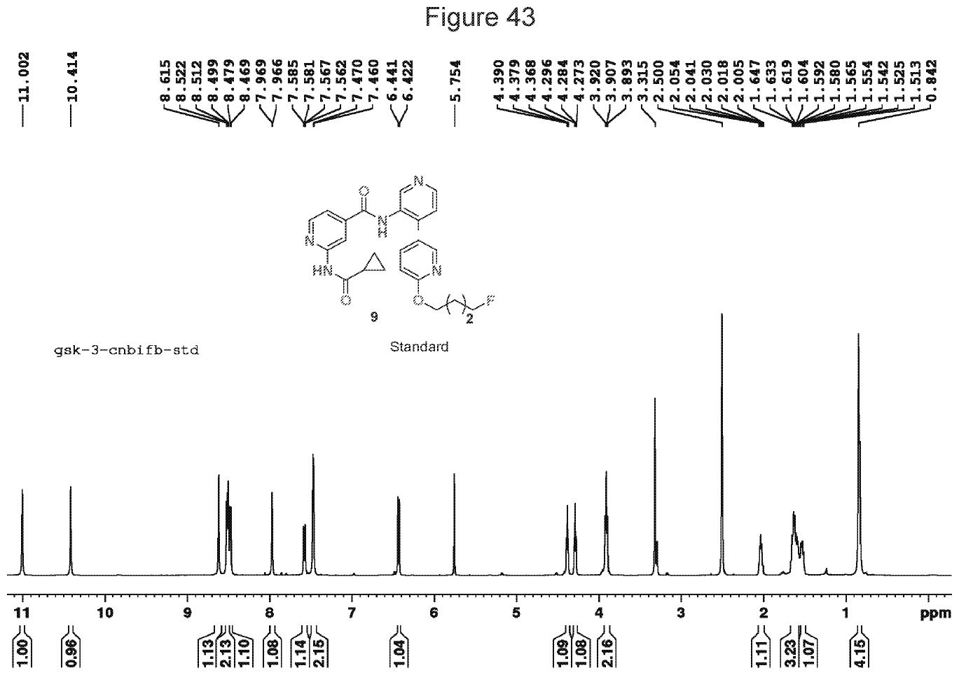

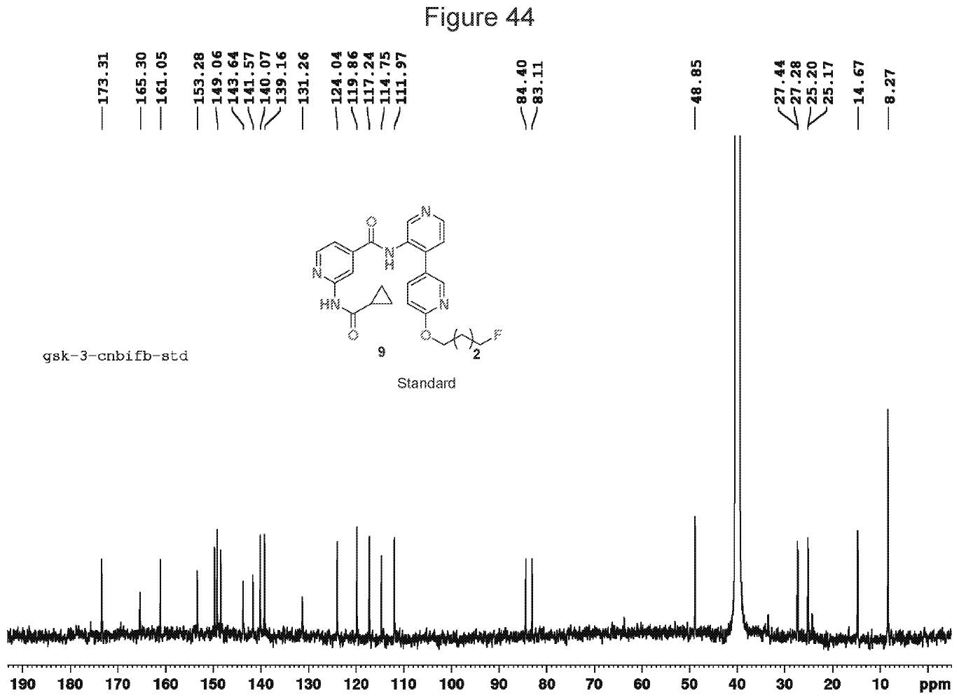

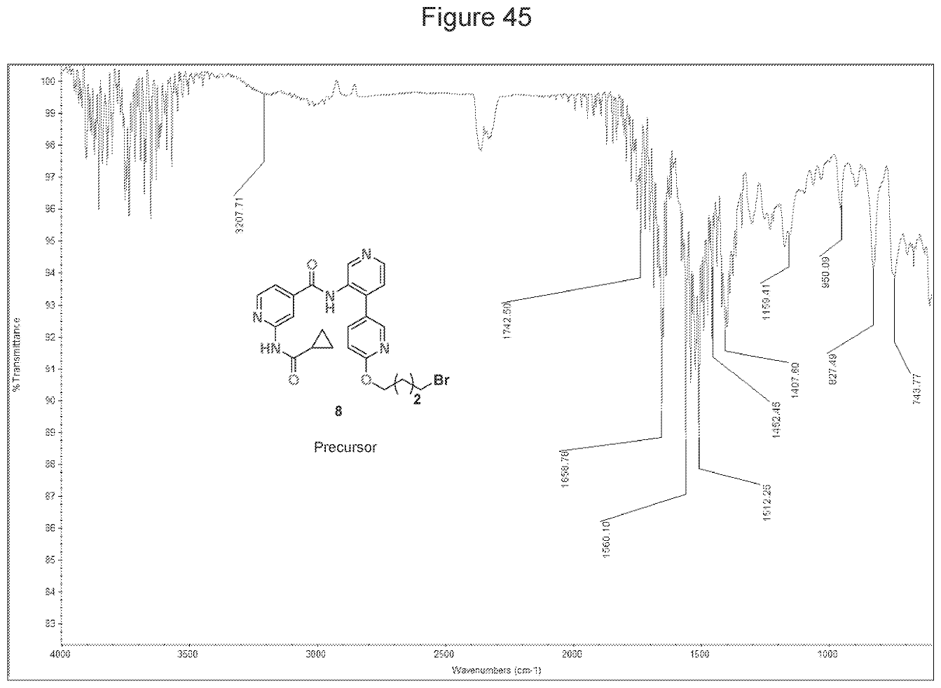

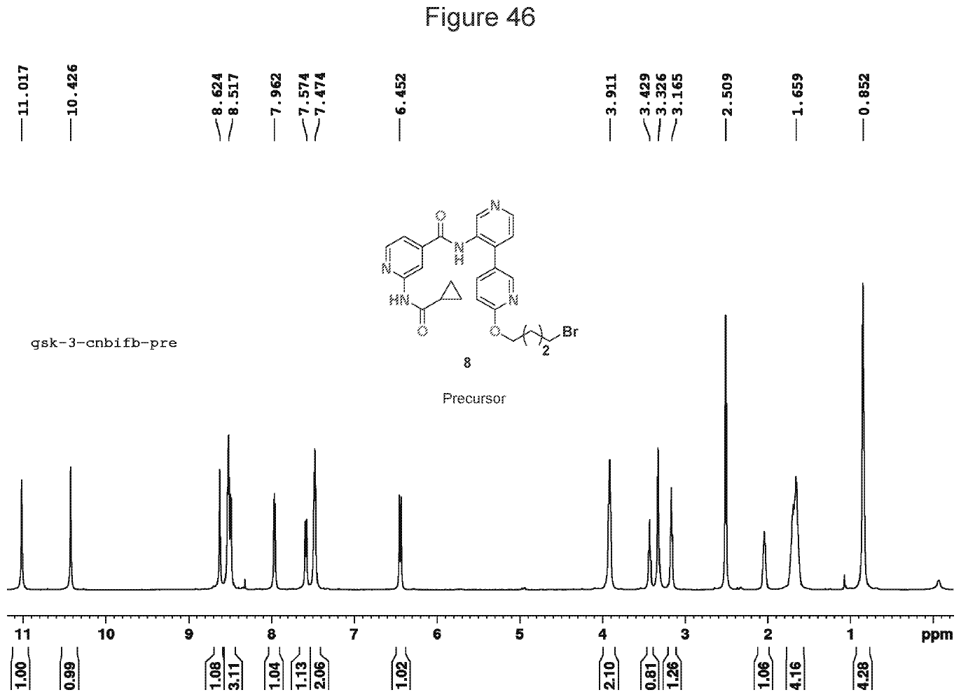

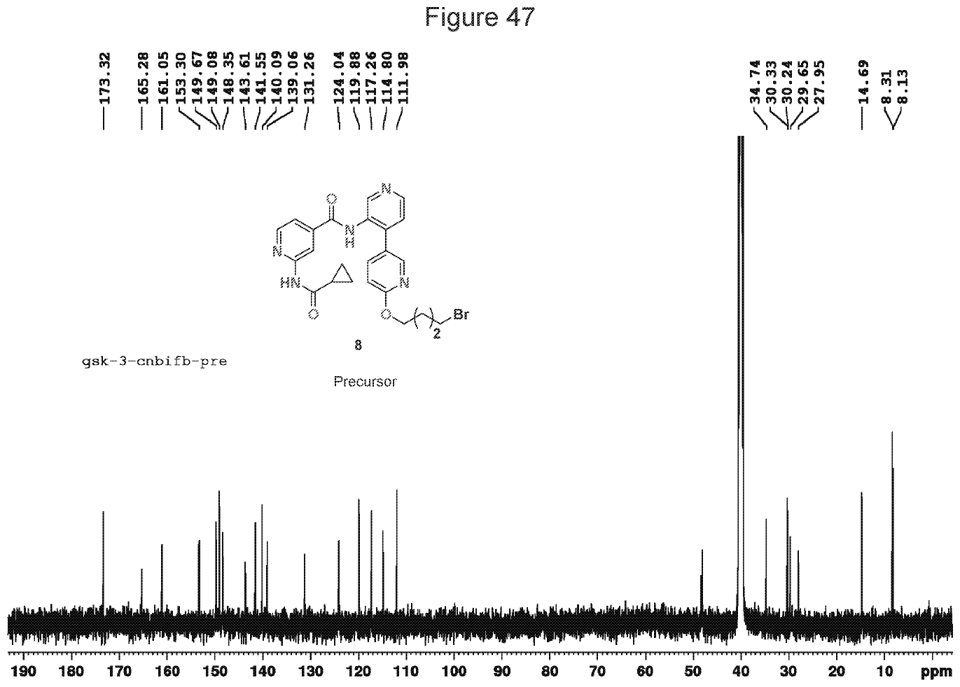

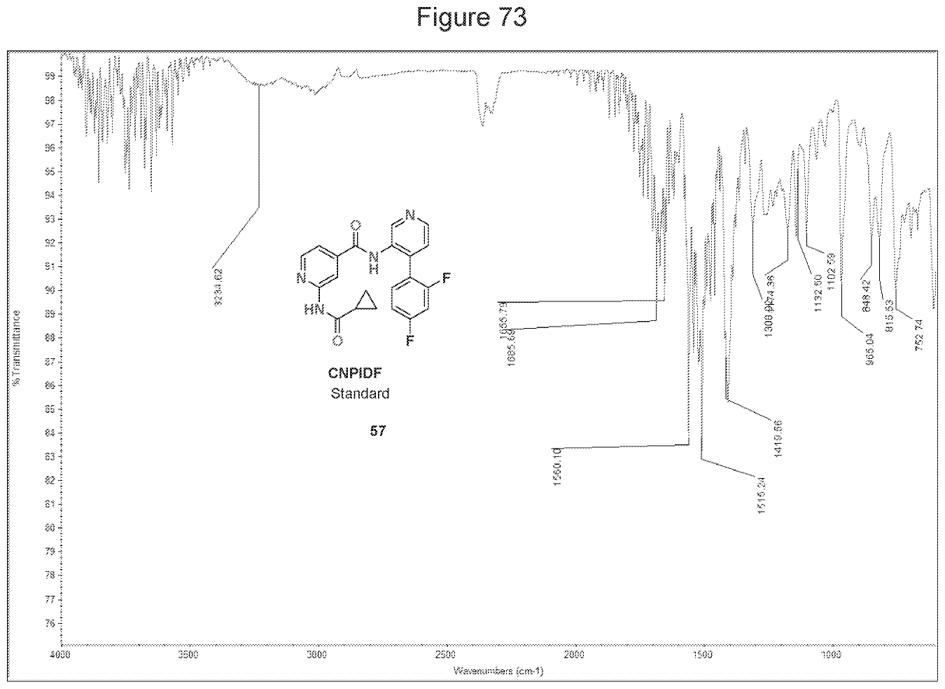

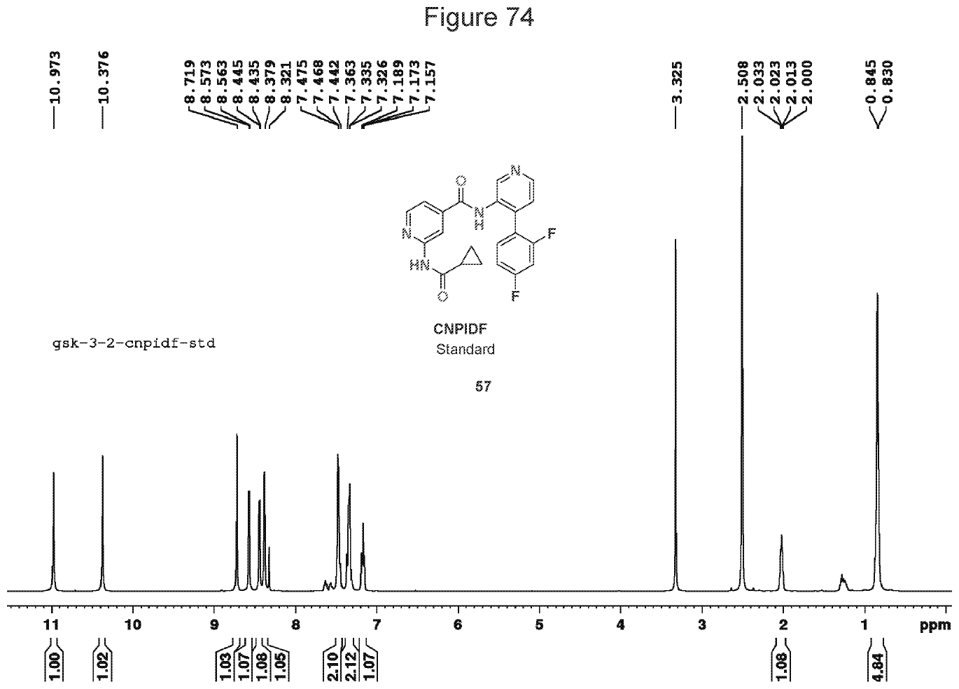

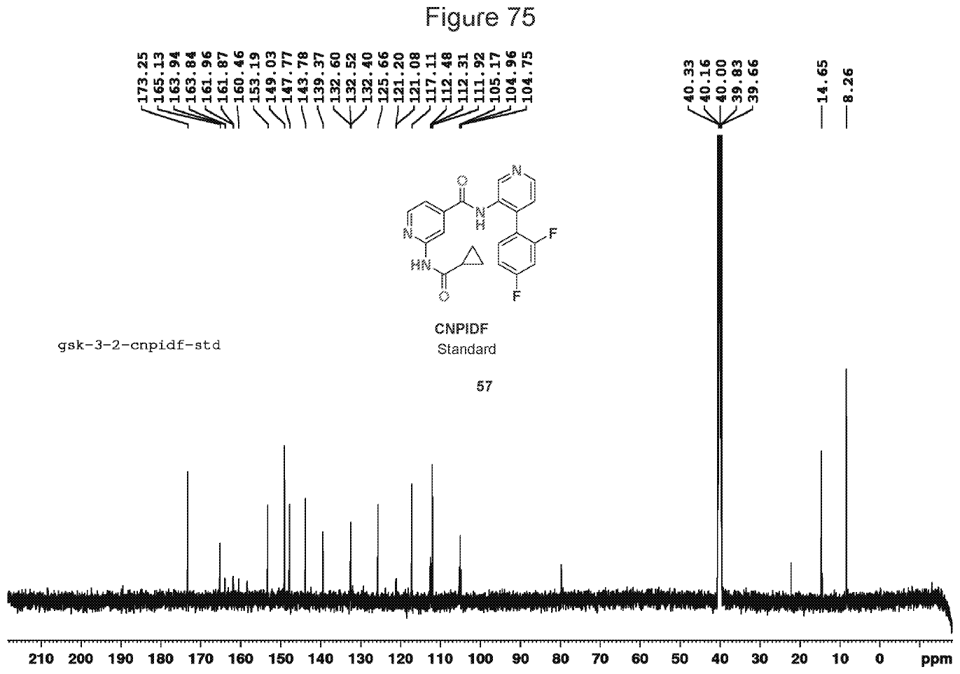

A GSK-3 PET probe [¹⁸F]F-CNPIDF (59) was synthesized from a CNPIDF standard (57) (see FIG. 31). The identity of the CNPIDF standard (57) was confirmed (see FIGS. 73-75). FIG. 41 shows a summary of the IC₅₀ value of compound [¹⁸F]F-CNPIDF (59) measured using an enzyme ADP GIo™ assay as described above.

### Example 7

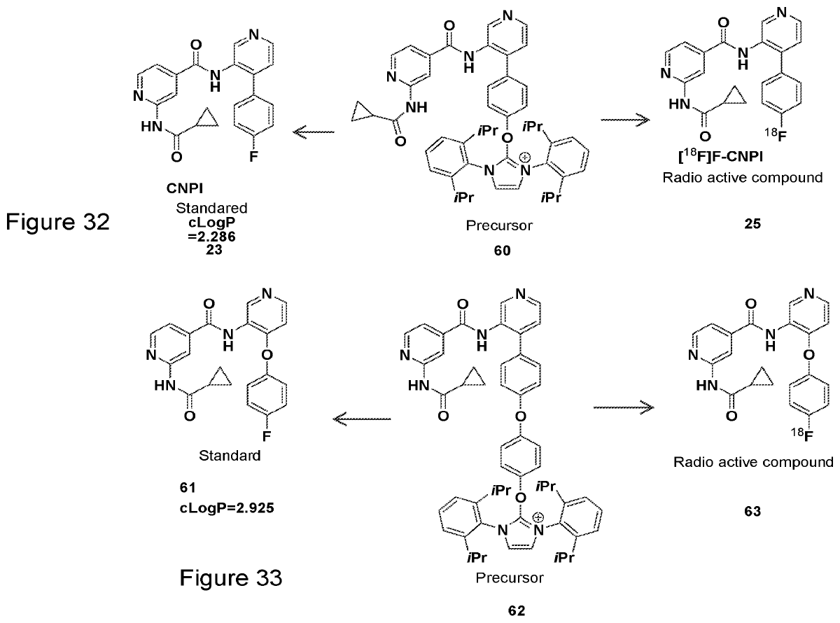

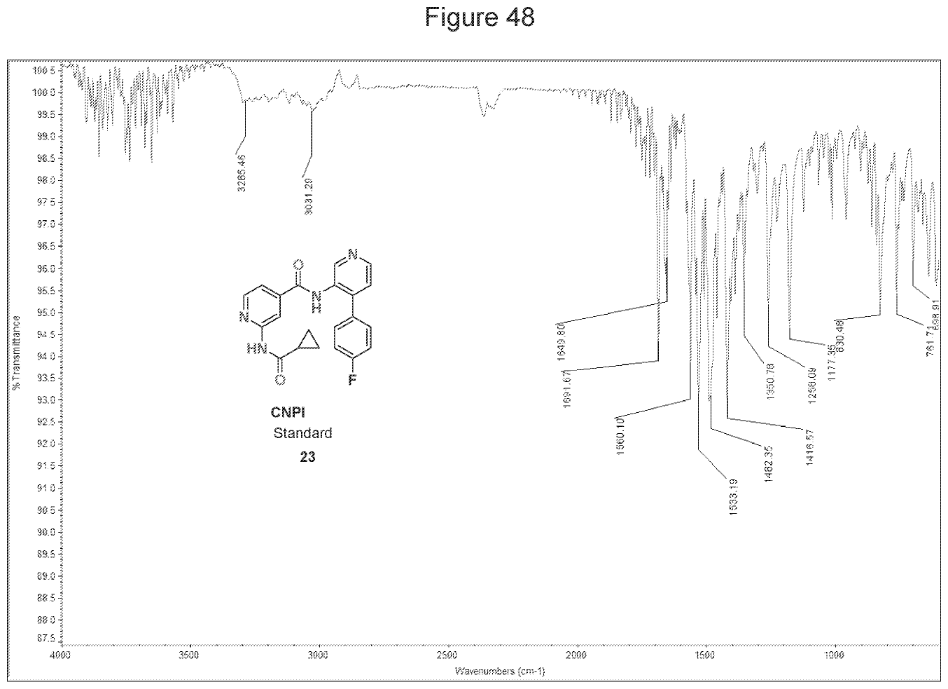

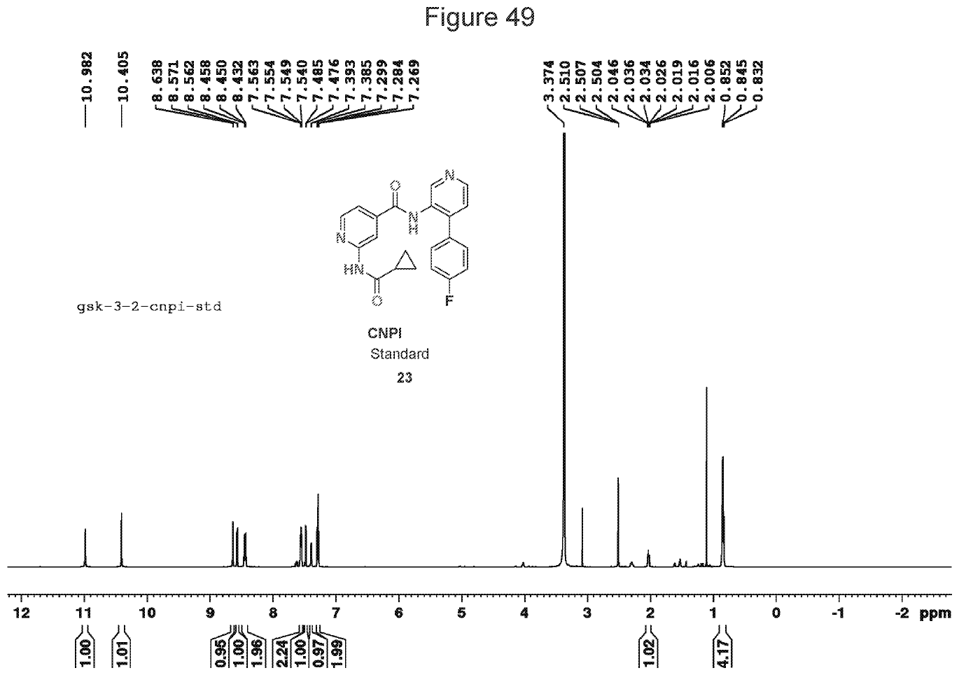

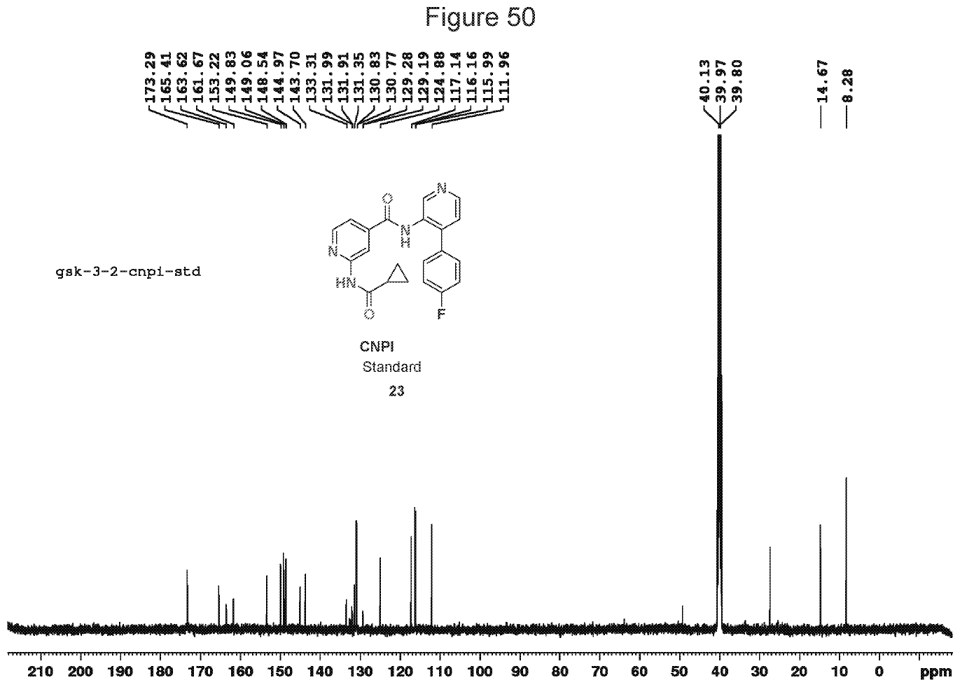

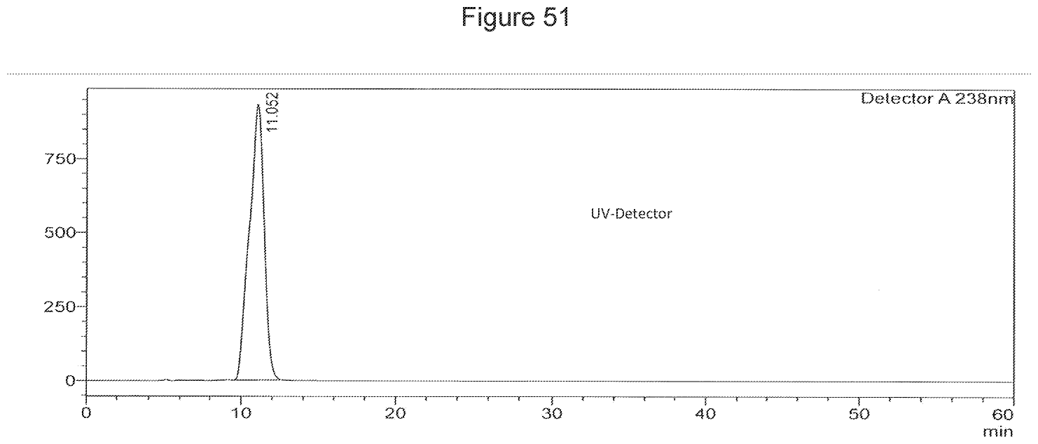

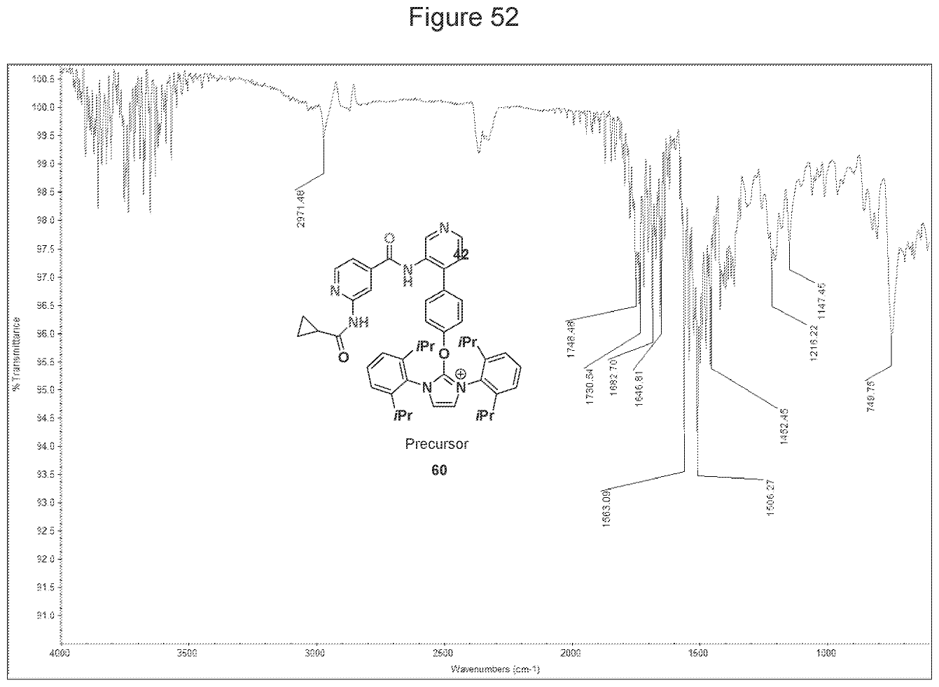

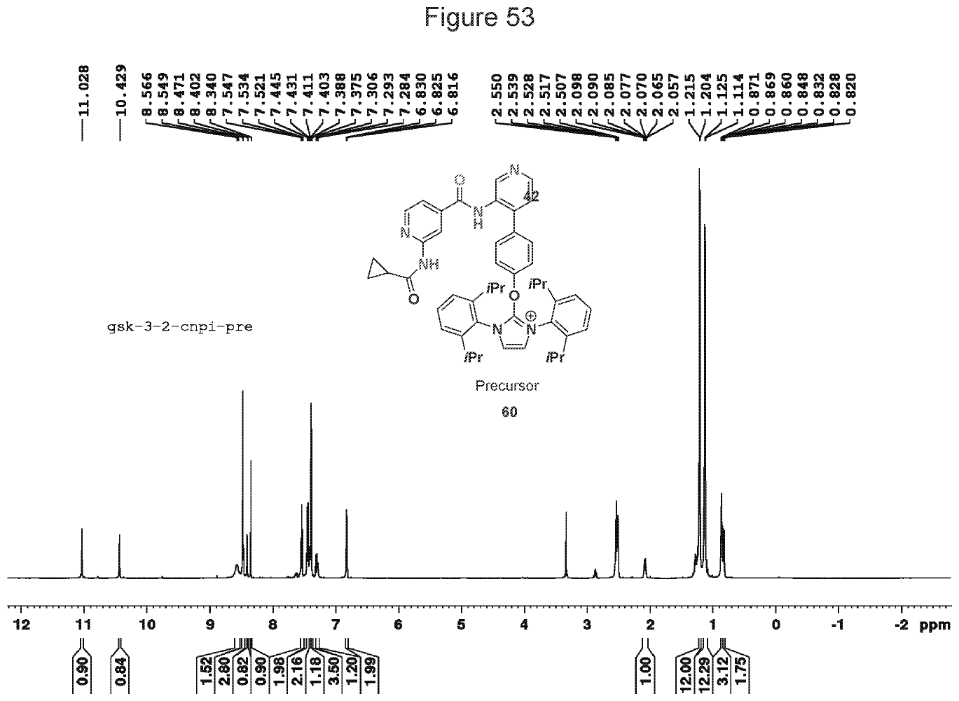

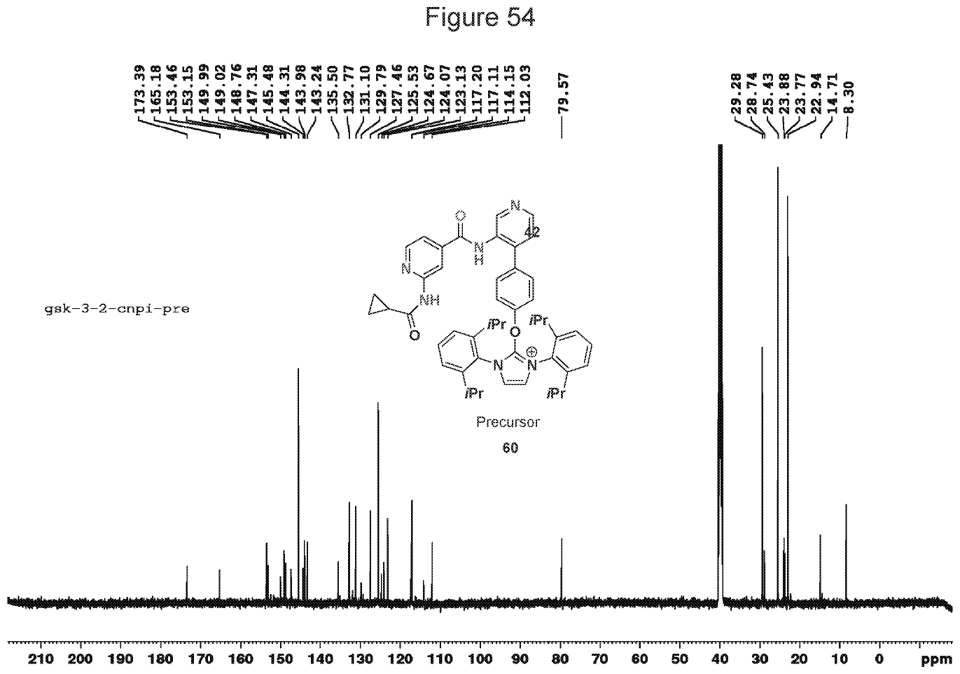

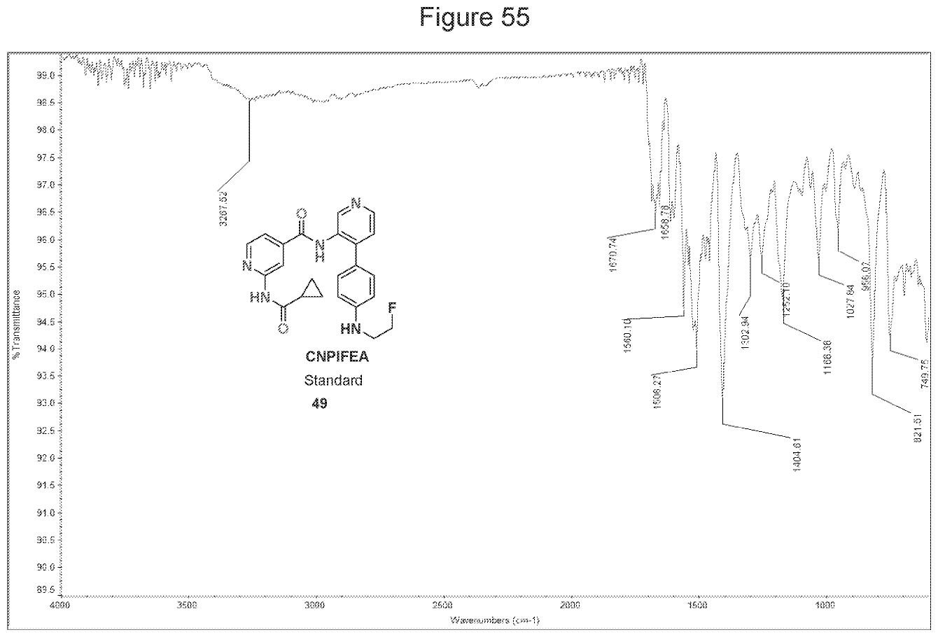

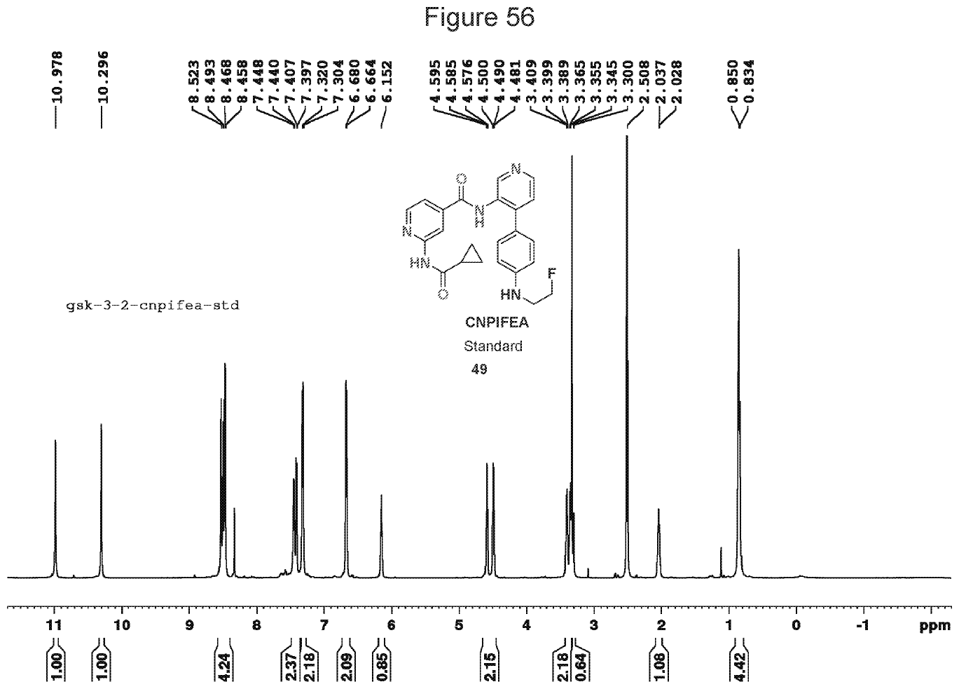

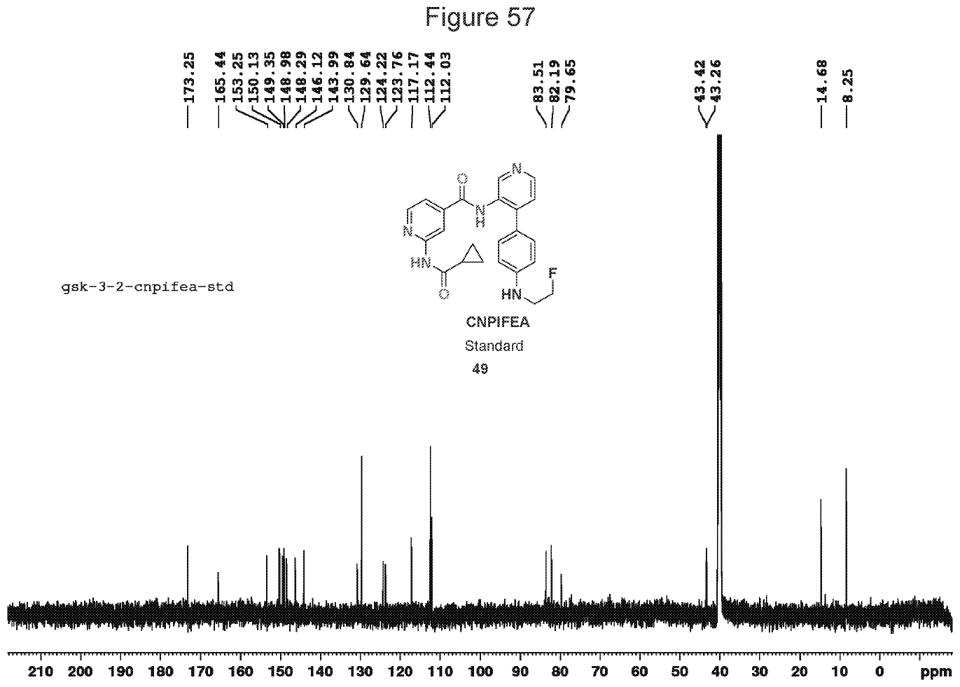

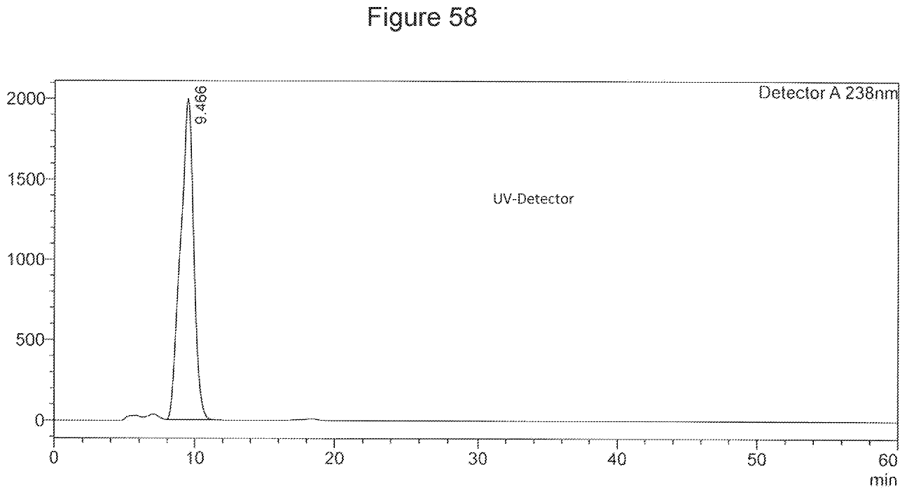

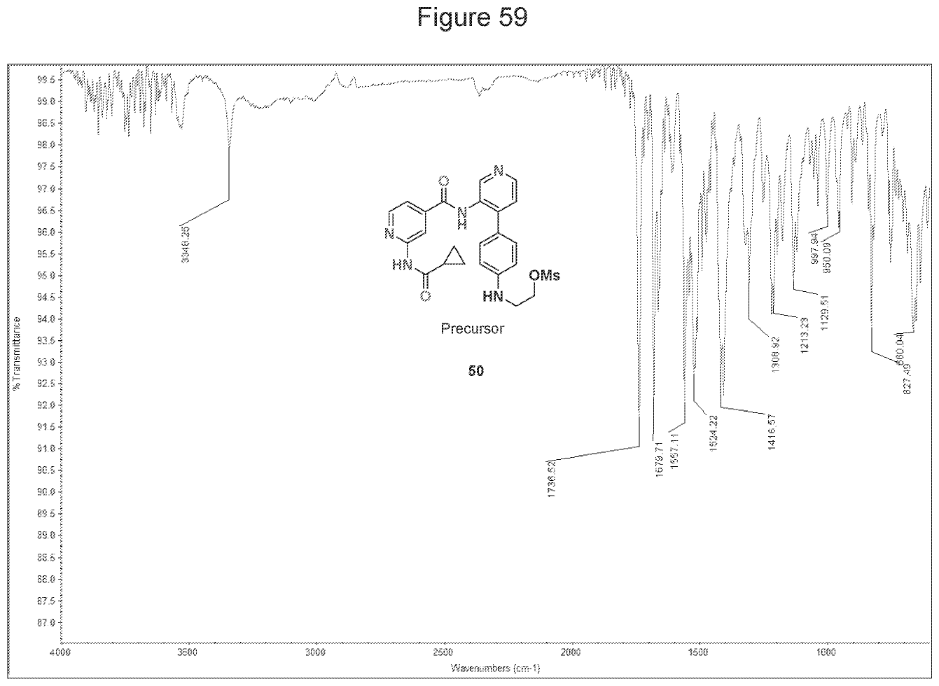

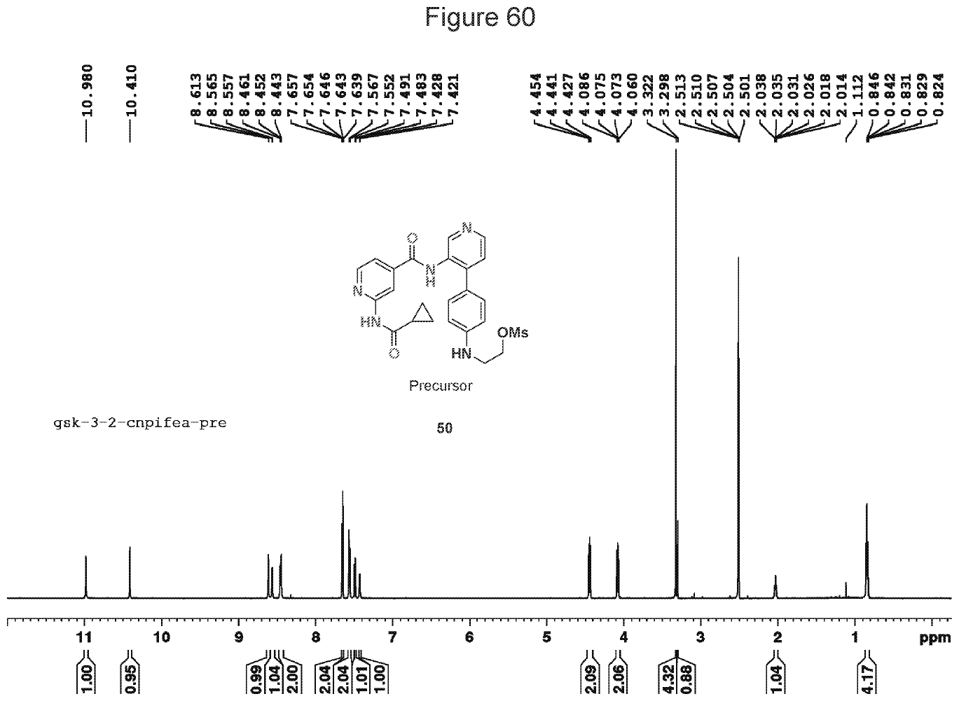

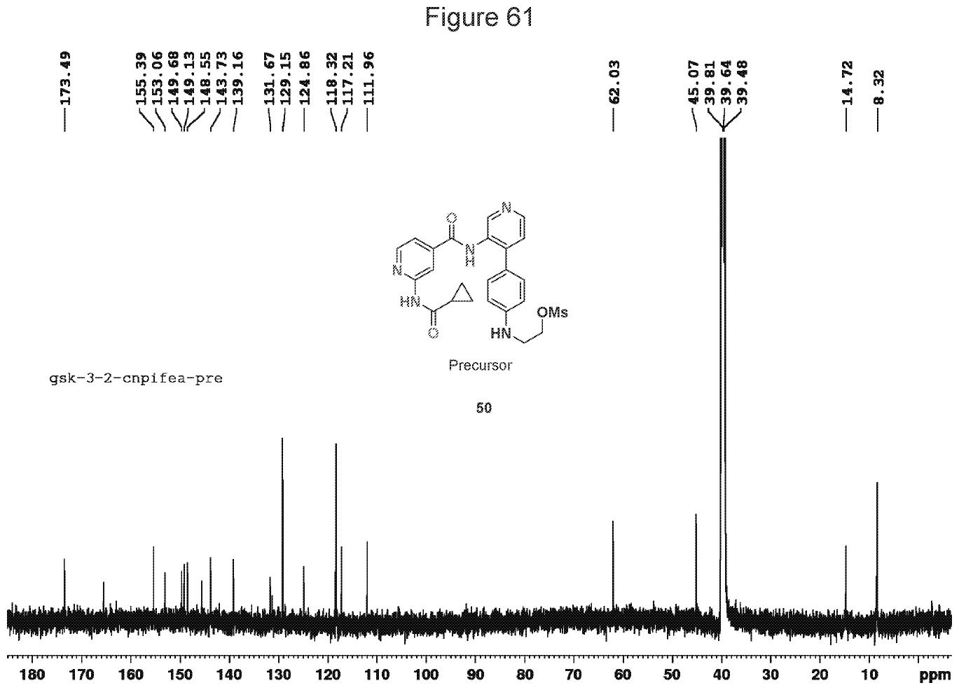

A GSK-3 PET probe [¹⁸F]F—CNPI (25) was synthesized from a CNPI standard (23) (see FIG. 32). The identity of the CNPI standard (23) was confirmed (see FIGS. 48-51).

### Example 8

A GSK-3 PET probe (63) was synthesized (see FIG. 33).

### Example 9

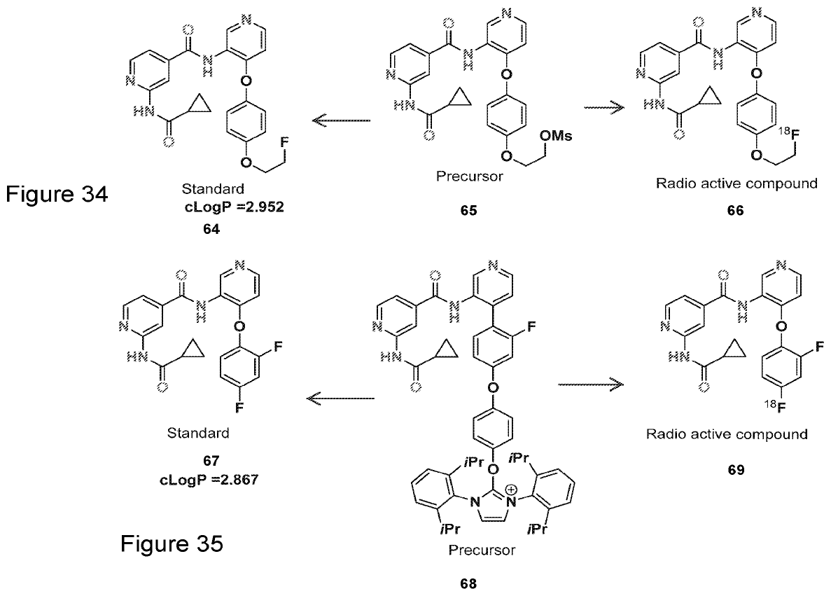

A GSK-3 PET probe (66) was synthesized (see FIG. 34).

### Example 10

A GSK-3 PET probe (69) was synthesized (see FIG. 35).

### Example 11

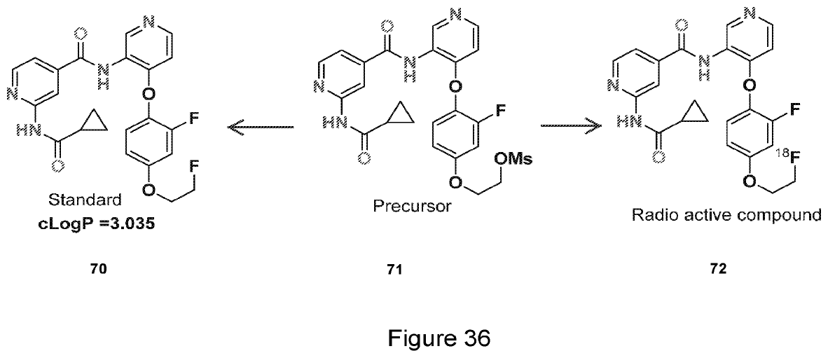

A GSK-3 PET probe (72) was synthesized (see FIG. 36).

### Example 12

### INTRODUCTION

Glycogen synthase kinase-3 (GSK-3) is a serine/threonine kinase with two structurally similar isoforms, GSK-3α and GSK-3β. Phosphorylation of various substrates by GSK-3α/β is responsible for regulation of various complex biological processes, including metabolism, cell integrity, cell signaling, cellular transport, apoptosis, proliferation, and intracellular communication [Refs. 1-5]. As a result, GSK-3α/β is a target for diagnosis and therapy of multiple diseases like cancer, neuroinflammation and neurodegenerative diseases like Alzheimer's disease (AD), Parkinson's disease (PD), and Huntington's disease (HD) [Refs. 6-10].

The role of GSK-3α/β in microtubule destabilization is known, its overall impact on disease progression of AD is still not clear. To better understand the role of GSK-3α/β in the pathophysiology of AD, noninvasive mapping, and assessment of GSK-3α/β levels in brain in the healthy state and during AD progression would be extremely valuable. Specifically, non-invasive imaging with positron emission tomography (PET) can take advantage of high imaging specificity and availability of existing GSK-3α/β inhibitor molecules. Several PET radiotracers targeting GSK-3a/3 have been developed, but most of these PET radiotracers showed low blood-brain barrier (BBB) permeability and or poor GSK-3α/β specificity [Refs. 11-24]. In addition, the majority of the previously developed PET radiotracers were labeled with carbon-11 having a half-life (T_{1/2}) of 20.4 min causing logistical challenges to perform longer studies, including metabolite analysis at later time points.

To better understand the potential application of isonicotinamide-based PET probes, [¹⁸F]F-CNBI and [¹⁸F]F-CNPIFE, in vitro inhibitory activity assays were performed with nonradioactive [¹⁹F]F-CNBI and [¹⁹F]F-CNPIFE versions of the PET probes against both GSK-3α and GSK-3β isoforms. Both the GSK-3α and GSK-3p isoforms of GSK-3 are important in AD pathology. The assessment of BBB permeability of both [¹⁹F]F-CNBI and [¹⁹F]F-CNPIFE were performed using in vitro parallel artificial membrane permeability assay (PAMPA). In addition to the in vitro assays, in vivo biodistribution studies were also performed for both [¹⁸F]F-CNBI and [¹⁸F]F-CNPIFE PET probes with and without self-blocking with corresponding non-radiolabeled reference compounds in normal FVB/NJ mice. The FVB/NJ mouse model was chosen as opposed to a rat model because it is extensively used as a control for well-established preclinical mouse models for various neuropathies including AD [Refs. 25-27].

### Chemical Synthesis

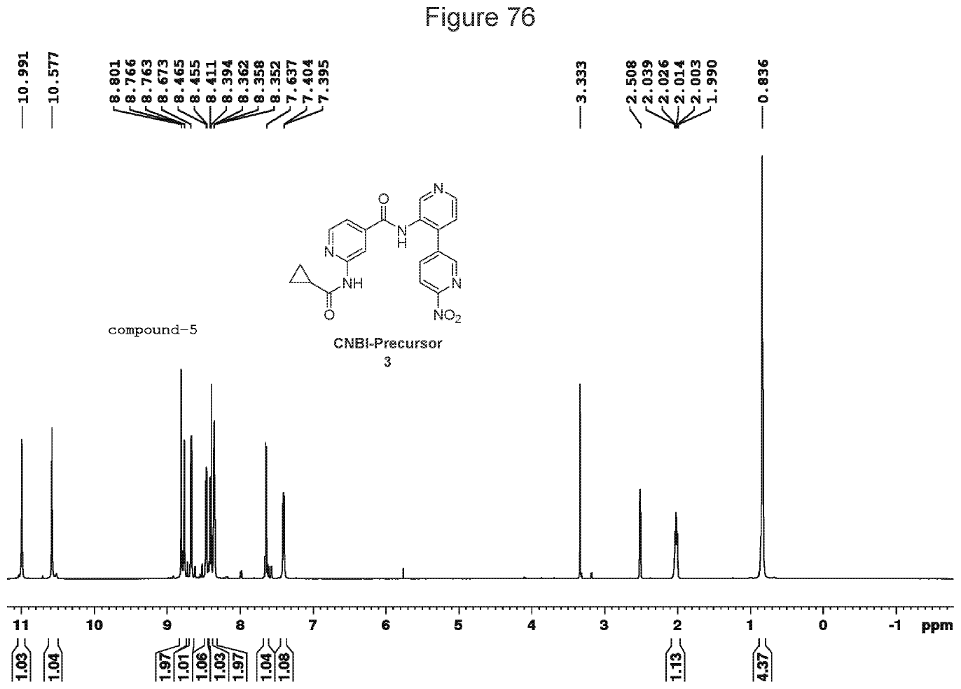

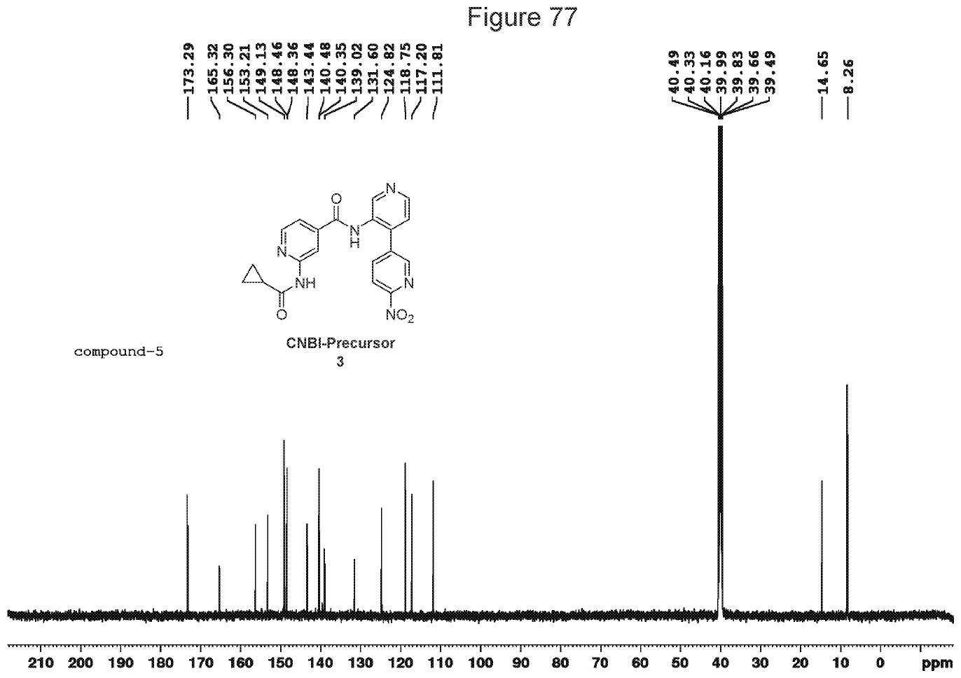

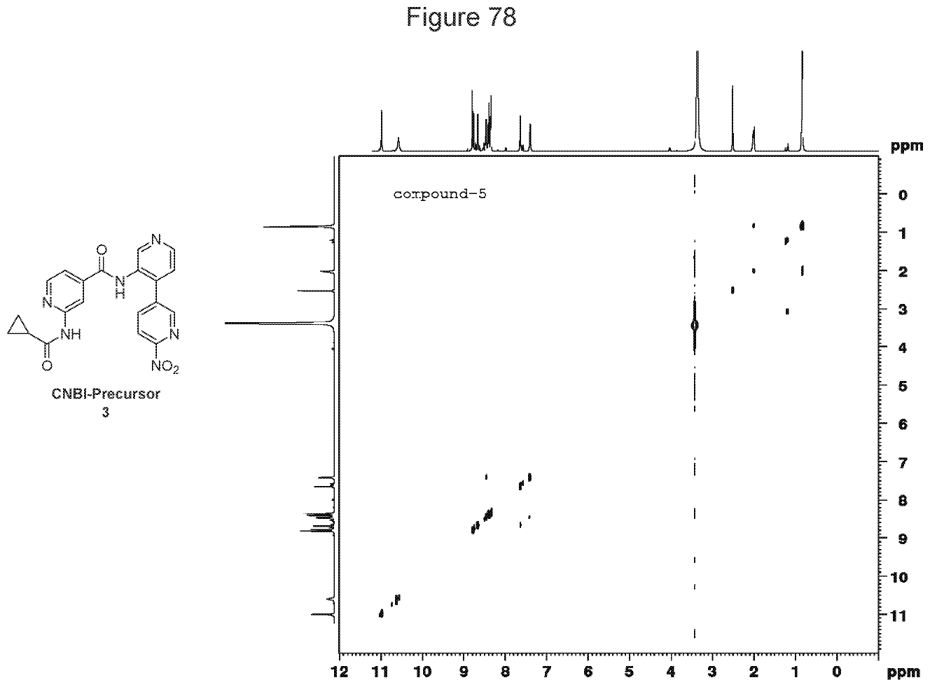

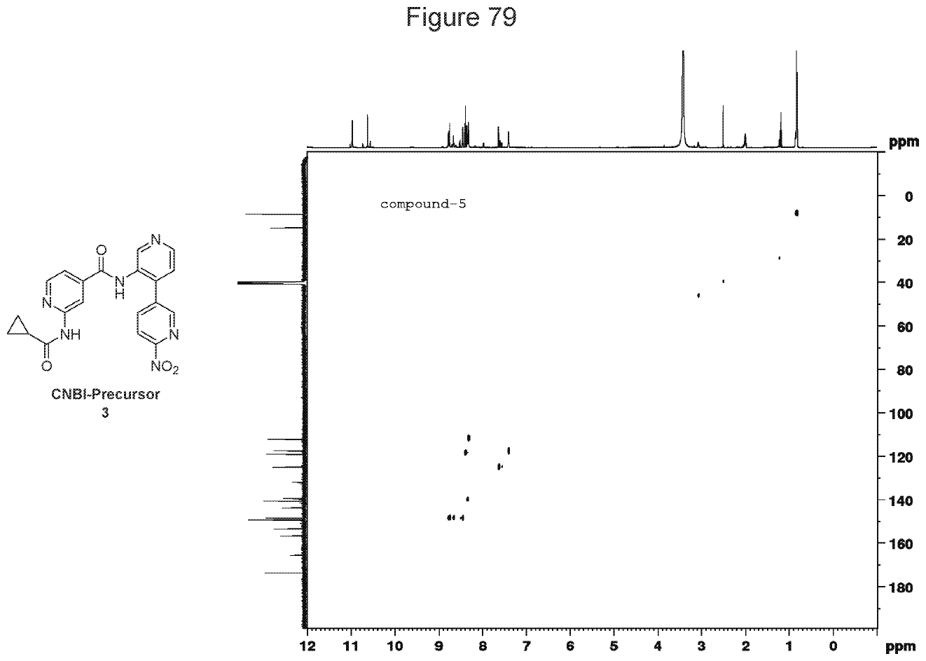

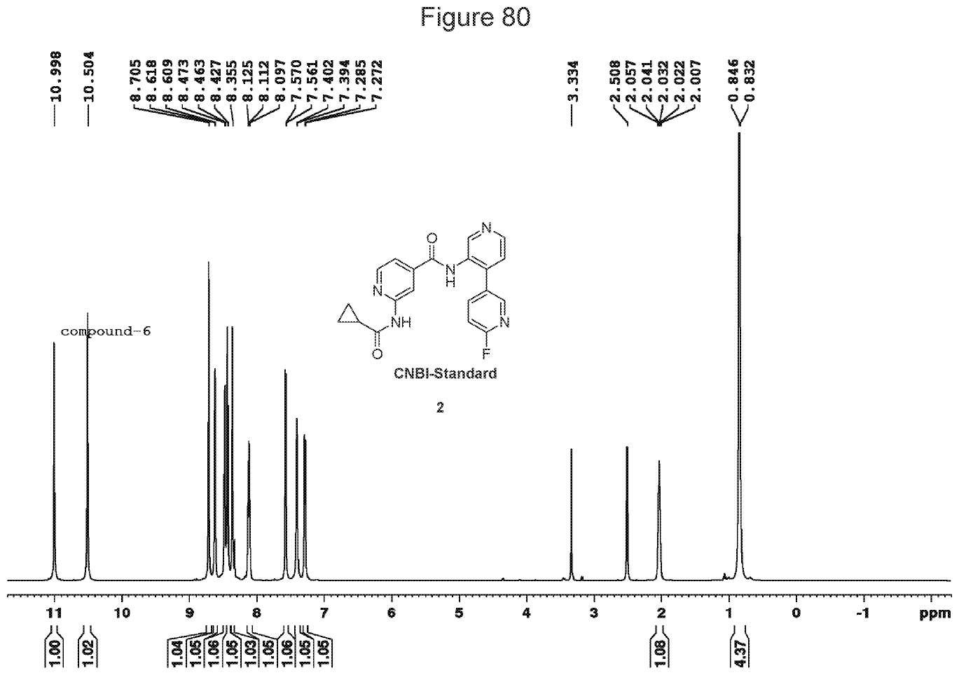

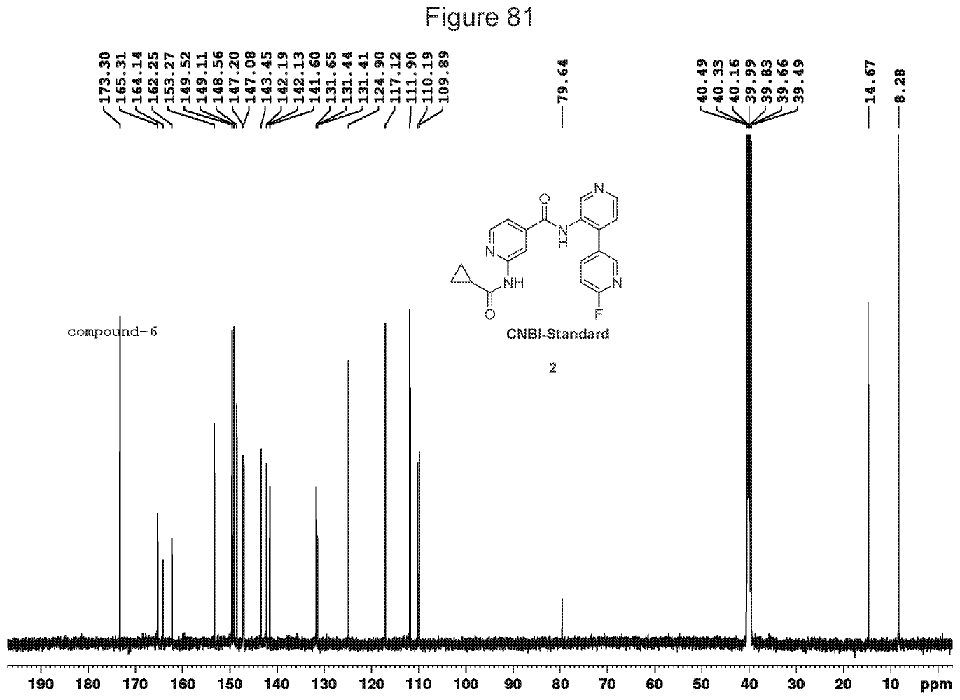

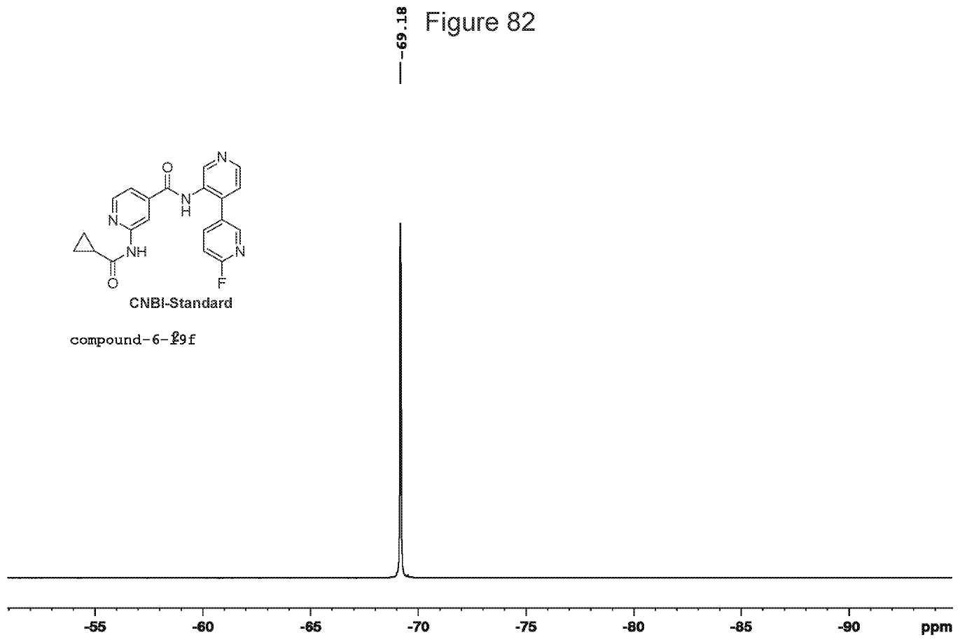

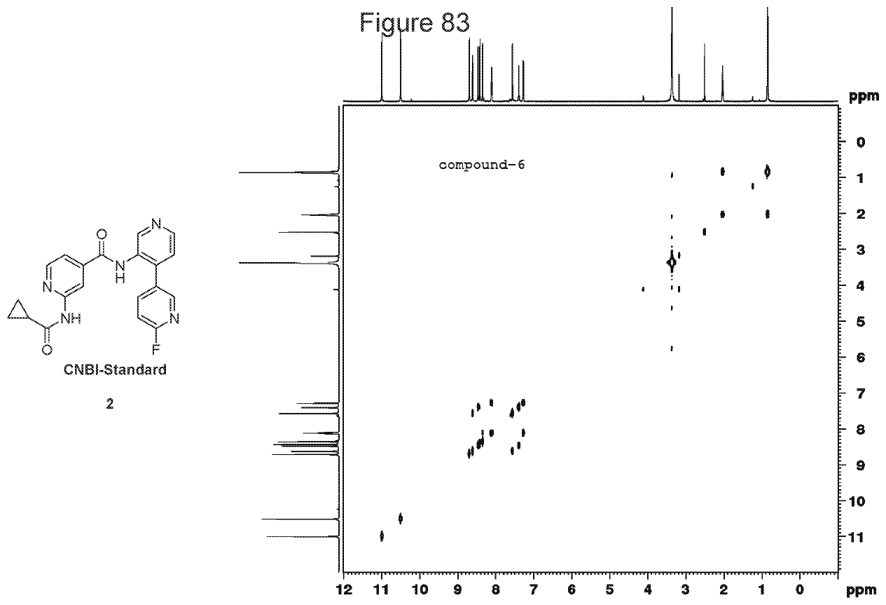

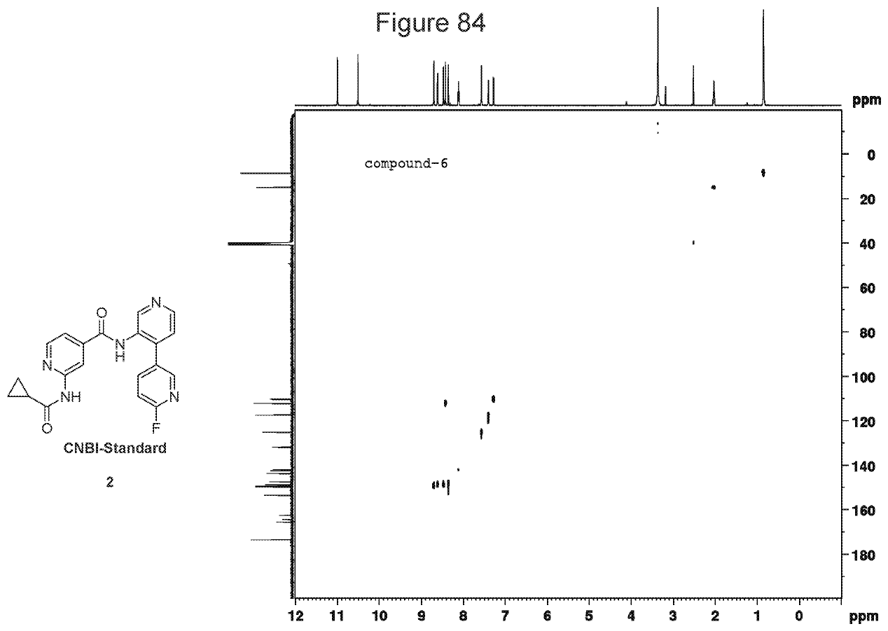

The chemical syntheses of both reference standards and precursors to produce [¹⁸F]F-CNBI and [¹⁸F]F-CNPIFE PET probes proceeded with a common intermediate 2-(cyclopropanecarboxamido)-N-(4-iodopyridin-3-yl)isonicotinamide (compound 1). The synthesis of compound 1 was reported as reacting with 4-iodopyridine-3 amine to synthesize compound 1 [Ref. 28]. Synthesis of precursor 3 and reference compound 2 were successfully synthesized through the palladium catalyzed cross-coupling reaction of 2-(cyclopropanecarboxamido)-N-(4-iodopyridin-3-yl)isonicotinamide (compound 1) with (4-nitropyridin-3-yl)boronic acid and (6-fluoropyridin-3-yl)boronic acid, respectively. The formation compound 3 was confirmed by ¹³C NMR, ¹H-¹H COSY, HSQC, and HR(EI) mass spectrometry (see FIGS. 76-79). The formation compound 2 was confirmed by ¹⁹F NMR, ¹H NMR, ¹³C NMR, ¹H-¹H COSY, HSQC, and HR(EI) mass spectrometry (see FIGS. 80-84).

Synthesis of mesylate precursor 26 for F-18 radiolabeling was performed in two steps, (i) Suzuki cross coupling, (ii) mesylation of hydroxyl moiety with methanesulfonic anhydride, first with palladium catalyzed cross-coupling reaction of 2-(cyclopropanecarboxamido)-N-(4-iodopyridin-3-yl) isonicotinamide (compound 1) with 4-(2-hydroxyethoxy)-phenyl boronic acid, which afforded a hydroxy intermediate in a decent yield. The formation of the hydroxy intermediate was confirmed by carbon NMR, and HR(EI) mass spectrometry.

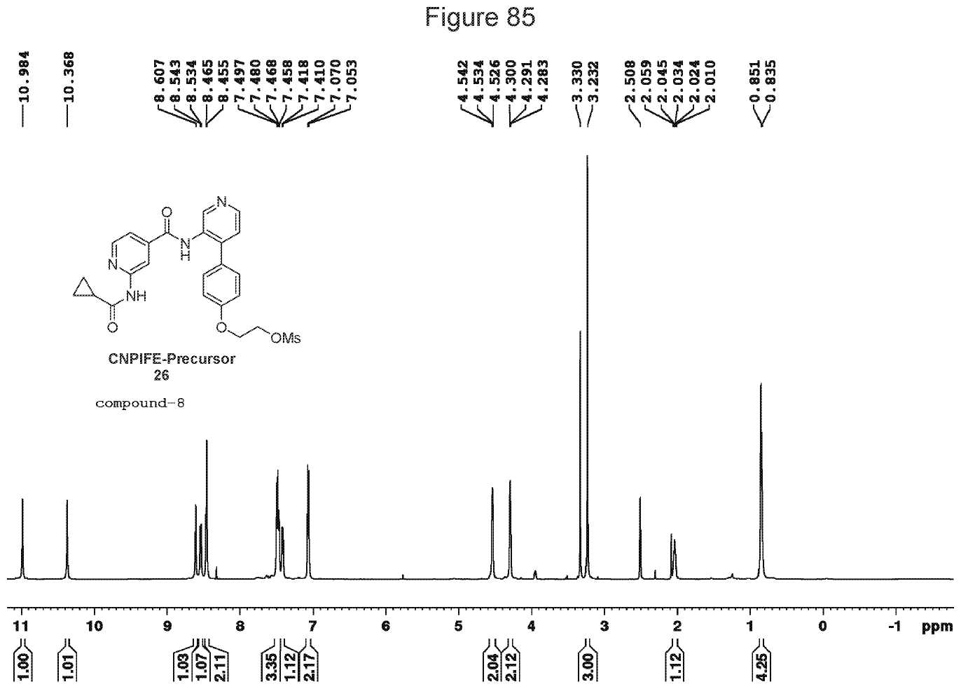

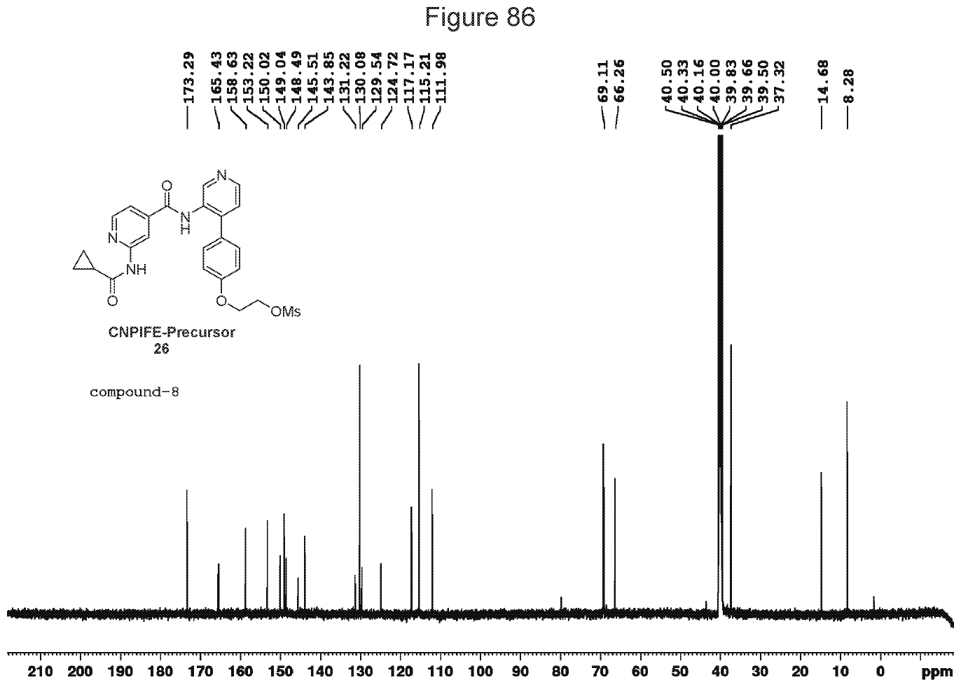

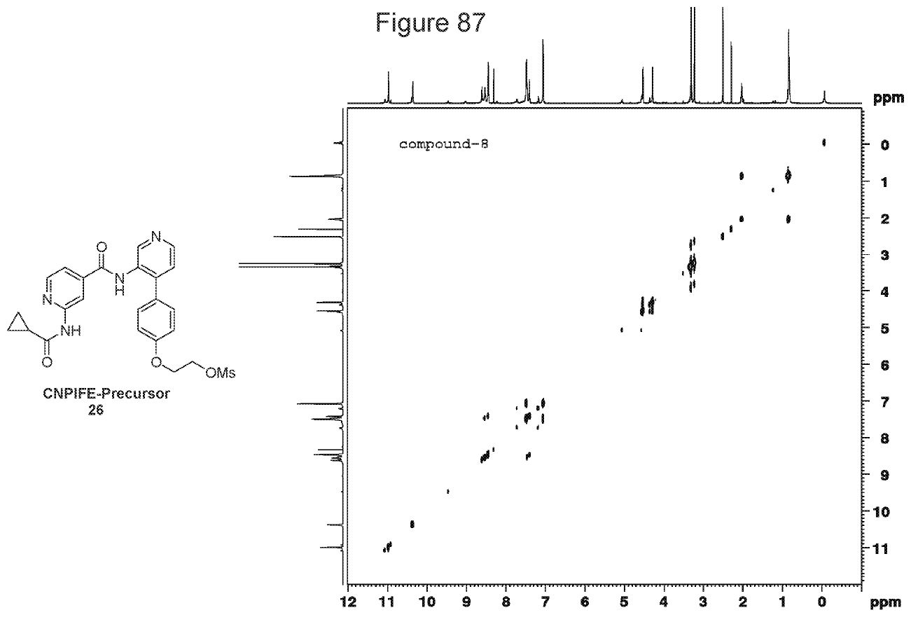

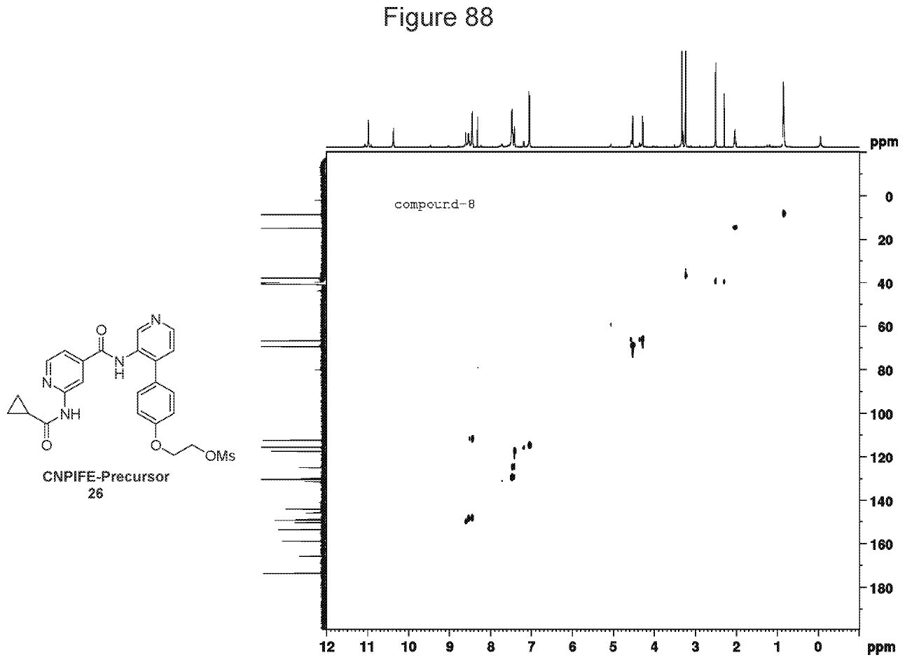

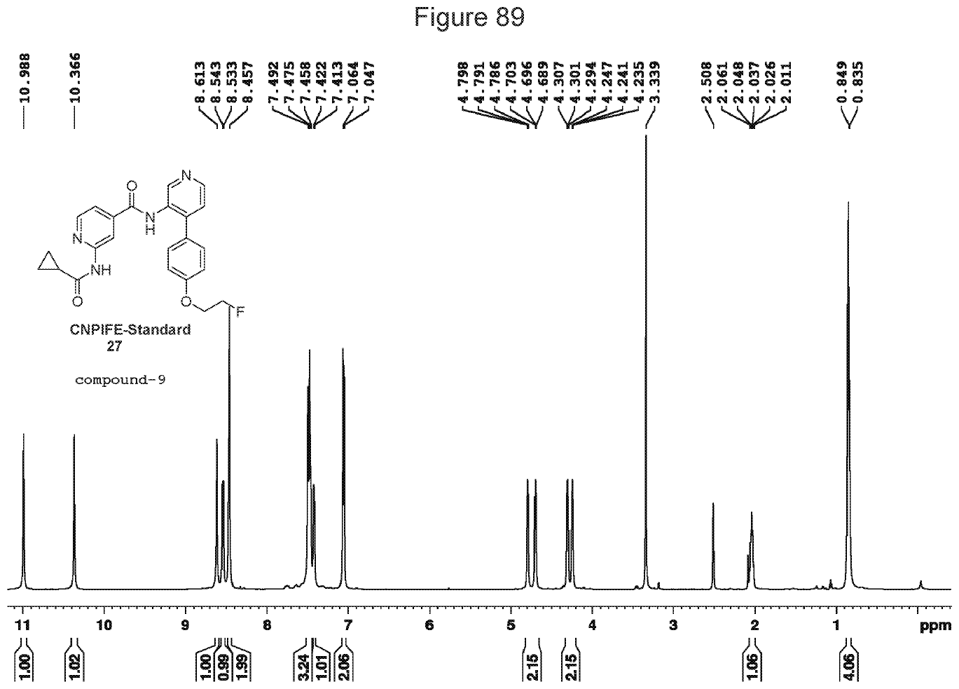

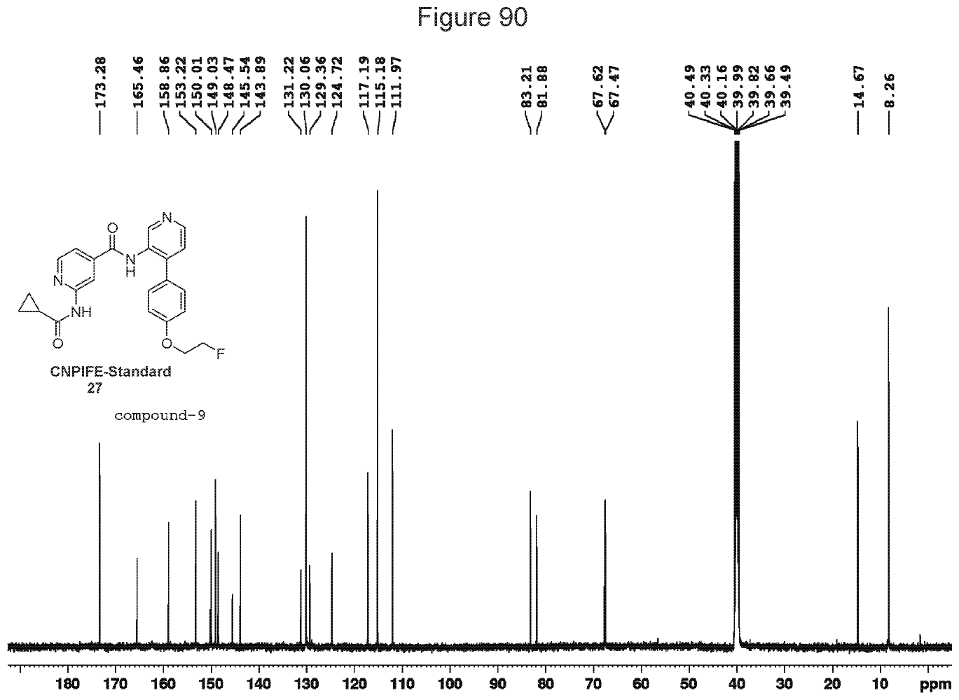

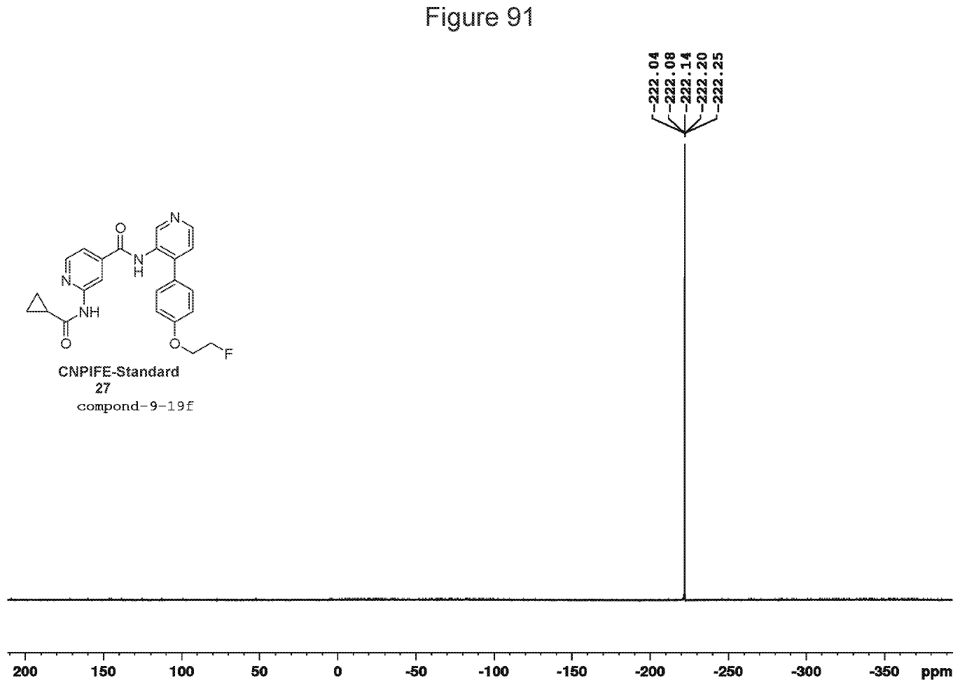

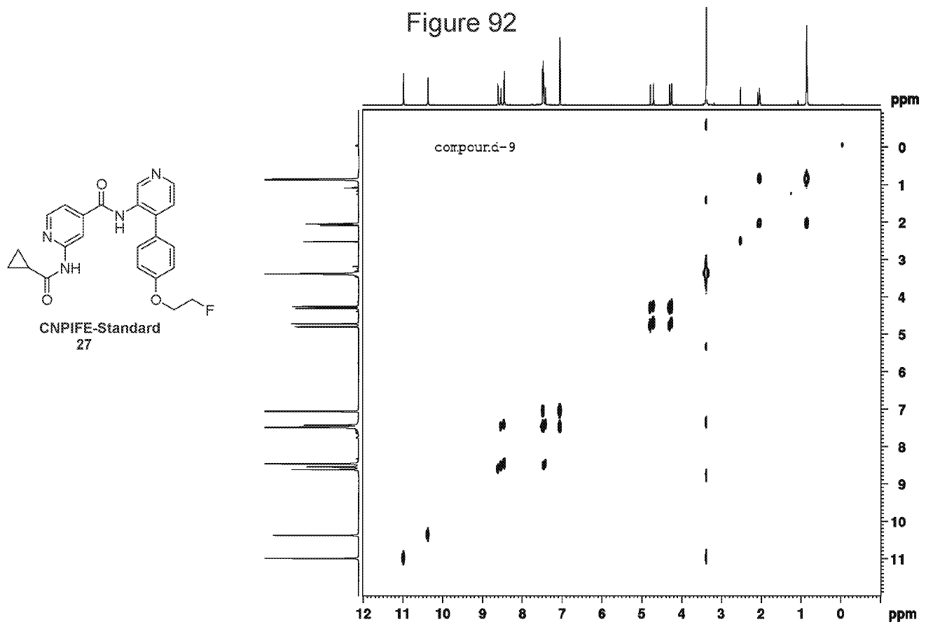

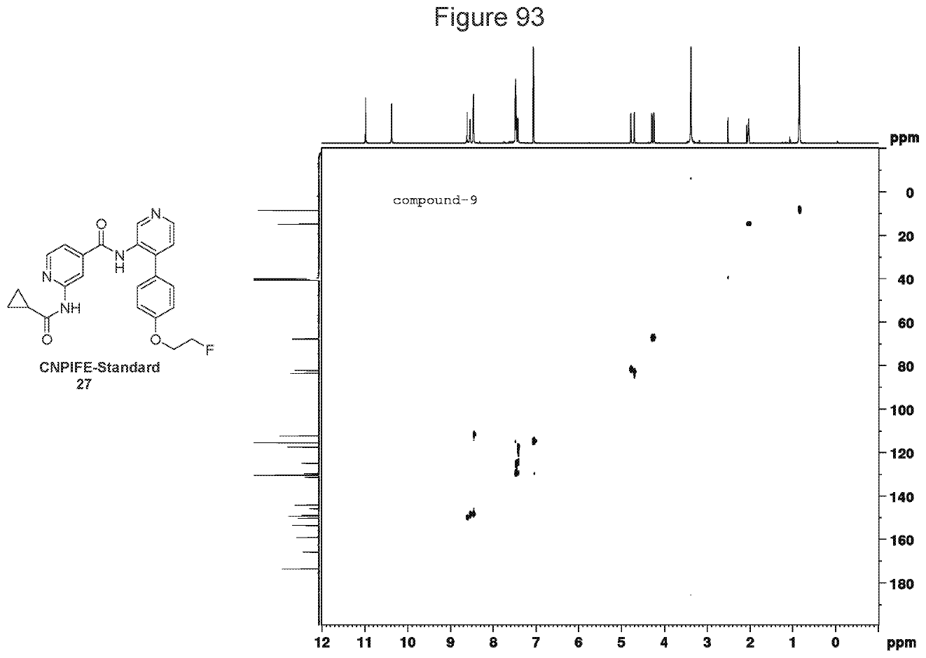

Finally, in the second step, mesylate precursor 26 was synthesized by conversion of the hydroxy group intermediate using methanesulfonic anhydride. The formation of compound 26 was characterized by the appearance of a peak in the 1H NMR spectrum (CH₂OSO₂CH₃) at δ 3.23 ppm in aliphatic region for three protons of methyl group of the mesylate moiety and by observing downfield shifts in ethylene protons of —OCH₂—CH₂O— moiety due to the formation mesylate ester of a hydroxy intermediate. The formation of compound 26 was further confirmed by ¹³C NMR, ¹H-¹H COSY, HSQC, and HR(EI) mass spectrometry (see FIGS. 85-88). For the synthesis of reference compound 27, a reported palladium catalyzed cross-coupling reaction of (4-(2-fluoroethoxy)-phenyl boronic acid with compound 1 was followed. The formation 27 was confirmed by ¹³C NMR, ¹H-¹H COSY, HSQC, and HR(EI) mass spectrometry (see FIGS. 89-92).

### Radiosynthesis

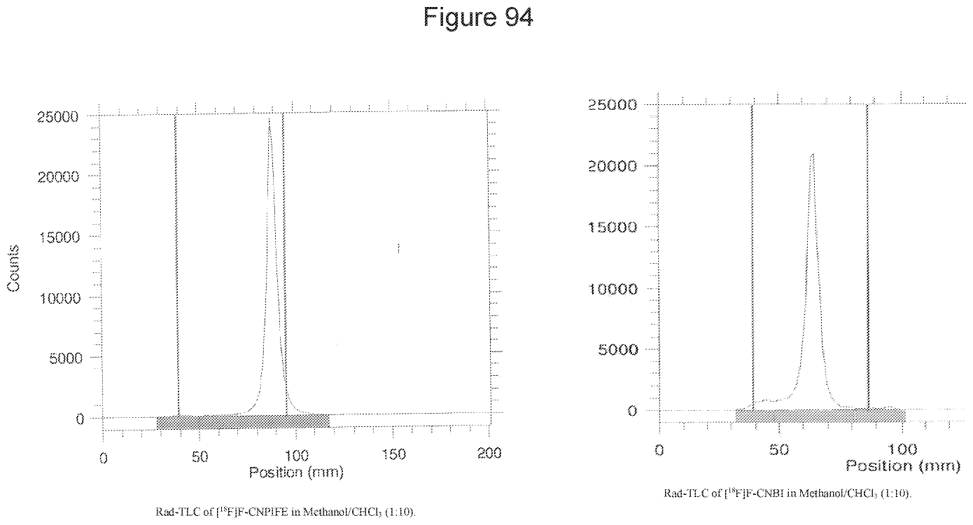

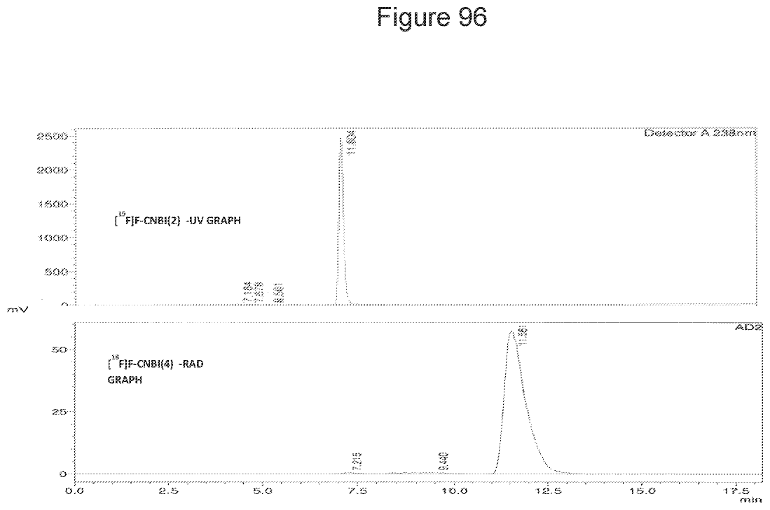

Radiosynthesis of [¹⁸F]F-CNBI and [¹⁸F]F-CNPIFE were achieved by radiofluorination of the nitro precursor, 2-(cyclopropanecarboxamido)-N-(6-nitro-[3,4′-bipyridin]-3′-yl)isonicotinamide, compound 3, and a mesylate precursor, 2-(4-(3-(2-(cyclopropanecarboxamido)isonicotinamido)pyridin-4-yl)phenoxy)ethyl methanesulfonate, compound 26, respectively. Nucleophilic [¹⁸F]fluorination was performed using cyclotron-produced [¹⁸F]fluoride using standard radio fluorination (Kryptofix 222 and K₂CO₃) reaction in a single step with 7.5±0.76% (n=3) and 8.4±5.63% (n=4) uncorrected radiolabeling yields at end of the synthesis for [¹⁸F]F-CNBI and [¹⁸F]F-CNPIFE, respectively. Radiolabeling conditions were optimized in terms of time, temperature and using 18-Crown-6 (1,4,7,10,13,16-hexaoxacyclooctadecane) as a catalyst. Radiolabeling yield was estimated using rad-TLC with a mobile phase of 10% methanol-90% chloroform (see FIG. 94). After successful radiolabeling, tracers were purified by high-performance liquid chromatography (HPLC), achieving >97% radiochemical purity. The [¹⁸F]F-CNBI and [¹⁸F]F-CNPIFE were successfully synthesized with molar activity (Aₘ) of 0.19±0.05 GBq/micromoles (n=3) and 2.2±1.7 GBq/micromoles (n=4), respectively. The HPLC spectrum of purified [¹⁸F]F-CNBI and [¹⁸F]F-CNPIFE, along with their UV traces are presented in FIGS. 95 and 96.

### In Vitro Evaluation

In vitro parallel artificial membrane permeability assay (PAMPA) was performed to estimate the BBB permeability of the unlabeled reference compounds, [¹⁹F]F-CNBI and [¹⁹F]F-CNPIFE and results are summarized in Table 1.

TABLE 1
Blood-Brain barrier permeabilities of [¹⁹F]F-CNBI
and [¹⁹F]F-CNPIFE at pH 7.4.

| | Permeability coefficient, | |
|---|---|---|
| | P (10⁻⁶ cm/s) | −logPₑ |
| Compound | Average ± SD | Average ± SD |
| [¹⁹F]F-CNBI | 1.6 ± 0.1 (n = 3) | 5.80 ± 0.02 (n = 3) |
| [¹⁹F]F-CNPIFE | 14.29 ± 0.25 (n = 5) | 4.85 ± 0.01 (n = 5) |
| Propranolol HCl (Highly | 32 ± 1 (n = 3) | 4.50 ± 0.01 (n = 3) |
| permeable control) | | |
| Atenolol (Low | <0.14 (n = 3) | — |
| permeable control) | | |

It is important to mention that a compound with higher value of permeability parameters like permeability coefficient, P (10-6 cm/s) or lower value of −log Pₑ corresponds to higher blood-brain barrier permeability. For example, a low blood-brain permeable compound atenolol, showed permeability coefficient of <0.14×10-6 cm/s (n=3), and a highly blood-brain permeable compound propranolol HCl showed permeability coefficient of 32±1×10-6 cm/s (n=4). Both [¹⁹F]F-CNBI and [¹⁹F]F-CNPIFE showed significantly higher permeability coefficients as compared to atenolol, a well-known low blood-brain permeable control compound, suggesting significantly higher BBB permeability of [¹⁹F]F-CNBI and [¹⁹F]F-CNPIFE exists than that of atenolol. Among both the reference compounds, [¹⁹F]F-CNPIFE showed a significantly higher permeability coefficient than [¹⁹F]F-CNBI. In fact, the permeability coefficient of [¹⁹F]F-CNPIFE was closer to the highly blood-brain permeable control compound propranolol than [¹⁹F]F-CNBI. Overall the PAMPA assay showed BBB permeability in the following ascending order: Atenolol<<<[¹⁹F]F-CNBI<<<[¹⁹F]F-CNPIFE<<<Propranolol.

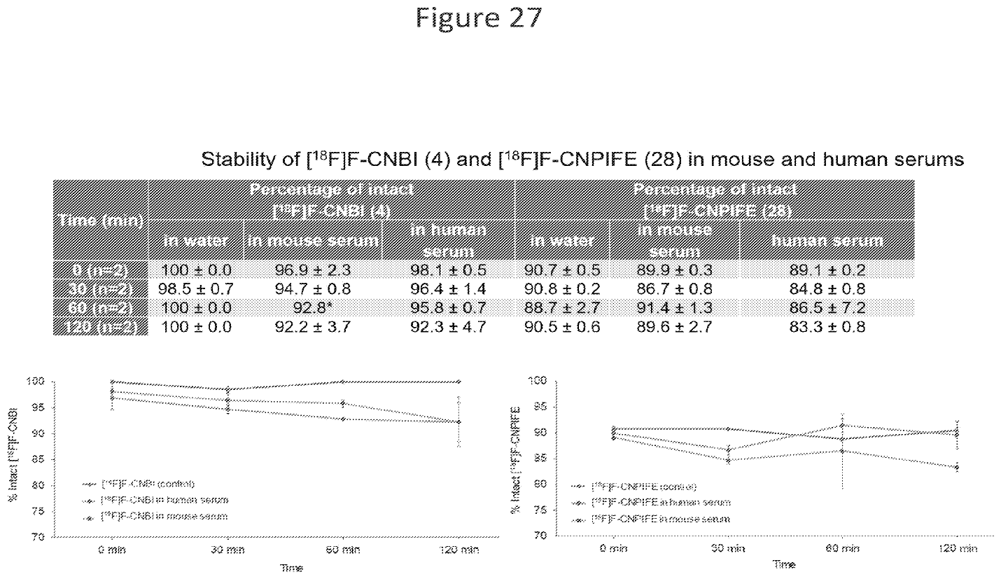

The stability of both the probes in mouse and human serums was also measured along with a control at 0 min, 30 min, 60 min, and 120 min using rad-TLC as discussed later below. It was observed that [¹⁸F]F-CNBI was stable >92% up to 120 min. However, [¹⁸F]F-CNPIFE was relatively less stable (>83%) than [¹⁸F]F-CNBI up to 120 min in both human and mouse serums as illustrated in FIG. 27.

### In Vivo PET Imaging and Biodistribution Studies

Even though the in vitro results of BBB permeability and IC₅₀ values suggested that [¹⁸F]F-CNPIFE could potentially be a better PET radiotracer for imaging of GSK-3α/β in brain as compared to the [¹⁸F]F-CNBI, it was important to understand their in vivo brain uptake and tracer kinetics in a preclinical animal model. To pursue this, [¹⁸F]F-CNBI and [¹⁸F]F-CNPIFE were evaluated in the normal FVB/NJ mouse model. To assess the brain uptake and clearance of [¹⁸F]F-CNBI and [¹⁸F]F-CNPIFE, animals were imaged dynamically for 30 min post intravenous injection of [¹⁸F]F-CNBI or [¹⁸F]F-CNPIFE in separate groups of normal FVB/NJ mice.

Significant differences in the tracer kinetic properties of [¹⁸F]F-CNBI and [¹⁸F]F-CNPIFE were observed in the brain (see FIGS. 21, 22A, 22B, and 23) and liver (see FIGS. 22C, 22D, and 24) and in whole body (see FIG. 25). The uptake of [¹⁸F]F-CNPIFE (SUV=0.48±0.09; n=5) in brain was 2.5-fold higher than [¹⁸F]F-CNBI (SUV=0.19±0.06; n=6) as early as 5 min post-injection. This difference increased with time: 4.8-fold higher SUV for [¹⁸F]F-CNPIFE (SUV=0.48±0.11; n=5) as compared to [¹⁸F]F-CNBI (SUV=0.10±0.01; n=6) at 15 min which further increased to 9.5-fold difference at 30 min (see FIG. 21). In contrast to the brain, higher uptake of [¹⁸F]F-CNBI was observed in the liver as compared to [¹⁸F]F-CNPIFE as early as 5 min post-injection, and this difference increased with time. The differences in uptakes of [¹⁸F]F-CNPIFE and [¹⁸F]F-CNBI in the brain and liver likely relate to the difference in their chemical structures (compounds 4 and 28). Greater accumulation of [¹⁸F]F-CNPIFE in the brain relative to [¹⁸F]F-CNBI was consistent with its higher BBB permeability value indicated by the PAMPA assay (see Table 1).

To further understand the characteristics of the uptake of these PET probes in the brain, self-blocking studies with corresponding non-radiolabeled reference compounds were performed. In general, co-administration of [¹⁸F]F-CNBI and [¹⁸F]F-CNPIFE with their corresponding reference compounds, [¹⁹F]F-CNBI and [¹⁹F]F-CNPIFE, respectively, were expected to decrease the uptake of radiotracers in the brain. On the contrary, co-administration of reference compounds resulted in significant increase in the brain uptake of [¹⁸F]F-CNBI, but no significant difference was observed in the brain uptake of [¹⁸F]F-CNPIFE (see FIGS. 21, 22A, 22B, and 23). However, a non-significant trend in increase in brain uptake of [¹⁸F]F-CNPIFE can be noticed on coadministration of nonradioactive [¹⁹F]F-CNPIFE with the PET probe [¹⁸F]F-CNPIFE. A difference of ˜2.5-3.0 fold uptake in [¹⁸F]F-CNBI was observed with blocking study in brain at 30 min post-injection, where brain uptake of [¹⁸F]F-CNBI was 0.08±0.005 SUV without coadministration of [¹⁹F]F-CNBI (n=6), but brain uptake increased to 0.20±0.01 SUV with coadministration of 200 μg of [¹⁹F]F-CNBI (n=2) and further increased to 0.24±0.01 SUV when co-injected with 300 μg of [¹⁹F]F-CNBI (n=2) (see FIG. 21). Additionally, an opposite trend was observed in the liver of the same group of animals showing uptake of [¹⁸F]F-CNBI as 10.15±2.87 SUV without coadministration of [¹⁹F]F-CNBI (n=6) which decreased significantly to 4.93±0.52 SUV when co-injected with 200 μg of [¹⁹F]F-CNBI (n=2) and even further decreased to 3.89±0.60 SUV when co-injected with 300 μg of [¹⁹F]F-CNBI (n=2) (see FIG. 24). We hypothesize that enhanced brain uptake with additional administration of nonradioactive form of the tracer could be attributed to the binding of [¹⁹F]F-CNBI with normally expressed GSK-3α/β in extremities and allowing relatively greater levels of [¹⁸F]F-CNBI to be taken up by the brain as a peripheral effect. In fact, a co-injection of 200-300 μg of reference compound [¹⁹F]F-CNBI with [¹⁸F]F-CNBI per mouse caused significant increase in the brain uptake of [¹⁸F]F-CNBI at all time points. Interestingly, at the same time, the uptake of [¹⁸F]F-CNBI in liver was decreased significantly at and after 15 min post-injection supporting the hypothesis of the study.

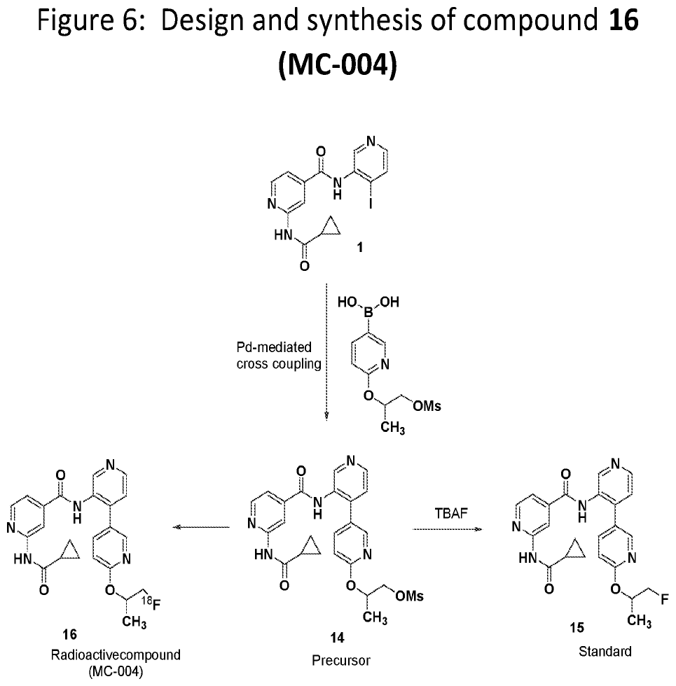

A similar trend towards higher uptake was observed with [¹⁸F]F-CNPIFE as well, and the brain uptake (SUV) of [¹⁸F]F-CNPIFE was found to be 0.76±0.16 without coadministration of [¹⁹F]F-CNPIFE (n=5) which increased to 0.88±0.14 with coadministration of 25 μg of [¹⁹F]F-CNPIFE (n=3) at 30 min post-injection (see FIG. 21). The change in uptake of [¹⁸F]F-CNPIFE in the brain and liver due to the coadministration of [¹⁹F]F-CNPIFE was not statistically significant as it was observed with [¹⁸F]F-CNBI on co-injection with [¹⁹F]F-CNBI (see FIGS. 21 and 24). This difference could be dose-dependent as only 25 μg of [¹⁹F]F— CNPIFE was co-injected per mouse as compared to 200-300 μg [¹⁹F]F-CNBI per mouse (see FIGS. 6A-6D). The lower amount of [¹⁹F]F-CNPIFE was used per mouse because of the limited aqueous solubility of [¹⁹F]F-CNPIFE in saline solution (˜25 μg/100 μL saline). Additionally, the uptake of both [¹⁸F]F-CNBI and [¹⁸F]F-CNPIFE in the brain is through a passive mechanism as there is no active transport present on the blood brain barrier for the GSK-3.

### CONCLUSION

Two PET probes, [¹⁸F]F-CNBI and [¹⁸F]F-CNPIFE, have been practically synthesized and evaluated for their affinity towards both GSK-3α and GSK-3β, and their uptakes were compared in the normal mice brain and liver over time with and without self-blocking. In vitro analysis showed nanomolar affinity of [¹⁹F]F-CNBI and [¹⁹F]F-CNPIFE towards GSK-3α and GSK-3β. Among the two, [¹⁹F]F-CNPIFE showed higher blood-brain barrier permeability than [¹⁹F]F-CNBI in a PAMPA assay. Additionally, [¹⁸F]F-CNPIFE showed 9.5-fold higher brain uptake by PET imaging compared to [¹⁸F]F-CNBI at 30 min post-injection in FVB/NJ mice. The uptake of [¹⁸F]F-CNBI was also enhanced by 2.5-3.0-fold when co-administered with reference compound [¹⁹F]F-CNBI (300 μg). Furthermore, out of the two PET probes, [¹⁸F]F-CNPIFE showed lower liver uptake and higher brain uptake than [¹⁸F]F-CNBI. The results encourage further evaluation of [¹⁸F]F-CNPIFE as a PET probe for GSK-3α/β.

### Experimental Section

General Consideration: All chemicals and solvents were purchased from various commercial manufactures (Sigma-Aldrich, Fisher Scientific, and Alfa Aesar) and used as received without further purification unless otherwise stated. TLC analysis of reaction mixtures were developed on alumina plates, compounds were visualized under UV light and/or by treatment with a solution of p-anisaldehyde followed by heating. The NMR spectra of all compounds including ¹H NMR, ¹³C NMR, DEPT, ¹⁹F NMR, ¹H-¹H Cosy and HSQC were recorded on a Bruker Ultra shield Advance III HD 500 MHz spectrometer. High-resolution mass spectra were recorded on a MICROMASS ESI-TOF MS from the University of Illinois Urbana-Champaign.

Synthesis of compounds 2-4: The synthesis of compounds 1 and 34 were performed as reported previously and characterized by matching the spectral data of the synthesized compounds with previously reported data [Ref. 28].

Procedure A—General procedure for Suzuki cross coupling: A three-necked round bottom flask attached with a water-cooled condenser was added with 2-(cyclopropanecarboxamido)-N-(4-iodopyridin-3-yl) isonicotinamide (4, 204 mg, 0.5 mmol, 1.0 equiv.) and Pd(PPh3)4 (289 mg, 0.25 mmol, 0.5 equiv.) in anhydrous tetrahydrofuran (20 mL). The mixture was allowed to react at room temperature for 45 min, followed by addition of anhydrous K₂CO₃ (67.5 mg, 2.5 mmol, 5.0 equiv.) and deionized water (0.05 mL, 2.8 mmol, 5.6 equiv.) to the reaction mixture in a sequential order. Resultant reaction mixture was then stirred for additional 20 min at room temperature followed by addition of boronic acid (1.5 equiv.) as a solid also at room temperature. After addition, the reaction mixture was heated at 80° C. with constant stirring from time ranges between 6 hours and overnight. Formation of product was monitored on silica TLC as solid phase and 2% MeOH: 98% CHCl₃ (v:v) as a mobile phase. After completion, reaction mixture was cooled to room temperature and filtered through a bunker funnel and washed with 20 mL of tetrahydrofuran. Obtained filtrate was evaporated in vacuo and the crude reaction mixture was subjected to a flash column chromatography using CHCl₃ and MeOH as mobile phase to get pure compound.

Procedure B—General procedure for preparation of compound 5: A 100 mL round bottom flask was added with 2-(cyclopropanecarboxamido)-N-(4-(4-(2-hydroxyethoxy)phenyl)pyridin-3-yl)isonicotinamide 7, (0.5 mmol, 209.08 mg, 1.0 equiv.) in 30 mL of anhydrous tetrahydrofuran, to which 0.1 mL of triethyl amine (0.75 mmol, 1.5 equiv.) and methane sulfonic anhydride (0.55 mmol, 95.81 mg, 1.1 equiv.) was added dropwise under an inert condition simultaneously. The resultant reaction mixture was stirred overnight. After completion of reaction based on TLC conversation using silica gel as a solid phase and 2% MeOH: 98% CHCl₃ as a mobile phase (Rf 0.40), tetrahydrofuran was evaporated in vacuo and the crude reaction mixture was subjected to a flash column chromatography using silica gel as a solid phase and gradient mixture of MeOH—CHCl₃ ranging from 100% CHCl₃ to 10% MeOH/90% CHCl₃ as a mobile phase to get pure compound.

Radiolabeling: For ¹⁸F-labeling, cyclotron produced ¹⁸F-fluoride was trapped on a PS—HCO3 (QMA) cartridge and eluted with 1.2 mL of eluent containing 1.5 mg of K₂CO₃ in 0.6 mL water and 10.5 mg of Kryptofix 2.2.2 in 0.6 mL acetonitrile into a 3 mL reaction vessel. This mixture was dried down under gentle stream of nitrogen at 100° C. The reaction residue was further dried by azeotropic distillation of water with anhydrous acetonitrile (3×1.0 mL). The final ¹⁸F-fluoride residue was reconstituted in 0.5 mL of anhydrous acetonitrile. For synthesis of [¹⁸F]F-CNBI, 3.0 mg of precursor compound 3 was dissolved in 1.2 mL of anhydrous DMSO with 20 mg of K₂CO₃ for 10 min in 2 mL reaction vial. Supernatant (˜1.2 mL) was collected and mixed with 3 mg of 18-Crown-6 (1,4,7,10,13,16-hexaoxacyclooctadecane) as a catalyst in a separate 3.0 mL reaction vial. Finally, 0.5 mL of the reconstituted ¹⁸F solution was added to the reaction and reaction mixture was heated for 30 min at 165° C. ¹⁸F-fluoride labeling was checked using silica r-TLC with 10% MeOH-90% CHCl₃ as a mobile phase. Final F-¹⁸ labeled [¹⁸F]F-CNBI was purified by a semipreparative HPLC (performed on a Synergy 4μ Fusion-RP 80 Å New column 20×10.0 mm at a UV detector wavelength of 238 nm. The flow rate was 2.0 mL/min. A gradient mobile phase was used 25% ACN w/0.1% TFA and 75% water w/0.1% TFA, ˜5.5-6.5 min retention time) and concentrated using a standard C-18 Sep-Pak based trap and release with 1.0 mL of ethanol and formulated in saline solution. The identity of the synthesized [¹⁸F]F-CNBI PET probe was confirmed by matching the retention time with their reference standard 6 on an analytical HPLC (performed on a Phenomenex-Jupiter, 5 μm C¹⁸(2) 300 Ao, LC Column 250×4.6 mm at a UV detector wavelength of 238 nM. The flow rate was 1.0 mL/min. A gradient mobile phase was used 25% ACN w/0.1% TFA and 75% water w/0.1% TFA, 11.8 min retention time). The molar activity (Aₘ) of [¹⁸F] F-CNBI was 0.19±0.05 GBq/micromoles (n=3) and decay corrected yield was 7.5±0.76% (n=3). Final product was formulated in isotonic NaCl solution and filtered through a 0.22 μm filter before being administered to mice.

For synthesis of [¹⁸F]F-CNPIFE, 3.0 mg of precursor compound 5 was dissolved in 1.2 mL of DMSO and reacted with 0.5 mL of the reconstituted ¹⁸F solution as described previously at 125° C. and 12 minutes. Radiosynthesis of [¹⁸F]F-CNPIFE, did not require a catalyst, and was also purified by a semipreparative HPLC (performed on a performed on a Synergy 4μ Fusion-RP 80 Å New column 20×10.0 mm at a UV detector wavelength of 238 nM. The flow rate was 1.0 mL/min. A gradient mobile phase was used 25% ACN w/0.1% TFA and 75% water w/0.1% TFA, ˜13.0-14.5 min retention time) HPLC purified [¹⁸F]F-CNPIFE was concentrated using a standard C-18 Sep-Pak based trap and release with 1.0 mL of ethanol and formulated in saline solution. The identity of the synthesized [¹⁸F]F-CNPIFE PET probe was confirmed by matching the retention time with their reference standard 6 on an analytical HPLC (performed on a Phenomenex-Jupiter, 5 μm C18(2) 300 Ao, LC Column 250×4.6 mm at a UV detector wavelength of 238 nm. The flow rate was 0.5 mL/min. A gradient mobile phase was used 25% ACN w/0.1% TFA and 75% water w/0.1% TFA, 31.6 min retention time). The developed radiosyntheses were fully automated using Trasis-All-in-one synthetic module. Automation involves sequence development for synthesis, purification, and formulation. The automated methods were tested, and a method validation was performed. The molar activity (Aₘ) of [¹⁸F]F-CNPIFE was found to be 2.2±1.7 GBq/micromoles (n=4). The overall time of synthesis was 85 minutes and decay corrected yield was 8.4±5.63% (n=4).

Blood-Brain Barrier permeability assessment: In vitro parallel artificial membrane permeability assay (PAMPA) was performed by Pion Inc., East Sussex, UK for assessing blood brain permeability of nonradioactive version of PET probes [¹⁹F]F-CNBI, [¹⁹F]F-CNPIFE, propranolol HCl and atenolol. The blood brain permeability was expressed as permeability coefficient, P (10-6 cm/s) and −log Pₑ.

Determination of IC₅₀: The phosphorylation of glycogen synthase substrate by the human GSK-3α or GSK-3β in the presence of 0-400 nM [¹⁹F]F-CNBI or [¹⁹F]F-CNPIFE was performed using human GSK-3α/β Kinase Enzyme Assay System (Promega Corporation, Madison, WI). Phosphorylation was quantified using luminescent ADP-Glo™ Kinase assay system (Promega Corporation, Madison, WI). The IC₅₀ of [¹⁹F]F-CNBI and [¹⁹F]F-CNPIFE were determined from the resulting dose response curve and IC₅₀ curve fitting tool of the GraphPad Prism 10 (GraphPad Software, San Diego, CA).

Serum Stability Analysis: Stability analysis of [¹⁸F]F-CNBI and [¹⁸F]F-CNPIFE were assessed in human and mouse serums. To perform this stability assay, 100 μL of [¹⁸F]F-CNBI (˜0.74 MBq) or 100 μL of [¹⁸F]F-CNPIFE (˜3.70 MBq) was added to 100 μL of human or mouse serum in a microcentrifuge tube. The resultant mixture of radiotracer and serum was incubated at 37° C. for 120 min. To assess the stability of the probes, a small aliquot (˜0.5 μL) was taken out from the mixture in duplicate at 0 min, 30 min, 60 min and 120 min during incubation, and analyzed on a rad-TLC using 1:9 methanol: chloroform as a mobile phase using i-TLC to estimate the radio-stability of [¹⁸F]F-CNBI or [¹⁸F]F-CNPIFE at different time points post-incubation. For control, 100 μL of [¹⁸F]F-CNBI (˜0.74 MBq) was taken in a microcentrifuge without dilution or 100 μL of [¹⁸F]F-CNPIFE (˜3.70 MBq) was added to 100 μL deionized water in a microcentrifuge tube. Both the controls, were incubated at 25° C. for 120 min. At 0 min, 30 min, 60 min and 120 min during incubation, small aliquot (˜0.5 μL) was taken from the controls in duplicate and rad-TLC was performed as mentioned above. The origin (Rf=0) represented free [¹⁸F]fluoride and solvent front (Rf=−1) represented intact [¹⁸F]F-CNBI or [¹⁸F]F-CNPIFE.

In vivo study: Biodistribution of [¹⁸F]F-CNBI and [¹⁸F]F-CNPIFE were assessed in brain and in liver of normal FVB/NJ mice (The Jackson Laboratory, Bar Harbor, ME) with and without co administration of unlabeled reference compounds [¹⁹F]F-CNBI (200 μg and 300 μg) and [¹⁹F]F-CNPIFE (25 μg) in separate groups of animals. Stock solutions of [¹⁹F]F-CNPIFE (26 mM) and [¹⁹F]F-CNBI (25 mM) were prepared in the dimethyl sulfoxide (DMSO). For blocking studies with [¹⁹F]F-CNBI, 200 μg (530.5 nmol) or 300 μg (795.8 nmol) of [¹⁹F]F-CNBI (nonradioactive reference compound) was added to the PET probe [¹⁸F]F-CNBI self-containing ˜9.3 nmol [¹⁹F]F-CNBI in the final formulation volume of ˜100 μL. For blocking with [¹⁹F]F-CNPIFE, 25 μg (59 nmol) of [¹⁹F]F-CNPIFE was added to the PET probe [¹⁸F]F-CNPIFE self-containing to 0.8-1.0 nmol of [¹⁹F]F-CNPIFE in the final formulation volume of ˜100 μL. The final formulation containing both PET probes and their nonradioactive reference compounds were prepared in 0.9% saline isotonic solution before tail vein injection. Immediately after the tail vein injection of the radiotracers, animals were subjected to a dynamic micro-PET imaging for 30 min using a small animal micro-PET/X-ray system (Sofie BioSystems Genesys4, Culver City, CA, USA). The PET images were visualized and analyzed using image analysis software, Amide's a Medical Image Data Examiner—AMIDE [Ref. 29]. PET images at 5 min, 10 min, 15 min, 20 min, 25 min, and 30 min post-injection were normalized to the standardized uptake value (SUV), where SUV=(Concentration of dose in tissue (μCi/g)/(Injected dose (μCi)/Weight of the animal (g)) and presented as coronal, transverse and sagittal sectional image.

### REFERENCES

1. O. Kaidanovich-Beilin, J. R. Woodgett, Front. Mol. Neurosci. 2011, 4, 40.
2. F. Takahashi-Yanaga, Biochem. Pharmacol. 2013, 86, 191-199.
3. J. Lee, M. S. Kim, Diabetes Res Clin Pract. 2007, 77, S49-S57.
4. M. Lesort, A. Greendorfer, C. Stockmeier, G. V. W. Johnson, J. Neural Transm. 1999, 106, 1217-1222.
5. A. J. Alvezan, P. S. Klein, Front. Mol. Neurosci. 2012, 5, 1.
6. J. Luo, Cancer Lett. 2009, 273, 194-200.
7. M. Momcilovic, S. T. Bailey, J. T. Lee, M. C. Fishbein, D. Braas, J. Go, T. G. Graeber, F. Parlati, S. Demo, R. Li, T. C. Walser, M. Gricowski, R. Shuman, J. Ibarra, D. Fridman, M. E. Phelps, K. Badran, M. S. John, N. M. Bernthal, N. Federman, J. Yanagawa, S. M. Dubinett, S. Sadeghi, H. R. Christoff, D. B. Shackelford, Cancer Cell. 2018, 33, 905-921.
8. D. W. Maixner, H. R. Weng, J. Pharm. Pharmacol. 2013, 1, 001.
9. K. Leroy, Z. Yilmaz, J. P. Brion, Neuropathol App/Neurobiol. 2007, 33, 43-55.
10. P. Lei, S. Ayton, A. I. Bush, P. A. Adlard, Int. J. Alzheimer's Dis. 2011, 2011, 189246.
11. A. Cavallini, S. Brewerton, A. Bell, S. Sargent, S. Glover, C. Hardy, R. Moore, J. Calley, D. Ramachandran, M. Poidinger, E. Karran, P. Davies, M. Hutton, P. Szekeres, S. Bose, J Biol Chem. 2013, 288, 23331-23347.
12. J. Avila, F. Wandosell, F. Hernendez, Expert Rev. Neurother. 2010, 10, 703-710.
13. C. Hooper, R. Killick, S. J. Lovestone, Neurochem. 2008, 104, 1433-1439.
14. A. V. Mossine, E. L. Cole, A. F. Brooks, L. Li, F. Aigbirhio, P. J. Scott, J Label Compd Radiopharm. 2015, 58, S411.
15. A. Mossine, L. Lie, E. Cole, A. Brooks, F. Aigbirhio, P. Scott, J Nucl Med. 2015, 56, 1094-1099.
16. M. Wang, M. Gao, K. D. Miller, G. W. Sledge, G. D. Hutchins, Q. H. Zheng, Bioorg MedChem Lett. 2011, 21, 245-249.
17. L. Li, X. Shao, E. L. Cole, S. A. Ohnmacht, V. Ferrari, Y. T. Hong, D. J. Williamson, T. D. Fryer, C. A. Quesada, P. Sherman, P. J. Riss, P. J. H. Scott, F. I. Aigbirhio, ACS Med ChemLett. 2015, 6, 548-552.
18. N. Vasdev, A. Garcia, W. T. Stableford, A. B. Young, J. H. Meyer, S. Houle, A. A. Wilson, Bioorg Med Chem Lett. 2005, 15, 5270-5273.
19. J. W. Hicks, A. A. Wilson, E. A. Rubie, J. R. Woodgett, S. Houle, N. Vasdev, Bioorg MedChem Lett. 2012, 22, 2099-2101.
20. S. Berg, M. Bergh, S. Hellberg, K. Hogdin, Y. Lo-Alfredsson, P. Soderman, S. V. Berg, T. Weigelt, M. Ormö, Y. Xue, J. Tucker, J. Neelissen, E. Jerning, Y. Nilsson, R. Bhat, J MedChem. 2012, 55, 9107-9119.
21. E. L. Cole, X. Shao, P. Sherman, C. Quesada, M. V. Fawaz, T. J. Desmond, P. J, H. Scott, Nucl Med Biol. 2014, 41, 507-512.
22. A. V. Mossine, A. F. Brooks, I. M. Jackson, C. A. Quesada, P. Sherman, E. L. Cole, D. J. Donnelly, P. J. H. Scott, X. Shao, Bioconjug Chem. 2016, 27, 1382-1389.
23. J. Prabhakaran, F. Zanderigo, K. K. S. Sai, H. Rubin-Falcone, M. J. Jorgensen, J. R. Kaplan, A. Mintz, J. J. Mann, J. S. D. Kumar, ACS Chem Neurosci. 2017, 8, 1697-1703.
24. S. Liang, J. Chen, M. Normandin, T. Collier, R. Perils, E. Holson, S. Haggarty, G. El Fakhri, R. Kurumbail, N. Vasdev, J Nucl Med. 2015, 56, 491.
25. D. Terwel, D. Muyllaert, I. Dewachter, P. Borghgraef, S. Croes, H. Devijver, F. VanLeuven, Am J Pathol. 2008, 172, 786-798.
26. A. J. Shepherd, M. E. Cloud, Y. Q. Cao, D. P. Mohapatra, Front Behav Neurosci. 2018, 28, 124.
27. C. F. Xia, J. Arteaga, G. Chen, U. Gangadharmath, L. F. Gomez, D. Kasi, C. Lam, Q. Liang, C. Liu, V. P. Mocharla, F. Mu, A. Sinha, H. Su, A. K. Szardenings, J. C. Walsh, E. Wang, C. Yu, W. Zhang, T. Zhao, H. C. Kolb, Alzheimer's Dement. 2013, 9, 666-676.
28. G. Luo, L. Chen, C. R. Burton, H. Xiao, P. Sivaprakasam, C. M. Krause, Y. Cao, N. Liu, J. Lippy, W. J. Clarke, K. Snow, J. Raybon, V. Arora, M. Pokross, K. Kish, H. A. Lewis, D. R. Langley, J. E. Macor, G. M. Dubowchik, J Med Chem. 2016, 59, 1041-1051.
29. A. M. Loening, S. S. Gambhir, Mol Imaging. 2003, 2, 131-137.
   The citation of any document or reference is not to be construed as an admission that it is prior art with respect to the present invention.

Thus, the invention provides selective and blood brain barrier permeable positron emission tomography imaging probes.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A compound of formula (1): 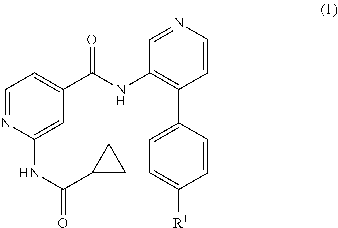
wherein R¹ is selected from the group consisting of unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, substituted alkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and
wherein at least one atom in R¹ is replaced with a positron emitter.

2. The compound of claim 1 wherein R¹ is substituted amino.

3. The compound of claim 1 wherein R¹ is unsubstituted sulfonyloxy or substituted sulfonyloxy.

4. The compound of claim 1 wherein:
the positron emitter is selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y ⁸⁹Zr, ⁹⁰Nb, ^{94m}Tc, ^{110m}In, ¹¹⁸Sb, ¹²⁰I, ¹²¹I, ¹²²I, and ¹²⁴I.

5. The compound of claim 4 wherein the compound has the formula (51):

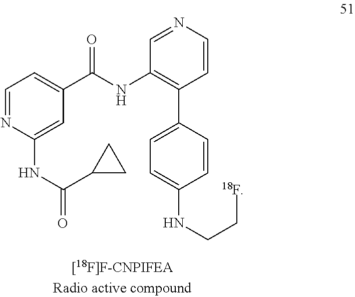

6. The compound of claim 4 wherein the compound has the formula (54):

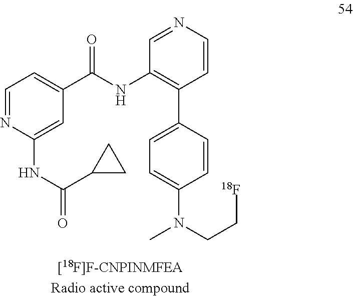 [SMILES: CN(CC[18F])c1ccc(-c2ccncc2NC(=O)c2ccnc(NC(=O)C3CC3)c2)cc1]

7. The compound of claim 4 wherein the compound has the formula (56):

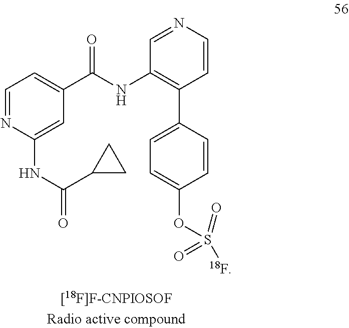

8. The compound of any of claims 1 to 7 wherein:
the compound is capable of binding to GSK-3.

9. The compound of any of claims 1 to 7 wherein:
the compound is capable of specific binding to GSK-3.

10. The compound of any of claims 1 to 7 wherein:
the compound is capable of specific binding to GSK-3α and GSK-3β.

11. The compound of any of claims 1 to 7 wherein:
the compound does not bind with proteins that compete with GSK-3α and GSK-3β.

12. The compound of any of claims 1 to 7 wherein:
the compound exhibits blood brain barrier (BBB) penetration.

13. A compound of formula (2): 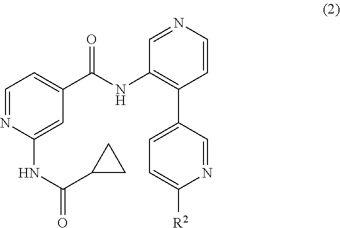
wherein R² is selected from the group consisting of unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and
wherein at least one atom in R² is replaced with a positron emitter.

14. The compound of claim 13 wherein R² is unsubstituted alkoxy.

15. The compound of claim 13 wherein R² is substituted alkoxy.

16. The compound of claim 13 wherein R² is substituted amino.

17. The compound of claim 13 wherein:
the positron emitter is selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y ⁸⁹Zr, ⁹⁰Nb, ^{94m}Tc, ^{110m}In, ¹¹⁸Sb, ¹²⁰I, ¹²¹I, ¹²²I, and ¹²⁴I.

18. The compound of claim 13 wherein the compound has the formula (7a): 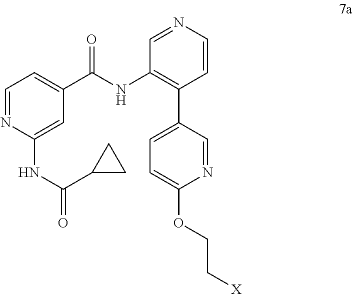
wherein X is the positron emitter.

19. The compound of claim 13 wherein the compound has the formula (7):

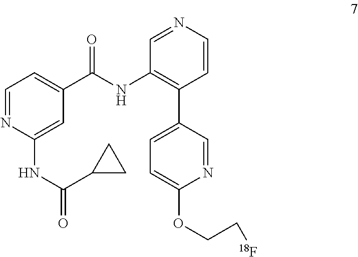 [SMILES: O=C(Nc1cnccc1-c1ccc(OCC[18F])nc1)c1ccnc(NC(=O)C2CC2)c1]

20. The compound of claim 13 wherein the compound has the formula (10):

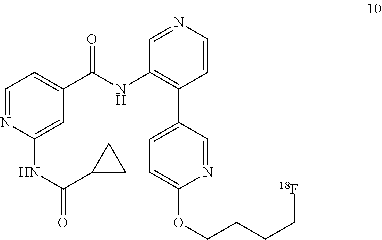 [SMILES: O=C(Nc1cnccc1-c1ccc(OCCCC[18F])nc1)c1ccnc(NC(=O)C2CC2)c1]

21. The compound of claim 13 wherein the compound has the formula (13):

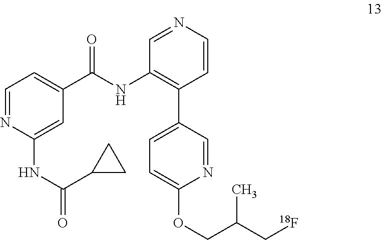 [SMILES: CC(C[18F])COc1ccc(-c2ccncc2NC(=O)c2ccnc(NC(=O)C3CC3)c2)cn1]

22. The compound of claim 13 wherein the compound has the formula (16):

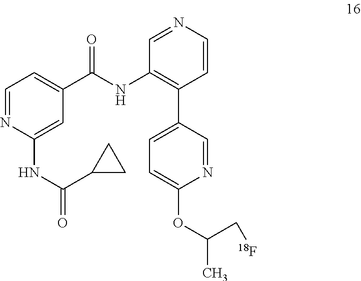 [SMILES: CC(C[18F])Oc1ccc(-c2ccncc2NC(=O)c2ccnc(NC(=O)C3CC3)c2)cn1]

23. The compound of claim 13 wherein the compound has the formula (19):

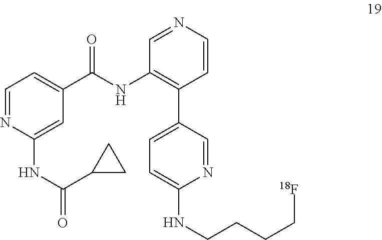 [SMILES: O=C(Nc1cnccc1-c1ccc(NCCCC[18F])nc1)c1ccnc(NC(=O)C2CC2)c1]

24. The compound of claim 13 wherein the compound has the formula (22):

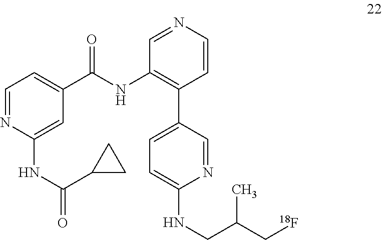 [SMILES: CC(C[18F])CNc1ccc(-c2ccncc2NC(=O)c2ccnc(NC(=O)C3CC3)c2)cn1]

25. The compound of claims 13 to 24 wherein:
the compound is capable of binding to GSK-3.

26. The compound of claims 13 to 24 wherein:
the compound is capable of specific binding to GSK-3.

27. The compound of claims 13 to 24 wherein:
the compound is capable of specific binding to GSK-3α and GSK-3β.

28. The compound of claims 13 to 24 wherein:
the compound does not bind with proteins that compete with GSK-3α and GSK-3β.

29. The compound of claims 13 to 24 wherein:
the compound exhibits blood brain barrier (BBB) penetration.

30. A compound of formula (75): 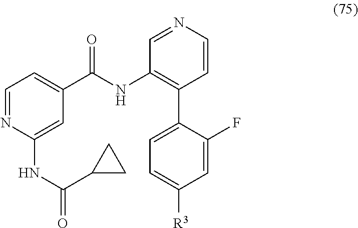
wherein R³ is selected from the group consisting of hydrogen, unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and
wherein at least one atom in R³ is replaced with a positron emitter.

31. The compound of claim 30 wherein R³ is hydrogen.

32. The compound of claim 30 wherein:
the positron emitter is selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y ⁸⁹Zr, ⁹⁰Nb, ^{94m}Tc, ^{110m}In, ¹¹⁸Sb, ¹²⁰I, ¹²¹I, ¹²²I, and ¹²⁴I.

33. The compound of claim 30 wherein the compound has the formula (59):

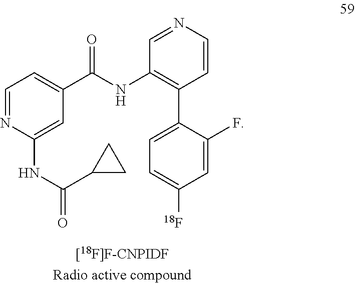 [SMILES: O=C(Nc1cnccc1-c1ccc([18F])cc1F)c1ccnc(NC(=O)C2CC2)c1]

34. The compound of claims 30 to 33 wherein:
the compound is capable of binding to GSK-3.

35. The compound of claims 30 to 33 wherein:
the compound is capable of specific binding to GSK-3.

36. The compound of claims 30 to 33 wherein:
the compound is capable of specific binding to GSK-3α and GSK-3β.

37. The compound of claims 30 to 33 wherein:
the compound does not bind with proteins that compete with GSK-3α and GSK-3β.

38. The compound of claims 30 to 33 wherein:
the compound exhibits blood brain barrier (BBB) penetration.

39. A compound of formula (80): 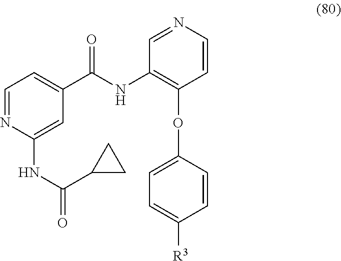
wherein R⁴ is selected from the group consisting of hydrogen, unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and
wherein at least one atom in R⁴ is replaced with a positron emitter.

40. The compound of claim 39 wherein R⁴ is hydrogen.

41. The compound of claim 39 wherein R⁴ is unsubstituted alkoxy.

42. The compound of claim 39 wherein:
the positron emitter is selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y ⁸⁹Zr, ⁹⁰Nb, ^{94m}Tc, ^{110m}In, ¹¹⁸Sb, ¹²⁰I, ¹²¹I, ¹²²I, and ¹²⁴I.

43. The compound of claim 39 wherein the compound has the formula (63):

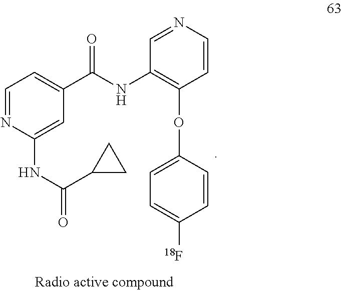 [SMILES: O=C(Nc1cnccc1Oc1ccc([18F])cc1)c1ccnc(NC(=O)C2CC2)c1]

44. The compound of claim 39 wherein the compound has the formula (66):

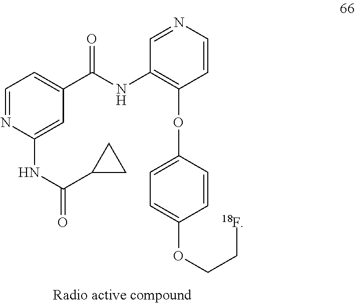

45. The compound of claims 39 to 44 wherein:
the compound is capable of binding to GSK-3.

46. The compound of claims 39 to 44 wherein:
the compound is capable of specific binding to GSK-3.

47. The compound of claims 39 to 44 wherein:
the compound is capable of specific binding to GSK-3α and GSK-3β.

48. The compound of claims 39 to 44 wherein:
the compound does not bind with proteins that compete with GSK-3α and GSK-3β.

49. The compound of claims 39 to 44 wherein:
the compound exhibits blood brain barrier (BBB) penetration.

50. A compound of formula (90): 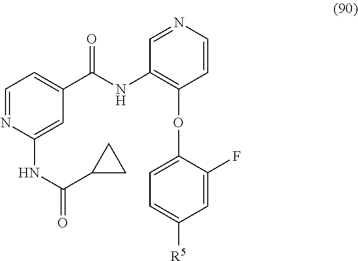
wherein R⁵ is selected from the group consisting of hydrogen, unsubstituted alkyl, substituted alkyl, unsubstituted haloalkyl, substituted haloalkyl, unsubstituted hydroxyalkyl, substituted hydroxyalkyl, benzyl, phenyl, substituted phenyl, unsubstituted alkenyl, substituted alkenyl, unsubstituted cycloalkyl, substituted cycloalkyl, hydroxy, unsubstituted alkoxy, substituted alkoxy, unsubstituted haloalkoxy, substituted haloalkoxy, unsubstituted phenoxy, substituted phenoxy, unsubstituted sulfonyloxy, substituted sulfonyloxy, carbonyl, carboxy, unsubstituted amino, substituted amino, unsubstituted amido, and substituted amido, and
wherein at least one atom in R⁵ is replaced with a positron emitter.

51. The compound of claim 50 wherein R⁵ is hydrogen.

52. The compound of claim 50 wherein R⁵ is unsubstituted alkoxy.

53. The compound of claim 50 wherein:
the positron emitter is selected from the group consisting of ¹¹C, ¹³N, ¹⁵O, ¹⁸F, ^{34m}Cl, ³⁸K, ⁴⁵Ti, ⁵¹Mn, ^{52m}Mn, ⁵²Fe, ⁵⁵Co, ⁶⁰Cu, ⁶¹Cu, ⁶²Cu, ⁶⁴Cu, ⁶⁶Ga, ⁶⁸Ga, ⁷¹As, ⁷²As, ⁷⁴As, ⁷⁵Br, ⁷⁶Br, ⁸²Rb, ⁸⁶Y ⁸⁹Zr, ⁹⁰Nb, ^{94m}Tc, ^{110m}In, ¹¹⁸Sb, ¹²⁰I, ¹²¹I, ¹²²I, and ¹²⁴I.

54. The compound of claim 54 wherein the compound has the formula (69):

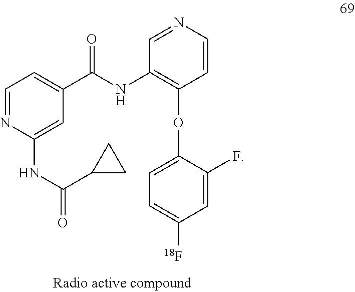 [SMILES: O=C(Nc1cnccc1Oc1ccc([18F])cc1F)c1ccnc(NC(=O)C2CC2)c1]

55. The compound of claim 54 wherein the compound has the formula (72):

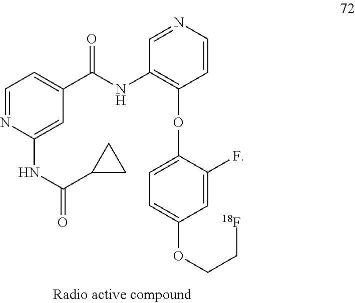 [SMILES: O=C(Nc1cnccc1Oc1ccc(OCC[18F])cc1F)c1ccnc(NC(=O)C2CC2)c1]

56. The compound of claims 50 to 55 wherein:
the compound is capable of binding to GSK-3.

57. The compound of claims 50 to 55 wherein:
the compound is capable of specific binding to GSK-3.

58. The compound of claims 50 to 55 wherein:
the compound is capable of specific binding to GSK-3α and GSK-3β.

59. The compound of claims 50 to 55 wherein:
the compound does not bind with proteins that compete with GSK-3α and GSK-3β.

60. The compound of claims 50 to 55 wherein:
the compound exhibits blood brain barrier (BBB) penetration.

61. A method for in vivo imaging of a subject, the method comprising:
(a) administering to the subject the compound of any of claims 1 to 7, or 13 to 24, or 30 to 33, or 39 to 44, or 50 to 55;
(b) waiting a time sufficient to allow the compound to accumulate at a tissue or cell site to be imaged; and
(c) imaging the cells or tissues with a non-invasive imaging technique.

62. The method of claim 61 wherein:
the non-invasive imaging technique is selected from positron emission tomography imaging, positron emission tomography with computed tomography imaging, or positron emission tomography with magnetic resonance imaging.

63. A method of imaging a subject by positron emission tomography, the method comprising:
(a) administering the compound of any of claims 1 to 7, or 13 to 24, or 30 to 33, or 39 to 44, or 50 to 55 to the subject;
(b) using a plurality of detectors to detect gamma rays emitted from the subject and to communicate signals corresponding to the detected gamma rays; and
(c) reconstructing from the signals a series of medical images of a region of interest of the subject.

64. An imaging method comprising acquiring an image of a subject to whom a detectable amount of the compound of any of claims 1 to 7, or 13 to 24, or 30 to 33, or 39 to 44, or 50 to 55 has been administered.

65. The method of claim 64, which comprises acquiring a brain image of the subject.

66. The method of claim 64, which comprises acquiring a liver image of the subject.

67. The method of claim 64, which comprises acquiring the image using positron emission tomography imaging, positron emission tomography with computed tomography imaging, or positron emission tomography with magnetic resonance imaging.

68. The method of claim 64, wherein the detectable amount of the compound is an amount of the compound that is sufficient to enable detection of accumulation of the compound in cells or tissue by a medical imaging technique.

69. A method for detecting GSK-3 in a subject, the method comprising:
(a) administering to the subject the compound of any of claims 1 to 7, or 13 to 24, or 30 to 33, or 39 to 44, or 50 to 55;
(b) waiting a time sufficient to allow the compound to accumulate at a tissue or cell site to be imaged; and
(c) imaging the cells or tissues with a non-invasive imaging technique.

70. The method of claim 69 wherein:
the tissue or cell site is in the brain.

71. The method of claim 69 wherein:
the tissue or cell site is in the liver.

72. The method of claim 69 wherein:
the non-invasive imaging technique is selected from positron emission tomography imaging, positron emission tomography with computed tomography imaging, or positron emission tomography with magnetic resonance imaging.

73. The method of claim 69 wherein:
step (b) comprises allowing the compound to bind to GSK-3 at the tissue or cell site to be imaged.

74. The method of claim 69 wherein:
step (b) comprises allowing the compound to bind to GSK-3α at the tissue or cell site to be imaged.

75. The method of claim 69 wherein:
step (b) comprises allowing the compound to bind to GSK-3β at the tissue or cell site to be imaged.

76. A method for the treatment of a condition involving GSK-3 activity, the method comprising:
administering to a subject having the condition a therapeutically effective amount of the compound of any of claims 1 to 7, or 13 to 24, or 30 to 33, or 39 to 44, or 50 to 55.

77. The method of claim 76 wherein:
the condition is a cancer.

78. The method of claim 76 wherein:
the condition is a liver disease.

79. The method of claim 76 wherein:
the condition is a neurodegenerative disease.

80. The method of claim 76 wherein:
the condition is a psychiatric disease.

81. The method of claim 76 wherein:
the condition is Alzheimer's disease.

82. A method for detecting or ruling out a condition involving GSK-3 activity in a subject, the method comprising:
(a) administering to a subject a detectable amount of the compound of any of claims 1 to 7, or 13 to 24, or 30 to 33, or 39 to 44, or 50 to 55 wherein the compound is targeted to GSK-3 at a tissue or cell site in the subject; and
(b) acquiring an image of the cells or tissues to detect the presence or absence of GSK-3 in the subject.

83. The method of claim 82 wherein:
step (b) comprises acquiring the image using an imaging method selected from positron emission tomography imaging, positron emission tomography with computed tomography imaging, and positron emission tomography with magnetic resonance imaging.

84. The method of claim 82 wherein:
the condition is a cancer.

85. The method of claim 82 wherein:
the condition is a liver disease.

86. The method of claim 82 wherein:
the condition is a neurodegenerative disease.

87. The method of claim 82 wherein:
the condition is a psychiatric disease.

88. The method of claim 82 wherein:
the condition is Alzheimer's disease.
